(19) United States
(12) Patent Application Publication
(10) Pub. No.: US 2024/0393334 A1
(43) Pub. Date: Nov. 28, 2024

(54) SYSTEMS AND METHODS FOR DETERMINING UCH-L1, GFAP, AND OTHER BIOMARKERS IN BLOOD SAMPLES

(71) Applicant: Abbott Laboratories, Abbott Park, IL (US)

(72) Inventors: Tony K. Lee, Abbott Park, IL (US); Peter J. Karabatsos, Abbott Park, IL (US); Andrew S. Schapals, Abbott Park, IL (US); Antti Virtanen, Princeton, NJ (US); Beth McQuiston, Abbott Park, IL (US); Craig A. Jeffrey, Princeton, NJ (US); Alison Taylor, Abbott Park, IL (US); Raj Chandran, Abbott Park, IL (US); Jaime Marino, Abbott Park, IL (US); Saul Datwyler, Abbott Park, IL (US)

(21) Appl. No.: 18/743,218

(22) Filed: Jun. 14, 2024

Related U.S. Application Data
(63) Continuation of application No. PCT/US2022/081763, filed on Dec. 16, 2022.
(60) Provisional application No. 63/423,118, filed on Nov. 7, 2022.
(60) Provisional application No. 63/402,132, filed on Aug. 30, 2022.
(60) Provisional application No. 63/402,115, filed on Aug. 30, 2022.
(60) Provisional application No. 63/333,841, filed on Apr. 22, 2022.
(60) Provisional application No. 63/309,033, filed on Feb. 11, 2022.
(60) Provisional application No. 63/309,031, filed on Feb. 11, 2022.
(60) Provisional application No. 63/308,287, filed on Feb. 9, 2022.
(60) Provisional application No. 63/333,836, filed on Apr. 22, 2022.
(60) Provisional application No. 63/291,287, filed on Dec. 17, 2021.

(51) Int. Cl. G01N 33/573; A61B 5/15; G01N 33/68; G01N 33/76; G01N 33/78

(52) U.S. Cl. CPC G01N 33/573; A61B 5/150022; G01N 33/6896; G01N 33/76; G01N 33/78; G01N 2333/59; G01N 2333/9123; G01N 2333/916; G01N 2800/2871

(57) ABSTRACT

Disclosed herein are systems and methods for determining ubiquitin carboxy-terminal hydrolase L1 (UCH-L1), glial fibrillary acidic protein (GFAP), or a combination thereof in a blood sample obtained from a subject. Also disclosed herein are systems and methods for determining CK-MB, β-hCG, thyroid stimulating hormone (TSH), homocysteine, free thyroxine (free T4) or any combinations thereof in a blood sample.

### RELATED APPLICATION INFORMATION

This application is a continuation of International Application No. PCT/US2022/081763 filed on Dec. 16, 2022, which claims priority to U.S. Application No. 63/291,287 filed on Dec. 17, 2021, U.S. Application No. 63/308,287 filed on Feb. 9, 2022, U.S. Application No. 63/309,031 filed on Feb. 11, 2022, U.S. Application No. 63/309,033 filed on Feb. 11, 2022, U.S. Application No. 63/333,836, filed on Apr. 22, 2022, U.S. Application No. 63/333,841, filed on Apr. 22, 2002, U.S. Application No. 63/402,115, filed on Aug. 30, 2022, U.S. Application No. 63/402,132 filed on Aug. 30, 2022, and U.S. Application No. 63/423,118, filed Nov. 7, 2022, the contents of each of which are herein incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 7,900 Byte XML file named “40687-601-SQL_ST26.XML,” created on Dec. 16, 2022.

### TECHNICAL FIELD

The present disclosure relates to systems and methods for determining the amount of ubiquitin carboxy-terminal hydrolase L1 (UCH-L1), glial fibrillary acidic protein (GFAP), or a combination thereof in a blood sample (e.g., capillary blood sample and/or venous blood sample) obtained from a subject. The amount of UCH-L1, GFAP, or a combination thereof in a blood sample obtained from a subject can aid in the diagnosis and evaluation of whether the subject has sustained, may have sustained, or is suspected of sustaining an injury to the head, such as a traumatic brain injury (TBI). The present disclosure further relates to systems and methods for determining the amount of CK-MB, β-hCG, thyroid stimulating hormone (TSH), homocysteine, free thyroxine (free T4) or any combinations thereof in a blood sample (e.g., capillary blood sample and/or venous blood sample) obtained from a subject.

### BACKGROUND

Biological samples for use in laboratory testing to determine the amount or presence of one or more analytes of interest are typically obtained by way of venipuncture by a trained phlebotomist or nurse and typically involve inserting a needle or syringe into a vein on a subject. Unfortunately, venipuncture may be difficult or impractical in certain circumstances such as in newborn infants, the elderly, subjects afraid of needles and/or syringes, and/or in locations not near a hospital or medical clinic. Once a venous blood sample is collected from a subject, the sample is typically packaged and transferred to a processing center for analysis.

Unfortunately, conventional sample collection and testing of venous blood samples has drawbacks. For example, except for the most basic of tests, most blood tests require a substantially higher volume of venous blood from the subject. Because of the high volume of sample required, extraction of blood from alternate sample sites on a subject, which may be less painful and/or less invasive, are often disfavored as they do not yield the volume of sample needed for conventional testing methodologies. Thus, there is a need in the art for methods of determining the amount and/or presence of one or more analytes of interest in a biological sample obtained from a subject that do not require obtaining a venous blood sample from the subject.

### SUMMARY

In one aspect, the present disclosure relates to a method. In one aspect, the present disclosure relates to a method. The method comprises the steps of:
(A) performing at least one assay for (i) ubiquitin carboxy-terminal hydrolase L1 (UCH-L1), glial fibrillary acidic protein (GFAP), or a combination thereof; or (ii) CK-MB, β-hCG, thyroid stimulating hormone (TSH), homocysteine, free thyroxine (free T4), or any combination thereof, on a capillary blood sample obtained from a subject to determine an amount of (i) UCH-L1, GFAP, or a combination thereof; or (ii) CK-MB, β-hCG, TSH, homocysteine, free T4, or any combination thereof; and
(B) communicating the amount of (i) UCH-L1, GFAP, or combination thereof; or (ii) CK-MB, β-hCG, TSH, homocysteine, free T4, or any combination thereof, determined in the sample,
using a point-of-care device or a non-point-of-care device,
wherein the sample is collected from a location on the subject other than a digit and (1) in a decentralized setting; (2) without the use of a syringe, standard needle, or combination thereof; (3) by a user not trained in collecting blood samples from a subject; (4) by a robot; (5) by a self-or other-administered blood collection device; or (6) any combination of (1)-(5), and
further wherein: (i) the assay is capable of being performed in less than about 30 minutes; (ii) the amount of (a) UCH-L1, GFAP, or combination thereof; or (B) CK-MB, β-hCG, TSH, homocysteine, free T4, or any combination thereof, determined in the sample is capable of being communicated in less than about 30 minutes from the time the sample is collected; or (iii) a combination of (i) and (ii).

In some aspects of the above method, the sample is collected using a microsampling device.

In other aspects of the above method, the sample is processed prior to performing the assay and further wherein the sample is whole blood and is processed into serum or plasma.

In some aspects, the microsampling device: (a) comprises a plasma separation device; or (b) is operably linked to the plasma separation device.

In still further aspects of the above method, the assay comprises contacting the sample with:
(I) (a) an anti-UCH-L1 antibody that binds to UCH-L1 to determine the amount of UCH-L1 in the sample; (b) an anti-GFAP antibody that binds to GFAP to determine the amount of GFAP in the sample; or (c) a combination of (a) and (b); or
(II) (a) an anti-CK-MB antibody that binds to CK-MB to determine the amount of CK-MB in the sample; (b) an anti-β-hCG antibody that binds to β-hCG to determine the amount of β-hCG in the sample; (c) an anti-TSH antibody that binds to TSH to determine the amount of TSH in the sample; (d) an anti-homocysteine antibody that binds to homocysteine to determine the amount of homocysteine in the sample; (e) an anti-free T4 antibody that binds to free T4 to determine the amount of free T4 in the sample; or (f) any combination of (a) to (e).

In yet further aspects of the above method, when the assay is for:
(I) (a) GFAP, the GFAP assay comprises a conversion factor for GFAP in a capillary sample compared to GFAP in a venous sample of about 1.0:1.0; (b) UCH-L1, the UCH-L1 assay comprises a conversion factor for UCH-L1 in a capillary sample compared to UCH-L1 in a venous sample of about 2.5:1.0 to about 1.5:1.0; or (c) a combination of (a) and (b); or
(II) (a) CK-MB, the CK-MB assay comprises a conversion factor for CK-MB in a capillary sample compared to CK-MB in a venous sample of about 0.5:1.0 to about 1:0:1.2; (b) β-hCG, the β-hCG assay comprises a conversion factor for β-hCG in a capillary sample compared to β-hCG in a venous sample of about 0.8:1.0 to about 1:0:1.4; (c) TSH, the TSH assay comprises a conversion factor for TSH in a capillary sample compared to TSH in a venous sample of about 0.75:1.0 to about 1.2:1.0; (c) homocysteine, the homocysteine assay comprises a conversion factor for homocysteine in a capillary sample compared to homocysteine in a venous sample of about 1.2:1.0 to about 0.9:1.0; (e) free T4, the free T4 assay comprises a conversion factor for free T4 in a capillary sample compared to free T4 in a venous sample of about 0.8:1.0 to about 1.2:1.0; or (f) any combination of (a) to (e).

In yet further aspects of the above method, when the assay is for: (a) GFAP, UCH-L1, or GFAP and UCH-L1, the method is used to aid in a diagnosis and evaluation of a subject that has sustained or may have sustained an injury to the head; (b) CK-MB, the method is used to diagnose myocardial infarction in a subject; (c) β-hCG, the method is used to determine if a subject is pregnant; (d) TSH, the method is used to assess thyroid function in a subject, diagnose thyroid disease in a subject, treat thyroid disease in a subject, or any combinations thereof; (e) homocysteine, the method is used to diagnose hyperhomocysteinemia, homocystinuria, or hyperhomocysteinemia and homocystinuria in a subject, or treat subjects having hyperhomocysteinemia, homocystinuria, or hyperhomocysteinemia and homocystinuria; or (f) free T4, the method is used to assess thyroid function in a subject, diagnose thyroid disease in a subject, treat thyroid disease in a subject, or any combination thereof.

In yet further aspects of the above method, the amount of (a) UCH-L1, GFAP, and UCH-L1 and UCH-L1 and GFAP; or (b) CK-MB, β-hCG, TSH, homocysteine, free T4, or any combination thereof, is communicated in: (a) about 25 minutes from the time the sample is collected; (b) less than about 20 minutes from the time the sample is collected; (c) about 4 to about 20 minutes from the time the sample is collected; (d) about 15 to about 18 minutes from the time the sample is collected; or (e) less than about 18 minutes from the time the sample is collected.

In yet further aspects, the communicating of the amount of (i) UCH-L1, GFAP, or combination thereof; or (ii) CK-MB, β-hCG, TSH, homocysteine, free T4, or any combination thereof, determined in the sample involves communicating the level of: (i) UCH-L1, GFAP, or combination thereof; or (ii) CK-MB, β-hCG, TSH, homocysteine, free T4, or any combination thereof, in the sample.

In still further aspects of the above method, the sample is processed using a plasma separation device.

In still further aspects, of the above method, the plasma separation device is an apparatus comprising:
a hydrophobic layer comprising at least one microchannel having a first and second channel which defines a path for capillary fluid flow; and
a top layer flanking the hydrophobic layer, wherein a surface of the top layer facing the hydrophobic layer is hydrophilic, wherein the sample comprises blood or blood products.

In some aspects, the apparatus further comprises a bottom layer that flanks the hydrophobic layer, wherein a surface of the bottom layer facing the hydrophobic layer is hydrophilic.

In still other aspects, the top layer, hydrophobic layer, bottom layer, or any combination thereof are adherent to each other.

In still yet another aspect, the at least one microchannel extends longitudinally along a portion of the hydrophobic layer to an opening at a second end.

In still yet a further aspect, the at least one microchannel is less than about 80 mm in length.

In still yet another aspect, the at least one microchannel is less than about 5 mm wide.

In still yet another aspect, the top layer, bottom layer or top and bottom layers are entirely hydrophilic.

In still a further aspect, the composition of the entirety of the top layer, bottom layer, or both the top and bottom layers each comprise same or different materials.

In still further aspects, the hydrophobic layer, top layer, bottom layer or any combination thereof have a combined thickness of about 100 to about 600 microns.

In yet another aspect, the hydrophobic layer has thickness of about 100 to about 200 microns.

In still a further aspect, each of the top layer, bottom layer or top and bottom layers have a thickness of about 50 to about 200 microns.

In yet another aspect, the top layer of the apparatus comprises a sample inlet. In yet other aspects, when the top layer comprises a sample inlet, the hydrophobic layer and optionally, the bottom layer can each comprise an opening below the sample inlet. In still further aspects, the sample inlet comprises a separation membrane. In yet further aspects, the separation membrane is a plasma separation membrane. In yet other aspects, the apparatus further comprises a hydrophilic mesh or hydrophilic film positioned above the separation membrane, below the separation membrane, or both above and below the separation membrane. In yet further aspects, the opening in the hydrophobic layer is connected to a first end of the at least one microchannel.

In yet another aspect, the apparatus further comprises an agglutinating agent. In some aspects, the agglutinating agent comprises lectin (e.g., soybean lectin), Merquat-100, Concanavalin A, DEAE-Dextran, poly-L-lysine, polyvinylpyrrolidone, poly(2-(dimethylamino)ethylmethacrylate), or any combinations thereof. In yet further aspects, the agglutinating agent is coated on or incorporated into the separation membrane, the top layer, the hydrophobic layer, the bottom layer, or any combinations thereof.

In yet further aspects, the plasma separation device is an apparatus comprising:
a container or tube having an inlet and outlet end;
a blood holding chamber at the inlet end of the container or tube;
a detachable serum holding chamber at the outlet end of the container or tube;
a material located within the container between the blood holding chamber and the serum holding chamber for separating components of whole blood based on size.

In still yet other aspects of the above method, the material used in the apparatus employed in the plasma separation comprises glass or porous beads, membranes, a filter, glass or other fiber materials, or any combination thereof.

In still further aspects, the filter described above permits the passage of particles or molecules smaller than about 0.7 microns, about 0.6 microns, about 0.5 microns, about 0.4 microns, or about 0.3 microns.

In still further aspects, in the apparatus used in the plasma separation, a pressure differential between the blood holding chamber and the serum holding chamber allows for whole blood to travel from the blood holding chamber through the filter to produce serum and/or plasma which is collected in the serum holding chamber.

In yet further aspects of the above method, the plasma separation device is operably linked to the microsampling device.

In still further aspects of the above method, the microsampling device comprises a plasma separation device.

In yet further aspects of the above method, the plasma separation device comprises a filter, a membrane, synthetic paper, or any combinations thereof.

In still further aspects of the above method, (a) the point-of-care device comprises a cartridge; or (b) the non-point-of-care device is a higher throughput assay analyzer.

In still further aspects of the above method, the amount of the UCH-L1, GFAP, CK-MB, β-hCG, TSH, homocysteine, free T4, or any combination thereof, is communicated by (a) the point-of-care device or non-point-of-care device in a document and/or spreadsheet, on a mobile device, on a computer, on a website, in an e-mail, or any combination thereof; or (b) displaying on the point-of-care device or non-point-of-care device.

In still further aspects of the above method, the assay used in the above method can be an analog assay, a digital assay, or a combination of an analog assay or a digital assay.

In still further aspects of the above method, the subject is a human.

In another aspect, the present disclosure relates to a system. The system can comprise:
a microsampling device to collect a capillary blood sample from a subject;
a reaction vessel that receives the capillary blood sample and comprises an assay for (i) ubiquitin carboxy-terminal hydrolase L1 (UCH-L1), glial fibrillary acidic protein (GFAP) or a combination thereof; or (ii) CK-MB, β-hCG, TSH, homocysteine, free T4, or any combination thereof; and
an instrument to analyze the reaction vessel to provide an amount of (i) ubiquitin carboxy-terminal hydrolase L1 (UCH-L1), glial fibrillary acidic protein (GFAP) or a combination thereof; or (ii) CK-MB, β-hCG, TSH, homocysteine, free T4, or any combination thereof, in the sample.

In yet another aspect, the system further comprises a plasma separation device to create a processed capillary blood sample.

In some aspects, the plasma separation device comprises an apparatus having:
a) a hydrophobic layer comprising at least one microchannel having a first and second end and which defines a path for capillary fluid flow; and a top layer flanking the hydrophobic layer, wherein a surface of the top layer facing the hydrophobic layer is hydrophilic; or
b) a pre-evacuated container or tube having an inlet and outlet end; a blood holding chamber at the inlet end of the container or tube; a detachable serum holding chamber at the outlet end of the container or tube; a filter located within the container between the blood holding chamber and the serum holding chamber.

In still further aspects, the apparatus used in the plasma separation in a) further comprises a bottom layer flanking the hydrophobic layer, wherein a surface of the bottom layer facing the hydrophobic layer is hydrophilic.

In further aspects, the top layer, hydrophobic layer, bottom layer, or any combination thereof are adherent to each other.

In yet another aspect in the above system, the at least one microchannel extends longitudinally along a portion of the hydrophobic layer to an opening at a second end.

In still another aspect of the above system, the at least one microchannel is less than about 80 mm in length.

In still yet another aspect of the above system, the at least one microchannel is less than about 5 mm wide.

In still yet a further aspect of the above system, the top layer, bottom layer or top and bottom layers are entirely hydrophilic.

In still yet another aspect of the above system, a composition of the entirety of the top layer, bottom layer, or both the top and bottom layers each comprise same or different materials.

In still yet a further aspect of the above system, the hydrophobic layer, top layer, bottom layer or any combination thereof have a combined thickness of about 100 to about 600 microns.

In still yet a further aspect of the above system, the hydrophobic layer has thickness of about 50 to about 200 microns.

In still yet a further aspect of the above system, each of the top layer, bottom layer, or top and bottom layers have a thickness of about 50 to about 200 microns.

In still yet a further aspect of the above system, the top layer comprises a sample inlet.

In still yet a further aspect of the above system, the hydrophobic layer and optionally, the bottom layer, comprise an opening below the sample inlet.

In still yet a further aspect of the above system, the sample inlet comprises a separation membrane.

In still yet a further aspect of the above system, the separation membrane is a plasma separation membrane.

In still yet a further aspect of the above system, the system further comprises a hydrophilic mesh or hydrophilic film positioned above the separation membrane, below the separation membrane, or both above and below the separation membrane.

In still a further aspect of the above system, the opening in the hydrophobic layer is connected to the first end of the at least one microchannel.

In still a further aspect of the above system, wherein the system further comprises an agglutinating agent. In some aspects, the agglutinating agent is coated or incorporated into or on one or more of the top layer, hydrophobic layer, bottom layer, upper substrate, separation membrane, hydrophilic mesh or hydrophilic film, or any combination thereof. In some aspects, the agglutinating agent comprises lectin, Merquat-100, Concanavalin A, DEAE-Dextran, poly-L-lysine, polyvinylpyrrolidone, poly(2-(dimethylamino)ethylmethacrylate), or any combinations thereof.

In yet other aspects of the above system, the filter permits the passage of particles or molecules smaller than about 0.7 microns, about 0.6 microns, about 0.5 microns, about 0.4 microns, or about 0.3 microns.

In still further aspects of the above system, a pressure differential between the blood holding chamber and the serum holding chamber allows for whole blood to travel from the blood holding chamber through the filter to produce serum and/or plasma which is collected in the serum holding chamber.

In still yet another aspect of the system, the reaction vessel comprises an aperture.

In still yet another aspect of the system, the microsampling device includes a housing, a microneedle, a lancet, a microlancet, a blade, a microblade, a microscrew, or any combination thereof coupled to the housing, and a receptacle coupled to the housing; wherein the capillary blood sample is collected in the receptacle.

In still yet another aspect of the system, the receptacle is removably coupled to the housing.

In still yet another aspect of the system, the microsampling device further comprises a cap coupled to the receptacle, wherein the cap seals the capillary blood sample within the receptacle.

In still yet another aspect of the system, the microsampling device further comprises an actuator movable relative to the housing.

In still yet other aspects, in the system, the plasma separation device is in fluid communication with the aperture at any point along the reaction vessel. In yet further aspects, the plasma separation device is placed in fluid communication with the aperture at one end, on a side, or in the middle of the reaction vessel. In yet still further aspects, the plasma separation device is placed in fluid communication with the aperture at an end or side of the reaction vessel at an angle.

In still yet another aspect, the system further comprises a transfer tube. In some additional aspects, the transfer tube comprises a cap or a stopper.

In still yet another aspect of the system, the plasma separation device includes an inlet to receive the capillary blood sample from the microsampling device and an outlet through which the processed capillary blood sample leaves the plasma separation device. In some aspects, the outlet of the plasma separation device is in fluid communication with the aperture of the reaction vessel. In still yet another aspect, the outlet of the plasma separation device is in fluid communication with the cap or stopper of the transfer tube.

In still yet a further aspect of the system, the cap or stopper of the transfer tube is in fluid communication with the aperture of the reaction vessel.

In yet still another aspect of the system, the receptacle is squeezed to force the capillary blood sample through the plasma separation device and into the reaction vessel or transfer tube.

In yet another aspect of the system, the receptacle includes a plunger to force the capillary blood sample through the plasma separation device and into the reaction vessel or transfer tube.

In still further aspects of the system, the plasma separation device is integrated within the receptacle.

In yet other aspects of the system, the receptacle is a reaction vessel.

In further aspects of the system, the plasma separation device is integrated within the reaction vessel.

In yet other aspects in the system, the plasma separation device is integrated into the transfer tube.

In still a further aspect of the system, the plasma separation device includes a filter, a membrane, a synthetic paper, or any combinations thereof.

In still yet a further aspect of the system, the amount of (i) UCH-L1, GFAP, or UCH-L1 and GFAP; or (ii) CK-MB, β-hCG, TSH, homocysteine, free T4, or any combination thereof, is determined in (a) about 25 minutes from the time the sample is collected; (b) less than about 20 minutes from the time the sample is collected; (c) about 4 to about 20 minutes from the time the sample is collected; (d) about 15 to about 18 minutes from the time the sample is collected; or (e) less than about 18 minutes from the time the sample is collected.

In yet a further aspect of the system, the amount of (i) UCH-L1, GFAP, or UCH-L1 and GFAP; or (ii) CK-MB, β-hCG, TSH, homocysteine, free T4, or any combination thereof, is communicated by the instrument. In further aspects, the amount of (i) UCH-L1, GFAP, or UCH-L1 and GFAP; or (ii) CK-MB, β-hCG, TSH, homocysteine, free T4, or any combination thereof, is communicated in a document and/or spreadsheet, on a mobile device, on a computer, on a website, in an e-mail, or any combination thereof. In still yet another aspect, the amount of (i) UCH-L1, GFAP, or UCH-L1 and GFAP; or (ii) CK-MB, β-hCG, TSH, homocysteine, free T4, or any combination thereof, is communicated by displaying on the instrument (e.g., a point-of-care instrument, a non-point of care instrument, etc.). In still yet another aspect, at least a portion of the system is usable in a decentralized setting.

In still further aspects of the above system, the communicating of the amount of (i) UCH-L1, GFAP, or a combination thereof; or (ii) CK-MB, β-hCG, TSH, homocysteine, free T4, or any combination thereof, determined in the sample involves communicating the level of (i) UCH-L1, GFAP, or a combination thereof; or (ii) CK-MB, β-hCG, TSH, homocysteine, free T4, or any combination thereof, in the sample.

In yet another aspect, the present disclosure relates to a method comprising:
performing at least one assay for (i) ubiquitin carboxy-terminal hydrolase L1 (UCH-L1), glial fibrillary acidic protein (GFAP), or a combination thereof; or (2) CK-MB, β-hCG, TSH, homocysteine, free T4, or any combination thereof, on a blood sample obtained from a subject to determine an amount of (i) UCH-L1, GFAP, or UCH-L1 and GFAP; or (ii) CK-MB, β-hCG, TSH, homocysteine, free T4, or any combination thereof; and
communicating the amount of (i) UCH-L1, GFAP, or UCH-L1 and GFAP; or (ii) CK-MB, β-hCG, TSH, homocysteine, free T4, or any combination thereof, determined in the sample,
wherein the sample is collected with the use of a syringe, standard needle, or combination thereof; and
further wherein the sample is processed prior to performing the assay with a plasma separation device comprising an apparatus having:
a) a hydrophobic layer comprising at least one microchannel having a first and second end and which defines a path for capillary fluid flow; and a top layer flanking the hydrophobic layer, wherein a surface of the top layer facing the hydrophobic layer is hydrophilic; or
b) a pre-evacuated container or tube having an inlet and outlet end;
a blood holding chamber at the inlet end of the container or tube;
a detachable serum holding chamber at the outlet end of the container or tube;
a filter located within the container between the blood holding chamber and the serum holding chamber.

In the above method the blood sample is a venous blood sample or a capillary blood sample. In some aspects, the sample is collected in a decentralized or a centralized setting.

In still further aspects of the above method, the assay comprises contacting the sample with:
(I) (a) an anti-UCH-L1 antibody that binds to UCH-L1 to determine the amount of UCH-L1 in the sample; (b) an anti-GFAP antibody that binds to GFAP to determine the amount of GFAP in the sample; or (c) a combination of (a) and (b); or
(II) (a) an anti-CK-MB antibody that binds to CK-MB to determine the amount of CK-MB in the sample; (b) an anti-β-hCG antibody that binds to β-hCG to determine the amount of β-hCG in the sample; (c) an anti-TSH antibody that binds to TSH to determine the amount of TSH in the sample; (d) an anti-homocysteine antibody that binds to homocysteine to determine the amount of homocysteine in the sample; (e) an anti-free T4 antibody that binds to free T4 to determine the amount of free T4 in the sample; or (f) any combination of (a) to (e).

In yet further aspects of the above method, when the blood sample is capillary blood, and when the assay is for:
(I) (a) GFAP, the GFAP assay comprises a conversion factor for GFAP in a capillary sample compared to GFAP in a venous sample of about 1.0:1.0; (b) UCH-L1, the UCH-L1 assay comprises a conversion factor for UCH-L1 in a capillary sample compared to UCH-L1 in a venous sample of about 2.5:1.0 to about 1.5:1.0; or (c) a combination of (a) and (b); or
(II) (a) CK-MB, the CK-MB assay comprises a conversion factor for CK-MB in a capillary sample compared to CK-MB in a venous sample of about 0.5:1.0 to about 1:0:1.2; (b) β-hCG, the β-hCG assay comprises a conversion factor for β-hCG in a capillary sample compared to β-hCG in a venous sample of about 0.8:1.0 to about 1.0:1.4; (c) TSH, the TSH assay comprises a conversion factor for TSH in a capillary sample compared to TSH in a venous sample of about 0.75:1.0 to about 1.2:1.0; (d) homocysteine, the homocysteine assay comprises a conversion factor for homocysteine in a capillary sample compared to homocysteine in a venous sample of about 1.2:1.0 to about 0.9:1.0; (e) free T4, the free T4 assay comprises a conversion factor for free T4 in a capillary sample compared to free T4 in a venous sample of about 0.8:1.0 to about 1.2:1.0; or (f) any combination of (a) to (e).

In yet further aspects of the above method, when the assay is for: (a) GFAP, UCH-L1, or GFAP and UCH-L1, the method is used to aid in a diagnosis and evaluation of a subject that has sustained or may have sustained an injury to the head; (b) CK-MB, the method is used to diagnose myocardial infarction in a subject; (c) β-hCG, the method is used to determine if a subject is pregnant; (d) TSH, the method is used to assess thyroid function in a subject, diagnose thyroid disease in a subject, treat thyroid disease in a subject, or any combinations thereof; (e) homocysteine, the method is used to diagnose hyperhomocysteinemia, homocystinuria, or hyperhomocysteinemia and homocystinuria in a subject, or treat subjects having hyperhomocysteinemia, homocystinuria, or hyperhomocysteinemia and homocystinuria; or (f) free T4, the method is used to assess thyroid function in a subject, diagnose thyroid disease in a subject, treat thyroid disease in a subject, or any combination thereof.

In yet further aspects of the above method, the amount of (a) UCH-L1, GFAP, and UCH-L1 and UCH-L1 and GFAP; or (b) CK-MB, β-hCG, TSH, homocysteine, free T4, or any combination thereof, is communicated in: (a) about 25 minutes from the time the sample is collected; (b) less than about 20 minutes from the time the sample is collected; (c) about 4 to about 20 minutes from the time the sample is collected; (d) about 15 to about 18 minutes from the time the sample is collected; or (e) less than about 18 minutes from the time the sample is collected.

In still further aspects of the above method, the amount of the UCH-L1, GFAP, CK-MB, β-hCG, TSH, homocysteine, free T4, or any combination thereof, is communicated by (a) a point-of-care device or non-point-of-care device in a document and/or spreadsheet, on a mobile device, on a computer, on a website, in an e-mail, or any combination thereof; or (b) displaying on a point-of-care device or non-point-of-care device.

In still further aspects of the above method, the communicating of the amount of (i) UCH-L1, GFAP, or a combination thereof; or (ii) CK-MB, β-hCG, TSH, homocysteine, free T4, or any combination thereof, determined in the sample involves communicating the level of (i) UCH-L1, GFAP, or a combination thereof; or (ii) CK-MB, β-hCG, TSH, homocysteine, free T4, or any combination thereof, in the sample.

In still further aspects of the above method, the assay used in the above method can be an analog assay, a digital assay, or a combination of an analog assay or a digital assay. In still further aspects of the above method, the subject is a human.

In yet further aspects of the above the method, the apparatus in a) further comprises a bottom layer flanking the hydrophobic layer, wherein a surface of the bottom layer facing the hydrophobic layer is hydrophilic.

In yet further aspects of the above method, the top layer, hydrophobic layer, bottom layer, or any combination thereof are adherent to each other.

In yet further aspects of the above method, the at least one microchannel extends longitudinally along a portion of the hydrophobic layer to an opening at a second end.

In still yet other aspects of the above method, the at least one microchannel is less than about 80 mm in length.

In still yet further aspects of the above method, the at least one microchannel is less than about 5 mm wide.

In still yet further aspects of the above method, the top layer, bottom layer or top and bottom layers are entirely hydrophilic.

In still yet further aspects of the above method, the composition of the entirety of the top layer, bottom layer, or both the top and bottom layers each comprise same or different materials.

In still yet further aspects of the above method, the hydrophobic layer, top layer, bottom layer or any combination thereof have a combined thickness of about 100 to about 600 microns.

In still yet further aspects of the above method, the hydrophobic layer has thickness of about 50 to about 200 microns.

In still yet further aspects of the above method, each of the top layer, bottom layer, or top and bottom layers have a thickness of about 50 to about 200 microns.

In still yet further aspects of the above method, the top layer comprises a sample inlet.

In still yet further aspects of the above method, the hydrophobic layer and optionally, the bottom layer, comprise an opening below the sample inlet.

In still yet further aspects of the above method, the sample inlet comprises a separation membrane.

In still yet further aspects of the above method, the separation membrane is a plasma separation membrane.

In still yet further aspects of the above method, the separation membrane (e.g., plasma separation membrane) further comprises a hydrophilic mesh or hydrophilic film positioned above the separation membrane, below the separation membrane, or both above and below the separation membrane.

In yet further aspects of the above method, the opening in the hydrophobic layer is connected to the first end of the at least one microchannel.

In yet further aspects of the above method, the method further comprises an agglutinating agent. In some aspects, the agglutinating agent is coated or incorporated into or on one or more of the top layer, hydrophobic layer, bottom layer, upper substrate, separation membrane, hydrophilic mesh or hydrophilic film, or any combination thereof.

In yet further aspects of the above method, the agglutinating agent comprises lectin, Merquat-100, Concanavalin A, DEAE-Dextran, poly-L-lysine, polyvinylpyrrolidone, poly(2-(dimethylamino)ethylmethacrylate), or any combinations thereof.

In further aspects of the above method, the filter permits the passage of particles or molecules smaller than about 0.7 microns, about 0.6 microns, about 0.5 microns, about 0.4 microns, or about 0.3 microns.

In still yet further aspects of the above method, a pressure differential between the blood holding chamber and the serum holding chamber allows for whole blood to travel from the blood holding chamber through the filter to produce serum and/or plasma which is collected in the serum holding chamber.

In yet another aspect, the present disclosure relates to a system comprising:
a plasma separation device to process a whole blood sample obtained from a subject into serum and/or plasma;
a reaction vessel that receives the serum and/or plasma from the subject and comprises an assay for (i) ubiquitin carboxy-terminal hydrolase L1 (UCH-L1), glial fibrillary acidic protein (GFAP), or a combination thereof; or (ii) CK-MB, β-hCG, TSH, homocysteine, free T4, or any combination thereof; and
an instrument to analyze the reaction vessel to provide an amount of (i) UCH-L1, GFAP, or UCH-L1 and GFAP; or (ii) CK-MB, β-hCG, TSH, homocysteine, free T4, or any combination thereof, in the sample,
wherein the plasma separation device comprises an apparatus having:
a) a hydrophobic layer comprising at least one microchannel having a first and second end and which defines a path for capillary fluid flow; and a top layer flanking the hydrophobic layer, wherein a surface of the top layer facing the hydrophobic layer is hydrophilic; or
b) a pre-evacuated container or tube having an inlet and outlet end;
a blood holding chamber at the inlet end of the container or tube;
a detachable serum holding chamber at the outlet end of the container or tube;
a filter located within the container between the blood holding chamber and the serum holding chamber.

In still yet a further aspect of the system, the amount of (i) UCH-L1, GFAP, or UCH-L1 and GFAP; or (ii) CK-MB, β-hCG, TSH, homocysteine, free T4, or any combination thereof, is determined in (a) about 25 minutes from the time the sample is collected; (b) less than about 20 minutes from the time the sample is collected; (c) about 4 to about 20 minutes from the time the sample is collected; (d) about 15 to about 18 minutes from the time the sample is collected; or (e) less than about 18 minutes from the time the sample is collected.

In some aspects, in the above system, the blood sample is a venous blood sample or a capillary blood sample.

In yet other aspects, in the above system, the sample is collected in a decentralized or a centralized setting.

In yet further aspects, in the above system, where the apparatus in a) further comprises a bottom layer flanking the hydrophobic layer, wherein a surface of the bottom layer facing the hydrophobic layer is hydrophilic.

In still yet other aspects, in the above system, the top layer, hydrophobic layer, bottom layer, or any combination thereof are adherent to each other.

In yet further aspects, in the above system, the at least one microchannel extends longitudinally along a portion of the hydrophobic layer to an opening at a second end.

In yet further aspects, in the above system, the at least one microchannel is less than about 80 mm in length.

In yet further aspects, in the above system, the at least one microchannel is less than about 5 mm wide.

In yet further aspects, in the above system, the top layer, bottom layer or top and bottom layers are entirely hydrophilic.

In yet further aspects, in the above system, a composition of the entirety of the top layer, bottom layer, or both the top and bottom layers each comprise same or different materials.

In yet further aspects, in the above system, the hydrophobic layer, top layer, bottom layer or any combination thereof have a combined thickness of about 100 to about 600 microns.

In yet further aspects, in the above system, the hydrophobic layer has thickness of about 50 to about 200 microns.

In yet further aspects, in the above system, each of the top layer, bottom layer, or top and bottom layers have a thickness of about 50 to about 200 microns.

In yet further aspects, in the above system, the top layer comprises a sample inlet.

In yet further aspects, in the above system, the hydrophobic layer and optionally, the bottom layer, comprise an opening below the sample inlet.

In yet further aspects, in the above system, the sample inlet comprises a separation membrane.

In yet further aspects, in the above system, the separation membrane is a plasma separation membrane. In yet further aspects, the separation membrane (e.g., plasma separation membrane), further comprises a hydrophilic mesh or hydrophilic film positioned above the separation membrane, below the separation membrane, or both above and below the separation membrane.

In yet further aspects, in the above system, the opening in the hydrophobic layer is connected to the first end of the at least one microchannel.

In yet further aspects of the above system, the system further comprises an agglutinating agent. In some aspects, the agglutinating agent is coated or incorporated into or on one or more of the top layer, hydrophobic layer, bottom layer, upper substrate, separation membrane, hydrophilic mesh or hydrophilic film, or any combination thereof.

In yet further aspects of the above system, the agglutinating agent comprises lectin, Merquat-100, Concanavalin A, DEAE-Dextran, poly-L-lysine, polyvinylpyrrolidone, poly(2-(dimethylamino)ethylmethacrylate), or any combinations thereof.

In yet further aspects of the above system, the filter permits the passage of particles or molecules smaller than about 0.7 microns, about 0.6 microns, about 0.5 microns, about 0.4 microns, or about 0.3 microns.

In yet further aspects of the above system, a pressure differential between the blood holding chamber and the serum holding chamber allows for whole blood to travel from the blood holding chamber through the filter to produce serum and/or plasma which is collected in the serum holding chamber.

In yet a further aspect of the system, the amount of (i) UCH-L1, GFAP, or UCH-L1 and GFAP; or (ii) CK-MB, β-hCG, TSH, homocysteine, free T4, or any combination thereof, is communicated by the instrument. In further aspects, the amount of (i) UCH-L1, GFAP, or UCH-L1 and GFAP; or (ii) CK-MB, β-hCG, TSH, homocysteine, free T4, or any combination thereof, is communicated in a document and/or spreadsheet, on a mobile device, on a computer, on a website, in an e-mail, or any combination thereof. In still yet another aspect, the amount of (i) UCH-L1, GFAP, or UCH-L1 and GFAP; or (ii) CK-MB, β-hCG, TSH, homocysteine, free T4, or any combination thereof, is communicated by displaying on the instrument (e.g., a point-of-care instrument, a non-point of care instrument, etc.). In still yet another aspect, at least a portion of the system is usable in a decentralized setting.

In still further aspects of the above system, the communicating of the amount of (i) UCH-L1, GFAP, or a combination thereof; or (ii) CK-MB, β-hCG, TSH, homocysteine, free T4, or any combination thereof, determined in the sample involves communicating the level of (i) UCH-L1, GFAP, or a combination thereof; or (ii) CK-MB, β-hCG, TSH, homocysteine, free T4, or any combination thereof, in the sample.

### BRIEF DESCRIPTION OF THE DRAWINGS

FIG. 1 shows a system for diagnosis and evaluation of a subject that has sustained or may have sustained an injury to the head according to one aspect of the disclosure.

FIG. 2A-2E shows operation steps of the system of FIG. 1.

FIG. 3 shows additional and alternative aspects of the system of FIG. 1.

FIG. 4 shows additional and alternative aspects of the system of FIG. 1.

FIG. 5 shows additional and alternative aspects of the system of FIG. 1.

FIG. 6A-6C shows additional and alternative aspects of the system of FIG. 1.

FIG. 7 shows one embodiment of an apparatus of the present disclosure that can be used as a plasma separation device.

FIG. 8 shows another embodiment of an apparatus of the present disclosure that can be used as a plasma separation device.

FIG. 9 shows a further embodiment of an apparatus of the present disclosure used as a plasma separation device as described in Example 1.

FIG. 10 shows a device comprising an apparatus of the present disclosure that is operatively linked, removably coupled, or in fluid communication with a sample analysis cartridge. The second end of the microchannel of the apparatus is in fluid communication with a sample application area on the sample analysis cartridge.

FIG. 11 shows the GFAP (pg/mL) concentration readings on the TBI Plasma Cartridge for donors taken in the study described in Example 3. Individual results (left) and mean results with 95% CI's (right) are included. Each error bar was generated using a 95% confidence interval of the mean.

FIG. 12 shows the UCH-L1 concentration readings (pg/mL) on the TBI Plasma Cartridge for donors taken in the study described in Example 3. Individual results (left) and mean results (right) are included.

FIG. 13 shows the GFAP plasma readings (pg/mL) for only venous and capillary plasma samples on the TBI Plasma Cartridge for donors taken in the study described in Example 4. Individual results (left) and mean results with 95% CI's (right) are included. Each error bar was generated using a 95% confidence interval of the mean.

FIG. 14 shows UCH-L1 plasma readings (pg/mL) for only venous and capillary plasma samples on the TBI Plasma Cartridge for donors taken in the study described in Example 4. Individual results (left) and mean results with 95% CI's (right) are included. Each error bar was generated using a 95% confidence interval of the mean.

FIG. 15 shows the CK-MB concentration results individually (left) and by mean with 95% confidence intervals (right) as described in Example 5. Each error bar was generated using a 95% confidence interval of the mean.

FIG. 16 shows the β-hCG concentration results from phase 3.2-3.3 individually (left) and by mean with 95% confidence intervals (right) as described in Example 6. Each error bar was generated using a 95% confidence interval of the mean.

### DETAILED DESCRIPTION

The present disclosure relates to systems and methods for determining the amount of ubiquitin carboxy-terminal hydrolase L1 (UCH-L1), glial fibrillary acidic protein (GFAP), CK-MB, β-hCG, thyroid stimulating hormone (TSH), homocysteine, free thyroxine (free T4) or any combinations thereof in a capillary blood sample obtained from a subject (e.g., such as a human subject). In some aspects, the methods and systems involve performing at least one assay for UCH-L1, GFAP, or a combination thereof. In yet other aspects, the assay involves contacting the capillary blood sample with (a) an anti-UCH-L1 antibody that binds to UCH-L1 to determine the amount of UCH-L1 in the sample; (b) an anti-GFAP antibody that binds to GFAP to determine the amount of GFAP in the sample; or (c) a combination of (a) and (b). In other aspects, the methods and systems involve performing at least one assay for CK-MB, β-hCG, TSH, homocysteine, free thyroxine (free T4) or any combinations thereof. In yet other aspects, the assay involves contacting the capillary blood sample with (a) an anti-CK-MB antibody that binds to CK-MB to determine the amount of CK-B in the sample; (b) an anti-β-hCG antibody that binds to β-hCG to determine the amount of β-hCG in the sample; (c) an anti-TSH antibody that binds to TSH to determine the amount of TSH in the sample; (d) an anti-homocysteine antibody that binds to homocysteine to determine the amount of homocysteine in the sample; (e) an anti-free T4 antibody that binds to free T4 to determine the amount of free T4 in the sample; or (f) any combination of (a) to (e). In still yet other aspects, the methods involve collecting the sample (1) in a decentralized setting; (2) without the use of a syringe, standard needle, or combination thereof; (3) by a user not trained in collecting blood samples from a subject; (4) by a robot; (5) by a self-or other-administered blood collection device; or (6) any combination of (1)-(5). In still further aspects, the amount of UCH-L1, GFAP, CK-MB, β-hCG, TSH, homocysteine, free T4, or any combinations thereof determined in the capillary blood sample is communicated within about 4 to about 40 minutes from the time the sample is collected. In some aspects, amount of UCH-L1, GFAP, or a combination thereof determined in the capillary blood sample obtained according to the methods described herein can be used to aid in the diagnosis and/or evaluation of whether the subject has sustained, may have sustained, or is suspected of sustaining an injury to the head, such as a traumatic brain injury (TBI).

In other aspects, the amount of CK-MB determined in the capillary blood sample obtained according to the methods described herein can be used to diagnose myocardial infarction in a subject. In still other aspects, the amount of β-hCG determined in the capillary blood sample obtained according to the methods described herein can be used to determine if a subject is pregnant. In still other aspects, the amount of TSH determined in the capillary blood sample obtained according to the methods described herein can be used to assess thyroid function in a subject, diagnose thyroid disease in a subject and/or treat thyroid disease in a subject. In still other aspects, the amount of homocysteine determined in the capillary blood sample obtained according to the methods described herein can be used to diagnose hyperhomocysteinemia and/or homocystinuria in a subject or treat subjects having hyperhomocysteinemia and/or homocystinuria. In still other aspects, the amount of free T4 determined in the capillary blood sample obtained according to the methods described herein can be used to assess thyroid function in a subject, diagnose thyroid disease in a subject, treat thyroid disease in a subject, or any combination thereof.

In still yet further other aspects, the methods involve processing the sample prior to performing the assay. In some aspects, the sample is processed using centrifugation. In yet other aspects, the sample is processed using a plasma separation device.

In other aspects, the system includes a microsampling device to collect a capillary blood sample from a subject, and a reaction vessel that receives the capillary blood sample. In additional aspects, the system includes a plasma separation device to create a processed capillary blood sample from the capillary blood. The system further includes an instrument to analyze the reaction vessel to determine the amount of UCH-L1, GFAP, CK-MB, β-hCG, TSH, homocysteine, free T4, or any combinations thereof in the sample.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Affinity matured antibody” is used herein to refer to an antibody with one or more alterations in one or more CDRs, which result in an improvement in the affinity (i.e., K_{D}, k_{d} or kₐ) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies is known in the art, including the screening of a combinatory antibody library that has been prepared using bio-display. For example, Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); and Hawkins et al, J. Mol. Biol., 226:889-896 (1992). Selective mutation at selective mutagenesis positions and at contact or hypermutation positions with an activity-enhancing amino acid residue is described in U.S. Pat. No. 6,914,128 B1.

An “amount” as used herein refers to a quantity specified (e.g., high or low) or a number e.g., where the number is a level, such as a position on a real or imaginary scale of amount or quantity, or a concentration, such as, for example, a relative amount of a given substance contained within a solution or in a particular volume of space, e.g., the amount of solute per unit volume of solution.

An “analog assay” as used herein refers to an assay in which the presence of and/or amount of an analyte in a test sample is determined by measuring the total signal produced (e.g., fluorescence, color, etc.) by the analyte in an entire reaction mixture (e.g., in a reaction vessel). In an analog assay, the noise is indistinguishable from the signal. An example of an analog assay is an assay in which the presence of and/or amount of an analyte is determined by measuring the total signal produced from a plurality of beads or microparticles contained in a reaction vessel.

“Antibody” and “antibodies” as used herein refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25 (11): 1290-1297 (2007) and PCT International Application WO 2001/058956, the contents of each of which are herein incorporated by reference), and functionally active epitope-binding fragments of any of the above. Antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody” (e.g., an anti-UCH-L1 antibody or a UCH-L1 antibody, an anti-GFAP antibody or a GFAP antibody, an anti-CK-MB antibody or a CK-MB antibody, anti-β-hCG antibody or a β-hCG antibody, anti-TSH antibody or a TSH antibody, a anti-homocysteine antibody or homocysteine antibody, or anti-free T4 antibody or free T4 antibody).

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Aperture” as used herein refers to an opening, hole, or gap.

The “area under curve” or “AUC” refers to area under a ROC curve. AUC under a ROC curve is a measure of accuracy. An AUC of 1 represents a perfect test, whereas an AUC of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

“Beta human chorionic gonadotropin (hCG)” or “β-hCG” refers to the beta (B) subunit of human chorionic gonadotropin (hCG). Human chorionic gonadotropin is a sialoglycoprotein with a molecular weight of approximately 46,000 daltons. HCG is initially secreted by the trophoblastic cells of the placenta shortly after implantation of the fertilized ovum into the uterine wall. The rapid rise in hCG serum levels after conception makes it an useful marker for early confirmation and monitoring of pregnancy. Physiologically, hCG appears to maintain the corpus luteum, thereby allowing synthesis of progesterone and estrogens that support the endometrium. As uncomplicated pregnancies progress, the placenta assumes the production of these hormones. The serum hCG levels increase to a peak concentration, then decrease and plateau. HCG circulates as the intact molecule in the serum of normal women who have an uncomplicated pregnancy. The subunits are cleaved rapidly and cleared by the kidney. The placental hormone, hCG, is similar to luteinizing hormone (LH), follicle stimulating hormone (FSH), and thyroid stimulating hormone (TSH). All are glycoproteins consisting of two noncovalently bound dissimilar subunits, designated alpha and beta, with attached carbohydrate sidechains. The alpha subunits of these glycoproteins are very similar. In contrast, the beta subunit portions determine the biological and immunochemical specificities. The beta subunits of hCG and LH exhibit considerable homology in amino acid content. Amino acid residues specific for the beta subunit of hCG confer the immunochemical specificity. In addition, β-hCG also is used to assess other diseases in which this analyte is implicated, e.g., diagnosis and management of tumors, gestational trophoblastic diseases, or hCG-producing germ cell tumors of ovarian, placental, or testicular origin.”

“Bead” and “particle” are used herein interchangeably and refer to a substantially spherical solid support. One example of a bead or particle is a microparticle. Microparticles that can be used herein can be any type known in the art. For example, the bead or particle can be a magnetic bead or magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO·Fe₂O₃). Beads can have a solid core portion that is magnetic and is surrounded by one or more non-magnetic layers. Alternately, the magnetic portion can be a layer around a non-magnetic core. The microparticles can be of any size that would work in the methods described herein, e.g., from about 0.75 to about 5 nm, or from about 1 to about 5 nm, or from about 1 to about 3 nm.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, an IgG molecule, an IgG1 molecule, a monoclonal antibody, a chimeric antibody, a CDR-grafted antibody, a humanized antibody, an affinity matured antibody, and fragments of any such antibodies that retain the ability to bind to an antigen.

“Bispecific antibody” is used herein to refer to a full-length antibody that is generated by quadroma technology (see Milstein et al., Nature, 305 (5934): 537-540 (1983)), by chemical conjugation of two different monoclonal antibodies (see, Staerz et al., Nature, 314 (6012): 628-631 (1985)), or by knob-into-hole or similar approaches, which introduce mutations in the Fc region (see Holliger et al., Proc. Natl. Acad. Sci. USA, 90 (14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences) and is monovalent for each antigen to which it binds to.

“Capillary blood sample” as used herein refers to a sample of blood from the capillaries which is obtained (e.g., extracted) through the skin (and not the veins) of a subject using a syringe, needle, or any other suitable device or combination thereof. For example, a whole blood sample can be obtained from the skin on the fingers and/or toes, a hand, a foot (including the heel), an earlobe, a location on the arms and/or legs, chest, back, head, or any combinations thereof. In some embodiments, the capillary blood sample is whole blood, serum or plasma. In yet other embodiments, the capillary blood sample contains predominantly capillary blood, but may also contain or comprise a small amount or percentage of interstitial fluid.

“Cartridge” as used herein refers to a hollow container and/or chip that comprises one or more substances and/or components (e.g., a liquid, reagents (e.g., antibodies and/or antigens), and/or a particle (e.g., a bead, or microparticle)) for insertion into an apparatus (e.g., a point-of-care device). In some aspects, a cartridge has one or more apertures. In some aspects, a cartridge is a microfluidic cartridge.

“Communicating the amount” as used herein, refers to communicating, as described herein, the amount, in terms of presence (i.e., a qualitative measure), or in terms of a level or levels (i.e., a quantitative measure), such that “amount” refers to presence or level. In some aspects, “communicating the amount” refers to communicating the presence (e.g., qualitative measure) of a biomarker. In other aspects, “communicating the amount” refers to communicating the level (e.g., quantitative measure) of a biomarker.

“Creatine kinase-myoglobin binding” or “CK-MB” refers to an 84,000 molecular weight enzyme that represents a significant fraction of the creatine kinase present in myocardial tissue. The appearance of CK-MB in a biological sample, such as, for example, serum, in the absence of major muscle trauma, may be indicative of cardiac damage and thus, myocardial infarction.

“Coupled” or “linked” as used herein refers to two or more components that are secured, by any suitable means, together. Accordingly, in some embodiments, the statement that two or more parts or components are “coupled” shall mean that the parts are joined or operate together either directly or indirectly, e.g., through one or more intermediate parts or components.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196:901-917 (1987); and Chothia et al., Nature, 342:877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9:133-139 (1995), and MacCallum, J. Mol. Biol., 262 (5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

A “clinically-relevant time frame” refers to a time frame (e.g., seconds, minutes, or hours) during which a careful and prudent medical practitioner (e.g., doctor, nurse, paramedic, or other) would reasonably consider the results of one or more biomarker tests to have bearing on an imaging procedure, such as a head CT scan, or pursuant to guidelines established by an overseeing entity (e.g., a standards-setting body such as the World Health Organization (WHO), physicians review board, regulatory approval authority such as FDA, EMEA or other, etc.).

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient whole blood, serum or plasma sample, in accordance with the methods described herein and other methods known in the art. Some components can be in solution or lyophilized for reconstitution for use in an assay.

“Correlated to” as used herein refers to compared to.

“CT scan” as used herein refers to a computerized tomography (CT) scan. A CT scan combines a series of X-ray images taken from different angles and uses computer processing to create cross-sectional images, or slices, of the bones, blood vessels and soft tissues inside your body. The CT scan may use X-ray CT, positron emission tomography (PET), single-photon emission computed tomography (SPECT), computed axial tomography (CAT scan), or computer aided tomography. The CT scan may be a conventional CT scan or a spiral/helical CT scan. In a conventional CT scan, the scan is taken slice by slice and after each slice the scan stops and moves down to the next slice, e.g., from the top of the abdomen down to the pelvis. The conventional CT scan requires patients to hold their breath to avoid movement artefact. The spiral/helical CT scan is a continuous scan which is taken in a spiral fashion and is a much quicker process where the scanned images are contiguous.

A head CT scan is “negative” for a TBI when no intracranial lesion(s) is observed in an image taken from a subject that has sustained, may have sustained or is suspected of sustaining an injury to the head. To further clarify, the head CT scan of a subject is “negative” for a TBI when a lesion is not found or identified; however, in some aspects, the subject may still be experiencing symptoms (e.g., of TBI) even though the head CT is negative. Most subjects will be negative for a TBI on head CT given that not all injuries or lesions can be visualized by head CT. Consequently, the methods and assays described herein can be used to provide an assessment or determination of a subject with a negative head CT that may still have a TBI.

“Decentralize”, “Decentralized”, or “Decentralization”, as used interchangeably herein, refers to, in the context of testing, the performance of one or more medical tests and/or assays outside of a traditional medical setting (e.g., a hospital, physician office, stand alone lab site, etc.) to one or more places such as urgent care clinics, retail clinics, pharmacies, grocery stores or convenience stores, residences (e.g., homes, apartments, etc.), workplaces, and/or government offices (e.g., U.S. Transportation and Safety Authority), etc. “Hybrid-decentralization” or “hybrid-decentralized” refers to situations in which a subject or patient collects a sample at a residence and/or workplace and ships the sample to a laboratory, avoiding a professional collection site (such as a hospital, physician's office, or stand-alone sample collection or lab site).

“Determined by an assay” is used herein to refer to the determination of a reference level by any appropriate assay. The determination of a reference level may, in some embodiments, be achieved by an assay of the same type as the assay that is to be applied to the sample from the subject (for example, by an immunoassay, clinical chemistry assay, a single molecule detection assay, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS)). The determination of a reference level may, in some embodiments, be achieved by an assay of the same type and under the same assay conditions as the assay that is to be applied to the sample from the subject. As noted herein, this disclosure provides exemplary reference levels (e.g., calculated by comparing reference levels at different time points). It is well within the ordinary skill of one in the art to adapt the disclosure herein for other assays to obtain assay-specific reference levels for those other assays based on the description provided by this disclosure. For example, a set of training samples comprising samples obtained from human subjects known to have sustained an injury to the head (and more particularly, samples obtained from human subjects known to have sustained a (i) mild TBI; and/or (ii) moderate, severe, or moderate to severe TBI and samples obtained from human subjects known not to have sustained an injury to the head may be used to obtain assay-specific reference levels. It will be understood that a reference level “determined by an assay” and having a recited level of “sensitivity” and/or “specificity” is used herein to refer to a reference level which has been determined to provide a method of the recited sensitivity and/or specificity when said reference level is adopted in the methods of the disclosure. It is well within the ordinary skill of one in the art to determine the sensitivity and specificity associated with a given reference level in the methods of the disclosure, for example by repeated statistical analysis of assay data using a plurality of different possible reference levels.

Practically, when discriminating between a subject as having a traumatic brain injury or not having a traumatic brain injury or a subject as having a mild versus a moderate, severe, or moderate to severe traumatic brain injury, the skilled person will balance the effect of raising a cutoff on sensitivity and specificity. Raising or lowering a cutoff will have a well-defined and predictable impact on sensitivity and specificity, and other standard statistical measures. It is well known that raising a cutoff will improve specificity but is likely to worsen sensitivity (proportion of those with disease who test positive). In contrast, lowering a cutoff will improve sensitivity but will worsen specificity (proportion of those without disease who test negative). The ramifications for detecting traumatic brain injury or determining a mild versus moderate, severe, or moderate to severe traumatic brain injury will be readily apparent to those skilled in the art. In discriminating whether a subject has or does not have a traumatic brain injury or a mild versus a moderate, severe, or moderate to severe traumatic brain injury, the higher the cutoff, specificity improves as more true negatives (i.e., subjects not having a traumatic brain injury, not having a mild traumatic brain injury, not have a moderate traumatic brain injury, not having a severe traumatic brain injury or not having a moderate to severe traumatic brain injury) are distinguished from those having a traumatic brain injury, a mild traumatic brain injury, a moderate traumatic brain injury, a severe traumatic brain injury or a moderate to severe traumatic brain injury. But at the same time, raising the cutoff decreases the number of cases identified as positive overall, as well as the number of true positives, so the sensitivity must decrease. Conversely, the lower the cutoff, sensitivity improves as more true positives (i.e., subjects having a traumatic brain injury, having a mild traumatic brain injury, having a moderate traumatic brain injury, having a severe traumatic brain injury or having a moderate to severe traumatic brain injury) are distinguished from those who do not have a traumatic brain injury, a mild traumatic brain injury, a moderate traumatic brain injury, a severe traumatic brain injury or a moderate to severe traumatic brain injury. But at the same time, lowering the cutoff increases the number of cases identified as positive overall, as well as the number of false positives, so the specificity must decrease.

Generally, a high sensitivity value helps one of skill rule out disease or condition (such as a traumatic brain injury, mild traumatic brain injury, moderate traumatic brain injury, severe traumatic brain injury or moderate to severe traumatic brain injury), and a high specificity value helps one of skill rule in disease or condition. Whether one of skill desires to rule out or rule in disease depends on what the consequences are for the patient for each type of error. Accordingly, one cannot know or predict the precise balancing employed to derive a test cutoff without full disclosure of the underlying information on how the value was selected. The balancing of sensitivity against specificity and other factors will differ on a case-by-case basis. This is why it is sometimes preferable to provide alternate cutoff (e.g., reference) values so a physician or practitioner can choose.

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“Digital assay” as used herein refers to an assay in which an analyte is captured and a molecule of the analyte segregated and interrogated (e.g., to detect the presence and/or amount of the analyte in a sample). In a digital assay, noise is separated from signal. In a digital assay, the results are assigned a value of 1 or 0. Examples of digital assays include one or more of the following (which may overlap but are not mutually exclusive): single molecule detection assay, a nanowell assay, a single molecule enzyme linked immunosorbent assay, a direct capture counting assay, etc.

“Drugs of abuse” is used herein to refer to one or more additive substances (such as a drug) taken for non-medical reasons (such as for, example, recreational and/or mind-altering effects). Excessive overindulgence, use or dependence of such drugs of abuse is often referred to as “substance abuse”. Examples of drugs of abuse include alcohol, barbiturates, benzodiazepines, cannabis, cocaine, hallucinogens (such as ketamine, mescaline (peyote), PCP, psilocybin, DMT and/or LSD), methaqualone, opioids, amphetamines (including methamphetamines), anabolic steroids, inhalants (namely, substances which contain volatile substances that contain psychoactive properties such as, for example, nitrites, spray paints, cleaning fluids, markers, glues, etc.) and combinations thereof.

“Dual-specific antibody” is used herein to refer to a full-length antibody that can bind two different antigens (or epitopes) in each of its two binding arms (a pair of HC/LC) (see PCT publication WO 02/02773). Accordingly, a dual-specific binding protein has two identical antigen binding arms, with identical specificity and identical CDR sequences, and is bivalent for each antigen to which it binds.

“Dual variable domain” is used herein to refer to two or more antigen binding sites on a binding protein, which may be divalent (two antigen binding sites), tetravalent (four antigen binding sites), or multivalent binding proteins. DVDs may be monospecific, i.e., capable of binding one antigen (or one specific epitope), or multispecific, i.e., capable of binding two or more antigens (i.e., two or more epitopes of the same target antigen molecule or two or more epitopes of different target antigens). A preferred DVD binding protein comprises two heavy chain DVD polypeptides and two light chain DVD polypeptides and is referred to as a “DVD immunoglobulin” or “DVD-Ig.” Such a DVD-Ig binding protein is thus tetrameric and reminiscent of an IgG molecule but provides more antigen binding sites than an IgG molecule. Thus, each half of a tetrameric DVD-Ig molecule is reminiscent of one half of an IgG molecule and comprises a heavy chain DVD polypeptide and a light chain DVD polypeptide, but unlike a pair of heavy and light chains of an IgG molecule that provides a single antigen binding domain, a pair of heavy and light chains of a DVD-Ig provide two or more antigen binding sites.

Each antigen binding site of a DVD-Ig binding protein may be derived from a donor (“parental”) monoclonal antibody and thus comprises a heavy chain variable domain (VH) and a light chain variable domain (VL) with a total of six CDRs involved in antigen binding per antigen binding site. Accordingly, a DVD-Ig binding protein that binds two different epitopes (i.e., two different epitopes of two different antigen molecules or two different epitopes of the same antigen molecule) comprises an antigen binding site derived from a first parental monoclonal antibody and an antigen binding site of a second parental monoclonal antibody.

A description of the design, expression, and characterization of DVD-Ig binding molecules is provided in PCT Publication No. WO 2007/024715, U.S. Pat. No. 7,612,181, and Wu et al., Nature Biotech., 25:1290-1297 (2007). A preferred example of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, but preferably 1; and a light chain that comprises the structural formula VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, but preferably 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

In a preferred embodiment, a DVD-Ig binding protein not only binds the same target molecules bound by its parental monoclonal antibodies, but also possesses one or more desirable properties of one or more of its parental monoclonal antibodies. Preferably, such an additional property is an antibody parameter of one or more of the parental monoclonal antibodies. Antibody parameters that may be contributed to a DVD-Ig binding protein from one or more of its parental monoclonal antibodies include, but are not limited to, antigen specificity, antigen affinity, potency, biological function, epitope recognition, protein stability, protein solubility, production efficiency, immunogenicity, pharmacokinetics, bioavailability, tissue cross reactivity, and orthologous antigen binding.

A DVD-Ig binding protein binds at least one epitope of UCH-L1, GFAP, CK-MB β-hCG, TSH, homocysteine, or free T4. Non-limiting examples of a DVD-Ig binding protein include a DVD-Ig binding protein that binds one or more epitopes of UCH-L1, GFAP, CK-MB β-hCG, TSH, homocysteine, or free T4, a DVD-Ig binding protein that binds an epitope of a human UCH-L1, GFAP, CK-MB β-hCG, TSH, homocysteine, or free T4 and an epitope of UCH-L1, GFAP, CK-MB β-hCG, TSH, homocysteine, or free T4 of another species (for example, mouse), and a DVD-Ig binding protein that binds an epitope of a human UCH-L1, GFAP, CK-MB β-hCG, TSH, homocysteine, or free T4 and an epitope of another target molecule.

“Dynamic range” as used herein refers to range over which an assay readout is proportional to the amount of target molecule or analyte in the sample being analyzed.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Fragment antigen-binding fragment” or “Fab fragment” as used herein refers to a fragment of an antibody that binds to antigens and that contains one antigen-binding site, one complete light chain, and part of one heavy chain. Fab is a monovalent fragment consisting of the VL, VH, CL and CH1 domains. Fab is composed of one constant and one variable domain of each of the heavy and the light chain. The variable domain contains the paratope (the antigen-binding site), comprising a set of complementarity determining regions, at the amino terminal end of the monomer. Each arm of the Y thus binds an epitope on the antigen. Fab fragments can be generated such as has been described in the art, e.g., using the enzyme papain, which can be used to cleave an immunoglobulin monomer into two Fab fragments and an Fc fragment, or can be produced by recombinant means.

“F(ab′)₂ fragment” as used herein refers to antibodies generated by pepsin digestion of whole IgG antibodies to remove most of the Fc region while leaving intact some of the hinge region. F(ab′)₂ fragments have two antigen-binding F(ab) portions linked together by disulfide bonds, and therefore are divalent with a molecular weight of about 110 kDa. Divalent antibody fragments (F(ab′)₂ fragments) are smaller than whole IgG molecules and enable a better penetration into tissue thus facilitating better antigen recognition in immunohistochemistry. The use of F(ab′)₂ fragments also avoids unspecific binding to Fc receptor on live cells or to Protein A/G. F(ab′)₂ fragments can both bind and precipitate antigens.

“Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base (hypertext transfer protocol://vbase.mrc-cpe.cam.ac.uk/) or in the international ImMunoGeneTics® (IMGT®) information system (hypertext transfer protocol://imgt.cines.fr/texts/IMGTrepertoire/LocusGenes/).

“Functional antigen binding site” as used herein may mean a site on a binding protein (e.g., an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

“GFAP” is used herein to describe glial fibrillary acidic protein. GFAP is a protein that is encoded by the GFAP gene in humans, and which can be produced (e.g., by recombinant means, in other species).

“GFAP status” can mean either the level or amount of GFAP at a point in time (such as with a single measure of GFAP), the level or amount of GFAP associated with monitoring (such as with a repeat test on a subject to identify an increase or decrease in GFAP amount), the level or amount of GFAP associated with treatment for TBI (whether a primary brain injury and/or a secondary brain injury) or combinations thereof. “Glasgow Coma Scale” or “GCS” as used herein refers to a 15-point scale (e.g., described in 1974 by Graham Teasdale and Bryan Jennett, Lancet 1974; 2:81-4) that provides a practical method for assessing impairment of conscious level in patients who have suffered a brain injury. The test measures the best motor response, verbal response and eye opening response with these values: I. Best Motor Response (6—obey 2—part request; 5—brings hand above clavicle to stimulus on head neck; 4—bends arm at elbow rapidly but features not predominantly abnormal; 3—bends arm at elbow, features clearly predominantly abnormal; 2—extends arm at elbow; 1—no movement in arms/legs, no interfering factor; NT—paralyzed or other limiting factor); II. Verbal Response (5—correctly gives name, place and date; 4—not orientated but communication coherently; 3—intelligible single words; 2—only moans/groans; 1—no audible response, no interfering factor; NT-factor interfering with communication); and III. Eye Opening (4—open before stimulus; 3—after spoken or shouted request; 2—after fingertip stimulus; 1—no opening at any time, no interfering factor; NT—closed by local factor). The final score is determined by adding the values of I+II+III. A subject is considered to have a mild TBI if the GCS score is 13-15. A subject is considered to have a moderate TBI if the GCS score is 9-12. A subject is considered to have a severe TBI if the GCS score is 8 or less, typically 3-8.

“Glasgow Outcome Scale” as used herein refers to a global scale for functional outcome frequently employed in a potential TBI situation that rates patient status into one of five categories: Dead, Vegetative State, Severe Disability, Moderate Disability or Good Recovery. “Extended Glasgow Outcome Scale” or “GOSE” as used interchangeably herein provides more detailed categorization into eight categories by subdividing the categories of severe disability, moderate disability and good recovery into a lower and upper category as shown in Table 1.

TABLE 1

| | | | |
|---|---|---|---|
| 1 | Death | D | |
| 2 | Vegetative state | VX | |
| 3 | Lower severe | SD− | Condition of unawareness with only reflex |
| | disability | | responses but with periods of spontaneous |
| 4 | Upper severe | SD+ | eye opening |
| | disability | | |
| 5 | Lower moderate | MD− | Patient who is dependent for daily support |
| | disability | | for mental or physical disability, usually a |
| 6 | Upper moderate | MD+ | combination of both. If the patient can be |
| | disability | | left alone for more than 8 hours at home it is |
| | | | upper level of SD, if not then it is low level |
| | | | of SD. |
| 7 | Lower good | GR− | Patients have some disability such as |
| | recovery | | aphasia, hemiparesis or epilepsy and/or |
| 8 | Upper good | GR+ | deficits of memory or personality but are |
| | recovery | | able to look after themselves. They are |
| | | | independent at home but dependent outside. |
| | | | If they are able to return to work even with |
| | | | special arrangement it is upper level of MD, |
| | | | if not then it is low level of MD. |

“Higher throughput assay analyzer” or a “non-point-of-care device”, as used interchangeably herein, refers to a device that is not a point-of-care device or a single use device. A higher throughput assay analyzer or non-point-of-care device refers to any device that does not meet any of the limitations of a point-of-care or a single use device as defined herein. In some embodiments, a “higher throughput assay analyzer” or “non-point-of-care device” refers to an instrument that: (a) may be a relatively large instrument compared to a hand-held point-of-care device, e.g., such as ranging in size from that of a tabletop instrument (e.g., typically considered low- or medium-throughput) to a large room-size or multiple-room-size instrument (e.g., typically considered high throughput); (b) is not a handheld instrument; (c) is capable of performing an assay on more than one clinical sample simultaneously; and (d) any combination of (a)-(c). A higher throughput assay analyzer may be a clinical chemistry analyzer, an immunoassay analyzer, or a combination thereof. Exemplary higher throughput assay analyzers or non-point-of-care devices include, for example, the ARCHITECT or Alinity platforms produced by Abbott Laboratories.]

“Homocysteine” or “HCY” refers to a thiol-containing amino acid produced by the intracellular demethylation of methionine. Homocysteine is exported into plasma where it circulates, mostly in its oxidized form, bound to plasma proteins as a protein-HCY mixed disulfide with albumin. Smaller amounts of reduced homocysteine and the disulfide homocystine (HCY—SS—HCY) are present. Total homocysteine (tHCY) represents the sum of all homocysteine species found in serum or plasma (free plus protein bound). Homocysteine is metabolized to either cysteine or methionine. In the vitamin B6 dependent trans-sulphuration pathway, homocysteine is irreversibly catabolized to cysteine. A major part of homocysteine is remethylated to methionine, mainly by the folate and cobalamin dependent enzyme methionine synthase. Homocysteine accumulates and is excreted into the blood when these reactions are impaired. Impaired homocysteine metabolism results in hyperhomocysteinemia (increased levels of homocysteine in plasma or serum) or homocystinuria (high plasma levels cause homocysteine to be excreted in urine). Hyperhomocysteinemia is caused by nutritional and genetic deficiencies. The majority of elevated homocysteine cases (two/thirds) in the general population are due to deficiency of folic acid, vitamin B6 and vitamin B12. Severely elevated concentrations of total homocysteine are found in subjects with homocystinuria, a rare genetic disorder of the enzymes involved in the metabolism of homocysteine. Patients with homocystinuria exhibit mental retardation, early arteriosclerosis and arterial and venous thromboembolism. Other less severe genetic defects which lead to moderately elevated levels of total homocysteine are also found. Studies have investigated the relationship between elevated homocysteine concentrations and cardiovascular disease (CVD), indicating homocysteine as an important marker for risk assessment. In the presence of known coronary artery disease (CAD), it has been shown to be a strong independent marker of subsequent CAD-related death. In intermediate risk patients, elevated homocysteine levels are associated with the quantity of coronary artery calcification. Elevated homocysteine levels in these patients are independent of coronary heart disease (CHD) risk factors.

It has been suggested that elevated homocysteine is a modifiable, independent risk factor for CAD, stroke and deep vein thrombosis. Studies have also identified elevated homocysteine as a strong independent risk factor for developing various forms of dementia, including Alzheimer's Disease. Increased tHCY is associated with increased risk of pregnancy complications (preeclampsia, recurrent early pregnancy loss, premature delivery, low birth weight, and placental abruption or infarction). Maternal hyperhomocysteinemia is related to birth defects such as neural tube defects, orofacial clefts, club foot and Down's Syndrome.

“Human Thyroid Stimulating Hormone” or “TSH” refers to a glycoprotein with a molecular weight of approximately 28,000 daltons, synthesized by the basophilic cells (thyrotropes) of the anterior pituitary. TSH is composed of two non-covalently linked subunits designated alpha and beta. Although the alpha subunit of TSH is common to the luteinizing hormone (LH), follicle stimulating hormone (FSH) and human chorionic gonadotropin (hCG), the beta subunits of these glycoproteins are hormone specific and confer biological as well as immunological specificity. Both alpha and beta subunits are required for biological activity. TSH stimulates the production and secretion of the metabolically active thyroid hormones, thyroxine (T4) and triiodothyronine (T3), by interacting with a specific receptor on the thyroid cell surface. T3 and T4 are responsible for regulating diverse biochemical processes throughout the body which are essential for normal development and metabolic and neural activity. The synthesis and secretion of TSH is stimulated by thyrotropin releasing hormone (TRH), the hypothalamic tripeptide, in response to low levels of circulating thyroid hormones. Elevated levels of T3 and T4 suppress the production of TSH via a classic negative feedback mechanism. Other evidence also indicates that somatostatin and dopamine exert inhibitory control over TSH release, suggesting that the hypothalamus may provide both inhibitory and stimulatory influence on pituitary TSH production. Failure at any level of regulation of the hypothalamic-pituitary-thyroid axis will result in either underproduction (hypothyroidism) or overproduction (hyperthyroidism) of T4 and/or T3.

In cases of primary hypothyroidism, T3 and T4 levels are low and TSH levels are significantly elevated. In the case of pituitary dysfunction, either due to intrinsic hypothalamic or pituitary disease; i.e., central hypothyroidism, normal or marginally elevated basal TSH levels are often seen despite significant reduction in T4 and/or T3 levels. These inappropriate TSH values are due to a reduction in TSH bioactivity which is frequently observed in such cases. Routine TRH stimulation is advised to confirm the diagnosis in such cases. Secondary hypothyroidism typically results in an impaired TSH response to TRH, while in tertiary hypothyroidism the TSH response to TRH may be normal, prolonged or exaggerated. Primary hyperthyroidism (e.g., Grave's Disease, nodular goiter) is associated with high levels of thyroid hormones and depressed or undetectable levels of TSH. The TRH stimulation test has been used in diagnosis of hyperthyroidism. Hyperthyroid patients show a subnormal response to the TRH test. In addition, large doses of glucocorticoids, somatostatin, dopamine and replacement doses of thyroid hormones reduce or totally blunt the TSH response to TRH.

As used herein the term “hydrophilic”, such as in reference to a “hydrophilic material” (e.g., membrane, film, etc.) refers to those materials having a water contact angle of less than about 40 degrees.

As used herein the term “hydrophobic”, such as in reference to a “hydrophobic material” (e.g., membrane, film, etc.) refers to those materials having a water contact angle greater than about 80 degrees.

“Humanized antibody” is used herein to describe an antibody that comprises heavy and light chain variable region sequences from a non-human species (e.g., a mouse) but in which at least a portion of the VH and/or VL sequence has been altered to be more “human-like,” i.e., more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98%, or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well-known in the art.

The framework regions and CDRs of a humanized antibody need not correspond precisely to the parental sequences, e.g., the donor antibody CDR or the consensus framework may be mutagenized by substitution, insertion, and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, 1987)). A “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence.

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

“Injury to the head” or “head injury” as used interchangeably herein, refers to any trauma to the scalp, skull, or brain. Such injuries may include only a minor bump on the skull or may be a serious brain injury. Such injuries include primary injuries to the brain and/or secondary injuries to the brain. Primary brain injuries occur during the initial insult and result from displacement of the physical structures of the brain. More specifically, a primary brain injury is the physical damage to parenchyma (tissue, vessels) that occurs during the traumatic event, resulting in shearing and compression of the surrounding brain tissue. Secondary brain injuries occur subsequent to the primary injury and may involve an array of cellular processes. More specifically, a secondary brain injury refers to the changes that evolve over a period of time (from hours to days) after the primary brain injury. It includes an entire cascade of cellular, chemical, tissue, or blood vessel changes in the brain that contribute to further destruction of brain tissue.

An injury to the head can be either closed or open (penetrating). A closed head injury refers to a trauma to the scalp, skull or brain where there is no penetration of the skull by a striking object. An open head injury refers a trauma to the scalp, skull or brain where there is penetration of the skull by a striking object. An injury to the head may be caused by physical shaking of a person, by blunt impact by an external mechanical or other force that results in a closed or open head trauma (e.g., vehicle accident such as with an automobile, plane, train, etc.; blow to the head such as with a baseball bat, or from a firearm), a cerebral vascular accident (e.g., stroke), one or more falls (e.g., as in sports or other activities), explosions or blasts (collectively, “blast injuries”) and by other types of blunt force trauma. Alternatively, an injury to the head may be caused by the ingestion and/or exposure to a fire, chemical, toxin or a combination of a chemical and toxin. Examples of such chemicals and/or toxins include molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. Alternatively, an injury to the head may be caused as a result of a subject suffering from an autoimmune disease, a metabolic disorder, a brain tumor, hypoxia, a viral infection (e.g., SARS-COV-2), a fungal infection, a bacterial infection, meningitis, hydrocephalus, or any combinations thereof. In some cases, it is not possible to be certain whether any such event or injury has occurred or taken place. For example, there may be no history on a patient or subject, the subject may be unable to speak, the subject may be aware of what events they were exposed to, etc. Such circumstances are described herein as the subject “may have sustained an injury to the head.” In certain embodiments herein, the closed head injury does not include and specifically excludes a cerebral vascular accident, such as stroke.

“Interstitial fluid” as used herein refers to the fluid that surrounds and/or fills the space between cells. Interstitial fluid can contain or comprise a mixture of water, ions, and small solutes that are forced out of the blood by the systolic pressure created when the heart pumps.

“Intracranial lesion” as used herein refers to an area of injury within the brain. An intracranial lesion can be an abnormality seen on a CT scan or brain-imaging test, such as magnetic resonance imaging (MRI). On CT or MRI scans, brain lesions can appear as dark or light spots that do not look like normal brain tissue.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as 3H, 14C, 32P, 33P, 35S, 90Y, 99Tc, 111In, 125I, 131I, 177Lu, 166Ho, and 153Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immunopolymerase chain reaction label. An introduction to labels, labeling procedures and detection of labels is found in Polak and Van Noorden, Introduction to Immunocytochemistry, 2^{nd} ed., Springer Verlag, N.Y. (1997), and in Haugland, Handbook of Fluorescent Probes and Research Chemicals (1996), which is a combined handbook and catalogue published by Molecular Probes, Inc., Eugene, Oregon. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16:1324-1328 (2006); Adamczyk et al., Bioorg. Med. Chem. Lett. 4:2313-2317 (2004); Adamczyk et al., Biorg. Med. Chem. Lett. 14:3917-3921 (2004); and Adamczyk et al., Org. Lett. 5:3779-3782 (2003)).

In one aspect, the acridinium compound is an acridinium-9-carboxamide. Methods for preparing acridinium 9-carboxamides are described in Mattingly, J. Biolumin. Chemilumin. 6:107-114 (1991); Adamczyk et al., J. Org. Chem. 63:5636-5639 (1998); Adamczyk et al., Tetrahedron 55:10899-10914 (1999); Adamczyk et al., Org. Lett. 1:779-781 (1999); Adamczyk et al., Bioconjugate Chem. 11:714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5:3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl) acridinium fluorosulfonate (available from Cayman Chemical, Ann Arbor, MI). Methods for preparing acridinium 9-carboxylate aryl esters are described in McCapra et al., Photochem. Photobiol. 4:1111-21 (1965); Razavi et al., Luminescence 15:245-249 (2000); Razavi et al., Luminescence 15:239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

“Linking sequence” or “linking peptide sequence” refers to a natural or artificial polypeptide sequence that is connected to one or more polypeptide sequences of interest (e.g., full-length, fragments, etc.). The term “connected” refers to the joining of the linking sequence to the polypeptide sequence of interest. Such polypeptide sequences are preferably joined by one or more peptide bonds. Linking sequences can have a length of from about 4 to about 50 amino acids. Preferably, the length of the linking sequence is from about 6 to about 30 amino acids. Natural linking sequences can be modified by amino acid substitutions, additions, or deletions to create artificial linking sequences. Linking sequences can be used for many purposes, including in recombinant Fabs. Exemplary linking sequences include, but are not limited to: (i) Histidine (His) tags, such as a 6X His tag, which has an amino acid sequence of HHHHHH (SEQ ID NO: 3), are useful as linking sequences to facilitate the isolation and purification of polypeptides and antibodies of interest; (ii) Enterokinase cleavage sites, like His tags, are used in the isolation and purification of proteins and antibodies of interest. Often, enterokinase cleavage sites are used together with His tags in the isolation and purification of proteins and antibodies of interest. Various enterokinase cleavage sites are known in the art. Examples of enterokinase cleavage sites include, but are not limited to, the amino acid sequence of DDDDK (SEQ ID NO: 4) and derivatives thereof (e.g., ADDDDK (SEQ ID NO: 5), etc.); (iii) Miscellaneous sequences can be used to link or connect the light and/or heavy chain variable regions of single chain variable region fragments. Examples of other linking sequences can be found in Bird et al., Science 242:423-426 (1988); Huston et al., PNAS USA 85:5879-5883 (1988); and McCafferty et al., Nature 348:552-554 (1990). Linking sequences also can be modified for additional functions, such as attachment of drugs or attachment to solid supports. In the context of the present disclosure, the monoclonal antibody, for example, can contain a linking sequence, such as a His tag, an enterokinase cleavage site, or both.

“Magnetic resonance imaging” or “MRI” as used interchangeably herein refers to a medical imaging technique used in radiology to form pictures of the anatomy and the physiological processes of the body in both health and disease (e.g., referred to herein interchangeably as “an MRI”, “an MRI procedure” or “an MRI scan”). MRI is a form of medical imaging that measures the response of the atomic nuclei of body tissues to high-frequency radio waves when placed in a strong magnetic field, and that produces images of the internal organs. MRI scanners, which is based on the science of nuclear magnetic resonance (NMR), use strong magnetic fields, radio waves, and field gradients to generate images of the inside of the body.

As used herein, the term “microchannel” refers to a channel having a cross-sectional dimension (namely, height and width) that is less than about 200 μm. In some aspects, the channel has a cross-sectional dimension of less than about 150 μm. In yet other aspects, the channel has a cross-sectional dimension of less than about 100 μm.

“Microsampling device” as used herein refers to any device known in the art that is suitable for extracting capillary blood through the skin. It is understood that while a sample obtained through the skin using a microsampling device will comprise predominantly capillary blood, the sample may also comprise a small amount or percentage of interstitial fluid. In some aspects, the microsampling device can comprise from about 0.1 mL to about 4 mL of capillary blood. In some other aspects, the device contains a plurality of microneedles, lancets or microlancets, blades or microblades, microscrews, or any combination thereof. In some aspects, the plurality of microneedles, lancets or microlancets, blades or microblades, microscrews, or any combination thereof can be rotating. In yet other aspects, the plurality of microneedles, lancets or microlancets, blades or microblades, microscrews, or any combination thereof are non-rotating. In some aspects, the microsampling device creates a vacuum and/or uses a stored vacuum to pull the skin into the device and/or activate the plurality of microneedles, lancets or microlancets, blades or microblades, microscrews, or any combination thereof to cut the skin. Examplary microsampling devices which can be used in the methods described herein include the TAP device available from YourBio Health, Inc. (Cambridge, MA) as well as the device described in U.S. Pat. No. 9,113,836, the contents of which are herein incorporated by reference, the Tasso+, Tasso-M20, and Tasso-ST devices available from Tasso, Inc. (Seattle, WA), the One Draw device available from Draw Bridge Health (San Diego, CA), PBS-1000 from PreciHealth (Neuchatel, Switzerland) or the Loop blood collection device available from Loop Medical (Lausanne, Switzerland). In other aspects, an example of a microsampling device includes a fingerstick device. In some aspects, the microsampling device can include a band-aid, bandage, or other suitable material which can be applied or dispensed to the area of the skin once the sample is obtained and/or the device is removed and/or detached from the skin.

“Monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological.

“Multivalent binding protein” is used herein to refer to a binding protein comprising two or more antigen binding sites (also referred to herein as “antigen binding domains”). A multivalent binding protein is preferably engineered to have three or more antigen binding sites and is generally not a naturally occurring antibody. The term “multispecific binding protein” refers to a binding protein that can bind two or more related or unrelated targets, including a binding protein capable of binding two or more different epitopes of the same target molecule.

“Negative predictive value” or “NPV” as used interchangeably herein refers to the probability that a subject has a negative outcome given that they have a negative test result.

“Normalize” or “normalizing” as used herein refers adjusting the amount of an analyte (e.g., UCH-L1, GFAP, CK-MB, β-hCG, TSH, homocysteine, and/or free T4) determined in a capillary blood sample obtained from a subject based on the amount of the same analyte in venous blood. In some aspects, for example, normalizing can involve multiplying a factor (e.g., correlation or conversion factor) by the amount of the analyte in the capillary blood sample. As described herein, a “conversion factor” generally is in the form of a conversion factor being described for (i) an analyte in a capillary sample (i.e., capillary whole blood sample) compared to the same analyte in a venous sample (i.e., venous whole blood sample); or (ii) an analyte in a venous sample (i.e., venous whole blood sample) compared to the same analyte in a capillary sample (i.e., capillary whole blood sample). Theoretically, the value of the conversion factor should be comparable for the conversion factor obtained for a capillary plasma sample to a venous plasma sample (e.g., depending on the efficiency of plasma separation, and other factors as would be apparent to those skilled in the art).

“Operatively coupled” or “operatively linked” as used herein means that a number of elements or assemblies, each of which is movable between a first position and a second position, or a first configuration and a second configuration, are coupled so that as the first element moves from one position/configuration to the other, the second element moves between positions/configurations as well. It is noted that a first element may be “operatively coupled” to another without the opposite being true. Where movement is capable between a first element and another element, and vice versa, the elements are said to be “reciprocally operatively coupled”.

“Point-of-care device” refers to a device used to provide medical diagnostic testing at or near the point-of-care (namely, typically, outside of a laboratory), at the time and place of patient care (such as in a hospital, physician's office, urgent or other medical care facility, a patient's home, a nursing home and/or a long-term care and/or hospice facility). Examples of point-of-care devices include those produced by Abbott Laboratories (Abbott Park, IL) (e.g., i-STAT and i-STAT Alinity, Universal Biosensors (Rowville, Australia) (see US 2006/0134713), Axis-Shield PoC AS (Oslo, Norway) and Clinical Lab Products (Los Angeles, USA).

“Positive predictive value” or “PPV” as used interchangeably herein refers to the probability that a subject has a positive outcome given that they have a positive test result.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the amount of an analyte, such as an antibody or an analyte. Alternatively, a single calibrator, which is near a reference level or control level (e.g., “low”, “medium”, or “high” levels), can be used. Multiple calibrators (i.e., more than one calibrator or a varying amount of calibrator(s)) can be used in conjunction to comprise a “sensitivity panel.”

As used herein, a “reaction vessel” refers to a holder or receiver, such as a container, receptacle, tube, and/or cartridge, in or upon which an assay is performed. In some aspects, a reaction vessel may have one or more apertures.

A “receiver operating characteristic” curve or “ROC” curve refers to a graphical plot that illustrates the performance of a binary classifier system as its discrimination threshold is varied. For example, a ROC curve can be a plot of the true positive rate against the false positive rate for the different possible cutoff points of a diagnostic test. It is created by plotting the fraction of true positives out of the positives (TPR=true positive rate) vs. the fraction of false positives out of the negatives (FPR=false positive rate), at various threshold settings. TPR is also known as sensitivity, and FPR is one minus the specificity or true negative rate. The ROC curve demonstrates the tradeoff between sensitivity and specificity (any increase in sensitivity will be accompanied by a decrease in specificity); the closer the curve follows the left-hand border and then the top border of the ROC space, the more accurate the test; the closer the curve comes to the 45-degree diagonal of the ROC space, the less accurate the test; the slope of the tangent line at a cutoff point gives the likelihood ratio (LR) for that value of the test; and the area under the curve is a measure of test accuracy.

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-Ig®s, and other antibodies as described in (i) herein. (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e., the bifunctional antibodies have a dual specificity.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). An “absolute amount” as used herein refers to the absolute value of a change or difference between at least two assay results taken or sampled at different time points and, which similar to a reference level, has been linked or is associated herein with various clinical parameters (e.g., presence of disease, stage of disease, severity of disease, progression, non-progression, or improvement of disease, etc.). “Absolute value” as used herein refers to the magnitude of a real number (such as, for example, the difference between two compared levels (such as levels taken at a first time point and levels taken at a second time point)) without regard to its sign, i.e., regardless of whether it is positive or negative. The UCH-L1 and/or GFAP or CK-MB, β-hCG, TSH, homocysteine and/or free T4 reference levels referred to herein are from venous blood.

This disclosure provides exemplary reference levels and absolute amounts (e.g., calculated by comparing reference levels at different time points). However, it is well-known that reference levels and absolute amounts may vary depending on the nature of the immunoassay (e.g., antibodies employed, reaction conditions, sample purity, etc.) and that assays can be compared and standardized. It further is well within the ordinary skill of one in the art to adapt the disclosure herein for other immunoassays to obtain immunoassay-specific reference levels and absolute amounts for those other immunoassays based on the description provided by this disclosure. Whereas the precise value of the reference level and absolute amount may vary between assays, the findings as described herein should be generally applicable and capable of being extrapolated to other assays.

“Removably coupled” or “removably linked” as used herein means that one component is coupled with another component in an essentially temporary manner. That is, the two components are coupled in such a way that the joining or separation of the components is easy and does not damage the components. Accordingly, “removably coupled” components may be readily uncoupled and recoupled without damage to the components.

“Result” as used herein refers to an item of information obtained by performing an assay. In one aspect, a result is an amount of a biomarker (e.g., UCH-L1, GFAP, CK-MB, β-hCG, TSH, homocysteine, free T4, or any combinations thereof) in a test sample (e.g, capillary blood sample). In another aspect, a result is identifying the presence of biomarker (e.g., UCH-L1, GFAP, CK-MB, β-hCG, TSH, homocysteine, free T4or any combinations thereof) in a sample. A result can be visually displayed (e.g., as a readout).

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Plasma separation device” as used herein, refers to an apparatus or device that can be used to separate components of whole blood (e.g., red and white blood cells) from serum, plasma or serum and plasma using a separation system, such as, for example, at least one membrane, filter, synthetic paper (e.g., micropillar scaffolds) or any combination thereof. For example, the membrane and/or filter that can be used in the plasma separation device may comprise at least one of polycarbonate, polysulfone, polyester, polyethylene, polyurethane and polypropylene. In some aspects, the membrane and/or filter is pre-treated (e.g., with one or more polycations, zwitterions, one or more noncovalent surface treatments (e.g., PEGMA, HEMA, BSA, O₂ plasma etc.)). In other aspects, the membrane and/or filter is not pre-treated. In still further aspects, the filter that can be used is a gravity-assisted separation system. Examples of plasma separation devices that can be used in the methods described herein include those described in U.S. Patent Publication No. 2020/0124508, the contents of which are herein incorporated by reference. In some aspects, a plasma separation device does not include a lateral flow device.

“Sensitivity” of an assay as used herein refers to the proportion of subjects for whom the outcome is positive that are correctly identified as positive (e.g., correctly identifying those subjects with a disease or medical condition for which they are being tested). For example, this might include correctly identifying subjects as having a TBI as distinct from those who do not have a TBI, correctly identifying subjects having a moderate, severe, or moderate to severe TBI as distinct from those having a mild TBI, correctly identifying subjects as having a mild TBI as distinct from those having a moderate, severe, or moderate to severe TBI, correctly identifying subjects as having a moderate, severe, or moderate to severe TBI as distinct from those having no TBI or correctly identifying subjects as having a mild TBI as distinct from those having no TBI etc.

“Specificity” of an assay as used herein refers to the proportion of subjects for whom the outcome is negative that are correctly identified as negative (e.g., correctly identifying those subjects who do not have a disease or medical condition for which they are being tested). For example, this might include correctly identifying subjects not having an TBI as distinct from those who do have a TBI, correctly identifying subjects not having a moderate, severe, or moderate to severe TBI as distinct from those having a mild TBI, correctly identifying subjects as not having a mild TBI as distinct from those having a moderate, severe, or moderate to severe TBI, etc.).

“Series of calibrating compositions” refers to a plurality of compositions comprising a known amount of UCH-L1, GFAP, UCH-L1, CK-MB, β-hCG, TSH, homocysteine, or free T4 wherein each of the compositions differs from the other compositions in the series by the amount of UCH-L1, GFAP, UCH-L1, CK-MB, β-hCG, TSH, homocysteine, or free T4.

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Statistically significant” as used herein refers to the likelihood that a relationship between two or more variables is caused by something other than random chance. Statistical hypothesis testing is used to determine whether the result of a data set is statistically significant. In statistical hypothesis testing, a statistically significant result is attained whenever the observed p-value of a test statistic is less than the significance level defined of the study. The p-value is the probability of obtaining results at least as extreme as those observed, given that the null hypothesis is true. Examples of statistical hypothesis analysis include Wilcoxon signed-rank test, t-test, Chi-Square or Fisher's exact test. “Significant” as used herein refers to a change that has not been determined to be statistically significant (e.g., it may not have been subject to statistical hypothesis testing).

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject may be a human or a non-human. In some embodiments, the subject is a human. The subject or patient may be undergoing other forms of treatment.

“Thyroxine” or “T4” circulates in the blood as an equilibrium mixture of free and serum protein bound hormone. Thyroxine binding globulin (TBG), albumin and pre-albumin bind approximately 75%, 10% and 15% of the total circulating T4 respectively. The binding of T4 by these proteins is such that less than 0.03% is present in the circulation as unbound, free T4. This small percentage of the total T4 represents the physiologically available hormone which is biologically active. Once the free T4 is absorbed by the target cells, the equilibrium reestablishes circulating free T4 levels. The equilibrium results in the maintenance of a constant level of free T4 when alterations occur in either the concentration or affinity of the serum binding proteins. Therefore, in a variety of normal (pregnancy) and abnormal (Familial Dysalbuminemic Hyperthyroxinemia, FDH) states, or as a result of the administration of certain drugs (e.g., furosemide and fenclofenac), the target tissues are assured of receiving the required amount of hormone. Free T4 values often provided the best indication of thyroid dysfunction, since free T4 is less sensitive to changes in the serum binding proteins.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

As used herein, a “transfer tube” refers to a container or receptacle used to transfer a fluid (e.g., a capillary blood sample) from one location to a second location (e.g., to a reaction vessel or from a plasma separation device).

“Traumatic Brain Injury” or “TBI” as used interchangeably herein refers to a complex injury with a broad spectrum of symptoms and disabilities. TBI is most often an acute event similar to other injuries. TBI can be classified as “mild,” “moderate,” or “severe.” The causes of TBI are diverse and include, for example, physical shaking by a person, a car accident, injuries from firearms, cerebral vascular accidents (e.g., strokes), falls, explosions or blasts and other types of blunt force trauma. Other causes of TBI include the ingestion and/or exposure to one or more fires, chemicals or toxins (such as molds, asbestos, pesticides and insecticides, organic solvents, paints, glues, gases (such as carbon monoxide, hydrogen sulfide, and cyanide), organic metals (such as methyl mercury, tetraethyl lead and organic tin), one or more drugs of abuse or combinations thereof). Alternatively, TBI can occur in subjects suffering from an autoimmune disease, a metabolic disorder, a brain tumor, hypoxia, a viral infection (e.g., SARS-COV-2, meningitis, etc.), fungal infection (e.g., meningitis), bacterial infection (e.g., meningitis), or any combinations thereof. Young adults and the elderly are the age groups at highest risk for TBI. In certain embodiments herein, traumatic brain injury or TBI does not include and specifically excludes cerebral vascular accidents such as strokes.

“Mild TBI” as used herein refers to a head injury where a subject may or may not experience a loss of consciousness. For subjects that experience a loss of consciousness, it is typically brief, usually lasting only a few seconds or minutes. Mild TBI is also referred to as a concussion, minor head trauma, minor TBI, minor brain injury, and minor head injury. While MRI and CT scans are often normal, the individual with mild TBI may have cognitive problems such as headache, difficulty thinking, memory problems, attention deficits, mood swings and frustration.

Mild TBI is the most prevalent TBI and is often missed at time of initial injury. Typically, a subject has a Glasgow Coma scale number of between 13-15 (such as 13-15 or 14-15). Fifteen percent (15%) of people with mild TBI have symptoms that last 3 months or more. Common symptoms of mild TBI include fatigue, headaches, visual disturbances, memory loss, poor attention/concentration, sleep disturbances, dizziness/loss of balance, irritability-emotional disturbances, feelings of depression, and seizures. Other symptoms associated with mild TBI include nausea, loss of smell, sensitivity to light and sounds, mood changes, getting lost or confused, and/or slowness in thinking.

“Moderate TBI” as used herein refers to a brain injury where loss of consciousness and/or confusion and disorientation is between 1 and 24 hours and the subject has a Glasgow Coma scale number of between 9-13 (such as 9-12 or 9-13). The individual with moderate TBI may have abnormal brain imaging results. “Severe TBI” as used herein refers to a brain injury where loss of consciousness is more than 24 hours and memory loss after the injury or penetrating skull injury longer than 24 hours and the subject has a Glasgow Coma scale number between 3-8. The deficits range from impairment of higher level cognitive functions to comatose states. Survivors may have limited function of arms or legs, abnormal speech or language, loss of thinking ability or emotional problems. Individuals with severe injuries can be left in long-term unresponsive states. For many people with severe TBI, long-term rehabilitation is often necessary to maximize function and independence.

“Moderate to severe” TBI as used herein refers to a spectrum of brain injury that includes a change from moderate to severe TBI over time and thus encompasses (e.g., temporally) moderate TBI alone, severe TBI alone, and moderate to severe TBI combined. For example, in some clinical situations, a subject may initially be diagnosed as having a moderate TBI but who, over the course of time (minutes, hours or days), progresses to having a severe TBI (such, as for example, in situations when there is a brain bleed). Alternatively, in some clinical situations, a subject may initially be diagnosed as having a severe TBI but who, over the course of time (minutes, hours or days), progresses to having a moderate TBI. Such subjects would be examples of patients that could be classified as “moderate to severe”. Common symptoms of moderate to severe TBI include cognitive deficits including difficulties with attention, concentration, distractibility, memory, speed of processing, confusion, perseveration, impulsiveness, language processing, and/or “executive functions”, not understanding the spoken word (receptive aphasia), difficulty speaking and being understood (expressive aphasia), slurred speech, speaking very fast or very slow, problems reading, problems writing, difficulties with interpretation of touch, temperature, movement, limb position and fine discrimination, the integration or patterning of sensory impressions into psychologically meaningful data, partial or total loss of vision, weakness of eye muscles and double vision (diplopia), blurred vision, problems judging distance, involuntary eye movements (nystagmus), intolerance of light (photophobia), hearing issues, such as decrease or loss of hearing, ringing in the ears (tinnitus), increased sensitivity to sounds, loss or diminished sense of smell (anosmia), loss or diminished sense of taste, the convulsions associated with epilepsy that can be several types and can involve disruption in consciousness, sensory perception, or motor movements, problems with control of bowel and bladder, sleep disorders, loss of stamina, appetite changes, problems with regulation of body temperature, menstrual difficulties, dependent behaviors, issues with emotional ability or stability, lack of motivation, irritability, aggression, depression, disinhibition, or denial/lack of awareness. Subjects having a moderate to severe TBI can have a Glasgow Coma scale score from 3-12 (which includes the range of 9-12 for a moderate TBI, and 3-8 for a severe TBI).

“Ubiquitin carboxy-terminal hydrolase L1” or “UCH-L1” as used interchangeably herein refers to a deubiquitinating enzyme encoded by the UCH-L1 gene in humans. UCH-L1, also known as ubiquitin carboxyl-terminal esterase L1 and ubiquitin thiolesterase, is a member of a gene family whose products hydrolyze small C-terminal adducts of ubiquitin to generate the ubiquitin monomer.

“UCH-L1 status” can mean either the level or amount of UCH-L1 at a point in time (such as with a single measure of UCH-L1), the level or amount of UCH-L1 associated with monitoring (such as with a repeat test on a subject to identify an increase or decrease in UCH-L1 amount), the level or amount of UCH-L1 associated with treatment for traumatic brain injury (whether a primary brain injury and/or a secondary brain injury) or combinations thereof.

“Variant” is used herein to describe a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically reactive fragment of an anti-UCH-L1 antibody that differs from the corresponding fragment of anti-UCH-L1 antibody in amino acid sequence but is still antigenically reactive and can compete with the corresponding fragment of anti-UCH-L1 antibody for binding with UCH-L1. “Variant” also can be used to describe a polypeptide or a fragment thereof that has been differentially processed, such as by proteolysis, phosphorylation, or other post-translational modification, yet retains its antigen reactivity.

“Vector” is used herein to describe a nucleic acid molecule that can transport another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

“Venous blood” as used herein refers to a blood sample that is obtained from the veins from a subject using a syringe, needle, or combination thereof, or any appropriate device. In some embodiments, a venous blood sample is obtained by a trained health clinician such as a physician, phlebotomist, nurse, laboratory technician, or combination thereof. In some embodiments, a venous blood sample is whole blood, serum or plasma.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

2. Methods for Determining an Amount or Presence of UCH-L1, GFAP, or a Combination Thereof in a Capillary Blood Sample Obtained from a Subject

In one embodiment, the present disclosure relates to improved methods for determining an amount (e.g., a quantitative measure) or the presence (e.g., a qualitative measure) of UCH-L1, GFAP, or UCH-L1 and GFAP in a capillary blood sample obtained from a subject (e.g., a human subject). The amount or presence of UCH-L1, GFAP, or UCH-L1 and GFAP determined according to the methods described herein can be used for evaluating and/or aiding in the diagnosis and evaluation of whether a subject (e.g., a human subject) has sustained or may have sustained an injury to the head. The methods and devices described herein can aid in determining the extent of traumatic brain injury in a subject (e.g., human subject) with an actual or suspected injury to the head, e.g., determining whether the subject (e.g., a human subject) has a mild traumatic brain injury, moderate traumatic brain injury, severe traumatic brain injury, or a moderate to severe traumatic brain injury. As used herein, “determining whether the subject (e.g., a human subject) has a mild traumatic brain injury, a moderate traumatic brain injury, a severe traumatic brain injury, or a moderate to severe brain injury” refers to the fact that the aforementioned method can be used, e.g., with other information (e.g., clinical assessment data), to determine that the subject is more likely than not to have a mild traumatic brain injury, moderate traumatic brain injury, severe traumatic brain injury, or moderate to severe traumatic brain injury. The method can include performing an assay on a sample obtained from the subject (e.g., a human subject) within about 24 hours after an actual or suspected injury to the head to measure or detect a level of a biomarker of traumatic brain injury, such as ubiquitin carboxy-terminal hydrolase L1 (UCH-L1), glial fibrillary acidic protein (GFAP), or a combination thereof, in the sample and determining whether the subject (e.g., a human subject) has sustained a mild, moderate, severe, or a moderate to severe traumatic brain injury (TBI). In some embodiments, the subject is determined as having a mild, moderate, severe, or moderate or severe TBI when the amount of the biomarker in the sample is higher than a reference level of a biomarker (e.g., UCH-L1, GFAP, or a combination of UCH-L1 and GFAP).

In some aspects, the sample obtained from a subject (e.g., a human subject) is a capillary blood sample, such as, whole blood, serum or plasma. In some aspects, the capillary blood sample is whole blood. In other aspects, the capillary blood sample is serum. In yet other aspects, the capillary blood sample is plasma.

The capillary blood sample can be obtained by extracting the sample through the skin (e.g., such as the fingers and/or toes, a hand, a foot (including the heel), an earlobe, a location on the arms and/or legs, chest, back, head, or any combinations thereof) of a subject. In some aspects, the capillary blood sample is extracted from the fingers or toes. In other aspects, the whole capillary blood sample is extracted from the arms or legs. In still other aspects, the capillary blood sample is obtained from the hands or feet. In still other aspects, the capillary blood sample is obtained from the chest or back. In yet other aspects, the capillary blood sample is obtained from an earlobe. In still other aspects, the capillary blood sample is obtained from the head.

In some aspects, the capillary blood sample obtained from the subject is obtained without the use of a syringe, needle (e.g., 21-gauge needle, a butterfly needle, etc.), or any other suitable device, or any combination thereof which are typically used to draw blood (e.g., venous blood). Instead, the capillary blood sample is obtained using a self-or other-administered blood collection device. Examples of self-or other-administered blood collection devices include microsampling devices. Example microsampling devices which can be used herein include the TAP device available from YourBio Health, Inc. (Cambridge, MA) as well as the device described in U.S. Pat. No. 9,113,836, the contents of which are herein incorporated by reference, the Tasso+, Tasso-M20, and Tasso-ST devices available from Tasso, Inc. (Seattle, WA), the One Draw device available from Draw Bridge Health (San Diego, CA), PBS-1000 from PreciHealth (Neuchatel, Switzerland) or the Loop blood collection device available from Loop Medical (Lausanne, Switzerland).

In other aspects, the capillary blood sample is obtained or collected from a subject in a decentralized setting. For example, the capillary blood sample can be obtained or collected from an urgent care clinic, a pharmacy, a grocery or other convenience store, a residence, a workplace, and/or a government office.

In addition or alternatively, in still yet further aspects, the capillary blood sample is obtained from the subject by a user who is not trained in collecting blood (e.g., by someone other than a trained phlebotomist, a nurse, a medical assistant and/or physician). For example, the capillary blood sample can be obtained by the subject him or herself, a relative, friend, a co-worker, a coach, a pharmacist, and/or any other individual. In still yet further aspects, the capillary blood sample is obtained from a subject by a robot.

In some aspects, the capillary blood sample obtained from the subject is in an amount of less than about 4 mL. In some aspects, the capillary blood sample obtained from the subject is less than about 3 mL. In some aspects, the capillary blood sample obtained from the subject is less than about 2 mL. In some aspects, the amount of capillary blood sample obtained from the subject is less than about 3.9 mL, about 3.8 mL, about 3.7 mL, about 3.6 mL, about 3.5 mL, about 3.4 mL, about 3.3 mL, about 3.2 mL, about 3.1 mL, about 3.0 mL, about 2.9 mL, about 2.8 mL, about 2.7 mL, about 2.6 mL, about 2.5 mL, about 2.4 mL, about 2.3 mL, about 2.2 mL, about 2.1 mL, about 2.0 ml, about 1.9 mL, about 1.8 mL, about 1.7 mL, about 1.6 mL, about 1.5 mL, about 1.4 mL, about 1.3 mL, about 1.2 mL, about 1.1 mL, about 1.0 mL, about 0.9 mL, about 0.8 mL, about 0.7 mL, about 0.6 mL, or about 0.5 mL. In some aspects, higher volumes of capillary blood may be obtained when the sample collected is whole blood.

In some aspects, the capillary blood sample obtained from a subject is whole blood that is subjected to further or additional processing prior to determining the amount of UCH-L1, GFAP or UCH-L1 and GFAP in the sample. In some aspects, the sample is processed using centrifugation. In yet other aspects, the sample is processed using a plasma separation device which may comprise at least one filter, membrane and/or synthetic paper. For example, in some aspects, the plasma separation device that can be used is the apparatus described in Section 4. The plasma separation device can separate whole blood into serum and/or plasma which can then be used in the methods described herein.

In other aspects, the plasma separation device can be in fluid communication with or operably linked, coupled and/or removably coupled to a microsampling device as part of a system as described in further detail in Section 5. In other aspects, the plasma separation device can be integrated into the microsampling device as described in further detail in Section 5.

In still yet other aspects, the plasma separation device can be in fluid communication with or operably linked, coupled and/or removably coupled to an aperture of a reaction vessel as described in further detail in Section 5. For example, in some aspects, the reaction vessel is a cartridge such as those used in a point-of-care device.

In still other aspects, the plasma separation device can be in fluid communication with or operably linked, coupled and/or removably coupled to a transfer tube as described in further detail in Section 5. In these aspects, the transfer can be in fluid communication with or operably linked, coupled and/or removably coupled to a reaction vessel as described in further detail in Section 5. In still further aspects, the plasma separation device can be integrated into the transfer tube. In yet other aspects, the transfer tube includes a cap or a stopper.

In some aspects, the method can include obtaining a sample within about 24 hours of an actual or suspected injury to the subject and contacting the sample with an antibody for a biomarker of TBI, such as UCH-L1, GFAP, or a combination thereof, to allow formation of a complex of the antibody and the biomarker. More specifically, the sample can be contacted with (a) an anti-UCH-L1 antibody; (b) an anti-GFAP antibody; or (c) an anti-UCH-L1 antibody and an anti-UCH-L1 antibody. The method also includes detecting the resulting antibody-biomarker complex.

In some aspects, the sample is taken from the subject (e.g., human subject) within about 24 hours of injury of an actual or suspected injury to the head. For example, the sample can be taken from the subject (e.g., a human subject) within about 0 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 30 minutes, about 60 minutes, about 90 minutes, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, or within about 24 hours, after an actual or suspected injury to the head.

The amount of the UCH-L1, GFAP, or UCH-L1 and GFAP in the sample obtained from the subject can be determined using an analog assay, a digital assay, or a combination of an analog and a digital assay. In some aspects, the amount of the UCH-L1, GFAP, or UCH-L1 and GFAP is determined using an analog assay. In other aspects, the amount of the UCH-L1, GFAP, or UCH-L1 and GFAP can be determined using a digital assay. In some aspects, a digital assay is used when the ratio between capillary blood and venous blood amount of UCH-L1, GFAP, or UCH-L1 and GFAP is or is expected to be equal to or less than 1. In yet other aspects, an analog assay is used when the ratio between capillary blood and venous blood amount of UCH-L1, GFAP, or UCH-L1 and GFAP is or is expected to be greater than 1.

Examples of assays that can be used to determine the amount of UCH-L1, GFAP, or UCH-L1 and GFAP include an immunoassay, such as an enzyme immunoassay (EIA), an enzyme linked immunosorbent assay (ELISA), a fluorescent immunoassay, a chemiluminescence Immunoassay (CLIA), a radioimmunoassay (RIA), a microparticle enzyme immunoassay (MEIA), a turbidimetric immunoassay, etc. In yet other aspects, the assay may be a clinical chemistry assay such as, for example, a photometry, a spectrophotometry, an absorbence, a fluorescence, a turbidimetry, a nephelometry, a potentiometry and/or an electrophoresis assay. In yet further aspects, the assay may be a combination of an immunoassay and a clinical chemistry assay. In still other aspects, the assay may be a single molecule detection assay.

The at least one assay for GFAP and the at least one assay for UCH-L1 may be performed simultaneously. Alternatively, the assay for GFAP and the assay for UCH-L1 may be performed sequentially. The assays may be performed sequentially, in any order. For example, the assay for GFAP may be performed first, followed by the assay for UCH-L1. As another example, the assay for UCH-L1 may be performed first, followed by the assay for GFAP.

In some aspects, the at least one assay for GFAP and/or the at least one assay for UCH-L1 are each performed or capable of being performed in less than about 30 minutes. In yet some other aspects, the at least one assay for GFAP and/or the at least one assay for UCH-L1 are each are each performed or capable of being performed in less than about 25 minutes. In still further aspects, the at least one assay for GFAP and/or the at least one assay for UCH-L1 are each are each performed or capable of being performed in about less than about 20 minutes. In still yet other aspects, the at least one assay for GFAP and/or the at least one assay for UCH-L1 are each are each performed or capable of being performed in less than about 18 minutes. In still yet other aspects, the at least one assay for GFAP and/or the at least one assay for UCH-L1 are each are each performed or capable of being performed in about 4 to about 20 minutes. In still further aspects, the at least one assay for GFAP and/or the at least one assay for UCH-L1 are each are each performed or capable of being performed in about 15 to about 18 minutes.

In some aspects, the at least one assay for GFAP and/or at the at least one assay for UCH-L1 are each performed or capable of being performed in about 4 minutes. In some aspects, the at least one assay for GFAP and at the at least one assay for UCH-L1 are each performed or capable of being performed in about 5 minutes. In some aspects, the at least one assay for GFAP and/or at the at least one assay for UCH-L1 are each performed or capable of being performed in about 6 minutes. In some aspects, the at least one assay for GFAP and/or at the at least one assay for UCH-L1 are each performed or capable of being performed in about 7 minutes. In some aspects, the at least one assay for GFAP and/or at the at least one assay for UCH-L1 are each performed or capable of being performed in about 8 minutes. In some aspects, the at least one assay for GFAP and/or at the at least one assay for UCH-L1 are each performed or capable of being performed in about 9 minutes. In some aspects, the at least one assay for GFAP and/or at the at least one assay for UCH-L1 are each performed or capable of being performed in about 10 minutes. In some aspects, the at least one assay for GFAP and/or at the at least one assay for UCH-L1 are each performed or capable of being performed in about 11 minutes. In some aspects, the at least one assay for GFAP and/or at the at least one assay for UCH-L1 are each performed or capable of being performed in about 12 minutes. In some aspects, the at least one assay for GFAP and/or at the at least one assay for UCH-L1 are each performed or capable of being performed in about 13 minutes. In some aspects, the at least one assay for GFAP and/or at the at least one assay for UCH-L1 are each performed or capable of being performed in about 14 minutes. In some aspects, the at least one assay for GFAP and/or at the at least one assay for UCH-L1 are each performed or capable of being performed in about 15 minutes. In some aspects, the at least one assay for GFAP and/or at the at least one assay for UCH-L1 are each performed or capable of being performed in about 16 minutes. In some aspects, the at least one assay for GFAP and/or at the at least one assay for UCH-L1 are each performed or capable of being performed in about 17 minutes. In some aspects, the at least one assay for GFAP and/or at the at least one assay for UCH-L1 are each performed or capable of being performed in about 18 minutes. In some aspects, the at least one assay for GFAP and/or at the at least one assay for UCH-L1 are each performed or capable of being performed in about 19 minutes. In some aspects, the at least one assay for GFAP and/or at the at least one assay for UCH-L1 are each performed or capable of being performed in about 20 minutes.

In some aspects, the subject has received a Glasgow Coma Scale score before or after the assay is performed. In some aspects, the subject (e.g., a human subject) is suspected as having moderate, severe, or moderate to severe traumatic brain injury based on the Glasgow Coma Scale score. In some aspects, the reference level of the biomarker, such as UCH-L1, GFAP, or a combination thereof, is correlated with subjects having moderate, severe, or moderate to severe traumatic brain injury. In some aspects, the reference level of the biomarker, such as UCH-L1, GFAP, or a combination thereof, is correlated with a Glasgow Coma Scale score of 9-13 (a moderate TBI). In some aspects, the reference level of the biomarker, such as UCH-L1, GFAP, or a combination thereof, is correlated with a Glasgow Coma Scale score of 3-8 (a severe TBI). In some aspects, the reference level of the biomarker, such as UCH-L1, GFAP, or a combination thereof, is correlated with a Glasgow Coma Scale score of 3-12 (a moderate, severe, or moderate to severe TBI). In some aspects, the subject is suspected as having mild traumatic brain injury based on the Glasgow Coma Scale score. In some aspects, the reference level of the biomarker, such as UCH-L1, GFAP, or a combination thereof, is correlated with subjects having mild traumatic brain injury. In some aspects, the reference level of the biomarker, such as UCH-L1, GFAP, or a combination thereof, is correlated with a Glasgow Coma Scale score of 13-15 (mild TBI).

Generally, a reference level of the biomarker, such as UCH-L1, GFAP, or a combination thereof, can also be employed as a benchmark against which to assess results obtained upon assaying a test sample for the biomarker, such as UCH-L1, GFAP, or a combination thereof. Generally, in making such a comparison, the reference level of the biomarker, such as UCH-L1, GFAP, or a combination thereof, is obtained by running or conducting a particular assay a sufficient number of times and under appropriate conditions such that a linkage or association of analyte presence, amount or concentration with a particular stage or endpoint of TBI or with particular indicia can be made. Typically, the reference level of the biomarker, such as UCH-L1, GFAP, or a combination thereof, is obtained with assays of reference subjects (or populations of subjects). The biomarker, such as UCH-L1, GFAP, or a combination thereof, measured can include fragments thereof, degradation products thereof, and/or enzymatic cleavage products thereof.

In certain aspects, the reference level may be correlated with control subjects (e.g., human subjects) that have not sustained a head injury.

In some aspects, the reference level for UCH-L1 is from about 320 to about 400 μg/mL. In other aspects, the reference level for UCH-L1 is about 360 μg/mL. In still further aspects, the reference level for UCH-L1 is about 400 μg/mL.

In some aspects, the reference level for UCH-L1 is from about 320 to about 400 μg/mL and the sample is obtained from the subject within about 24 hours or less. In other aspects, the reference level for UCH-L1 is about 360 μg/mL and the sample is obtained from the subject within about 24 hours or less. In yet other aspects, the reference level for UCH-L1 is about 400 μg/mL and the sample is obtained from the subject within about 24 hours or less.

In some aspects, the reference level for GFAP is from about 25 to about 40 μg/mL. In other aspects, the reference level for GFAP is about 30 μg/mL. In still further aspects, the reference level for GFAP is about 35 μg/mL.

In some aspects, the reference level for GFAP is from about 25 to about 40 μg/mL and the sample is obtained from the subject within about 24 hours or less. In other aspects, the reference level for GFAP is about 30 μg/mL and the sample is obtained from the subject within about 24 hours or less. In yet other aspects, the reference level for GFAP is about 35 μg/mL and the sample is obtained from the subject within about 24 hours or less.

In some aspects, the reference level for UCH-L1 is about 360 μg/mL and the reference level for GFAP is about 30 μg/mL. In yet other aspects, the reference level for UCH-L1 is about 400 μg/mL and the reference level for GFAP is about 35 μg/mL. In still further aspects, the reference level for UCH-L1 is about 360 μg/mL and the reference level for GFAP is about 30 μg/mL and the sample is obtained from the subject within about 24 hours or less. In yet other aspects, the reference level for UCH-L1 is about 400 μg/mL and the reference level for GFAP is about 35 μg/mL and the sample is obtained from the subject within about 24 hours or less.

In some aspects, the method comprises performing at least one assay for UCH-L1 and at least one assay for GFAP in at least one sample obtained from the subject, and determining whether the amount (e.g., level) of GFAP and UCH-L1 in the subject is elevated based upon the results of the assays. In some aspects, the method comprises determining that the amount of GFAP and UCH-L1 in the subject is elevated. In some aspects, the method comprises determining that the amount of GFAP and UCH-L1 in the subject is elevated when the amount of GFAP in the sample is equal to or above 30 μg/mL and the level of UCH-L1 is below about 360 μg/mL, cannot be determined by the assay for UCH-L1, or is not reported by the assay for UCH-L1. In some aspects, the method comprises determining that the amount of GFAP and UCH-L1 in a sample obtained from a subject is elevated when the level of GFAP is equal to or above about 30 μg/mL and level of UCH-L1 is equal to or above about 360 μg/mL. In some aspects, the method comprises determining that the amount of GFAP and UCH-L1 in the subject is elevated when the level of GFAP cannot be determined by the assay for GFAP or is not reported by the assay for GFAP, and the level of UCH-L1 is equal to or above about 360 μg/mL.

In some aspects, the method comprises determining that the amount of GFAP and UCH-L1 in the subject not elevated. In some aspects, the method comprises determining that the amount of GFAP and UCH-L1 in the subject is not elevated when the level of GFAP in the sample is below about 30 μg/mL and level of UCH-L1 in the sample is below about 360 μg/mL.

In some aspects, the method comprises determining that the assays for UCH-L1 and GFAP should be repeated. In some aspects, the method comprises determining that the assays for UCH-L1 and GFAP should be repeated when the amount of GFAP is below about 30 μg/mL and the level of UCH-L1 cannot be determined by the assay for UCH-L1 or is not reported by the assay for UCH-L1. In some aspects, the method comprises determining that the assays for UCH-L1 and GFAP should be repeated when the amount of GFAP cannot be determined by the assay or is not reported by the assay for GFAP and the level of UCH-L1 is below about 360 μg/mL. In some aspects, the method comprises determining that the assays for UCH-L1 and GFAP should be repeated when the amount of GFAP cannot be determined by the assay for GFAP or is not reported by the assay for GFAP and the level of UCH-L1 cannot be determined by the UCH-L1 or is not reported by the assay for UCH-L1.

In some aspects, the method comprises performing at least one assay for UCH-L1 and at least one assay for GFAP in at least one sample obtained from the subject, and determining whether the amount of GFAP and UCH-L1 in the subject is elevated based upon the results of the assays. In some aspects, the method comprises determining that the amount of GFAP and UCH-L1 in the subject is elevated. In some aspects, the method comprises determining that the amount of GFAP and UCH-L1 in the subject is elevated when the amount of GFAP in the sample is equal to or above 35 μg/mL and the amount of UCH-L1 is below about 400 μg/mL, cannot be determined by the assay for UCH-L1, or is not reported by the assay for UCH-L1. In some aspects, the method comprises determining that the amount of GFAP and UCH-L1 in the subject is elevated when the amount of GFAP is equal to or above about 35 μg/mL and level of UCH-L1 is equal to or above about 400 μg/mL. In some aspects, the method comprises determining that the amount GFAP and UCH-L1 in the subject is elevated when the amount of GFAP cannot be determined by the assay for GFAP or is not reported by the assay for GFAP, and the amount of UCH-L1 is equal to or above about 400 μg/mL.

In some aspects, the method comprises determining that the amount of GFAP and UCH-L1 in the subject is not elevated. In some aspects, the method comprises determining that the amount of GFAP and UCH-L1 in the subject is not elevated when the amount of GFAP in the sample is below about 35 μg/mL and amount of UCH-L1 in the sample is below about 400 μg/mL.

In some aspects, the method comprises determining that the assays for UCH-L1 and GFAP should be repeated. In some aspects, the method comprises determining that the assays for UCH-L1 and GFAP should be repeated when the amount of GFAP is below about 35 μg/mL and the amount of UCH-L1 cannot be determined by the assay for UCH-L1 or is not reported by the assay for UCH-L1. In some aspects, the method comprises determining that the assays for UCH-L1 and GFAP should be repeated when the amount of GFAP cannot be determined by the assay for GFAP or is not reported by the assay for GFAP and the amount of UCH-L1 is below about 400 μg/mL. In some aspects, the method comprises determining that the assays for UCH-L1 and GFAP should be repeated when the amount of GFAP cannot be determined by the assay for GFAP or is not reported by the assay for GFAP and the amount of UCH-L1 cannot be determined by the UCH-L1 or is not reported by the assay for UCH-L1.

In some aspects, the method further comprises performing a head computed tomography (CT) scan, a magnetic resonance imaging (MRI) procedure, or both a CT scan or a MRI procedure on the subject when the subject's levels of GFAP and UCH-L1 are elevated. For example, in some aspects the method further comprises performing a head CT scan on the subject when the subject's levels of GFAP and UCH-L1 are elevated. As another example, in some aspects the method further comprises performing an MRI procedure on the subject when the subject's levels of GFAP and UCH-L1 are elevated. In some aspects, the method further comprises performing a head CT scan and an MRI procedure on the subject when the subject's levels of GFAP and UCH-L1 are elevated.

In yet further aspects, after the amount of UCH-L1 and/or GFAP in the capillary blood sample is determined using the methods described herein, a result is obtained. In some further aspects, the result can be further processed. Specifically, this further processing involves selecting a conversion factor for comparing the amount of UCH-L1 and/or GFAP in the capillary whole blood or plasma sample with the amount of UCH-L1 and/or GFAP in venous whole blood or plasma.

In some aspects, when the assay is for UCH-L1, the conversion factor can be from about 2.5:1.0 (capillary whole blood or plasma to venous whole blood or plasma) to about 1.5:1.0 ((capillary whole blood or plasma to venous whole blood or plasma)). While not wishing to be bound by any theory, it is believed that the higher levels of UCH-L1 demonstrated in capillary blood samples may be the result of a noise level, and not due, for example, to a subject suffering a traumatic brain injury.

In other aspects, when the assay is for GFAP, the conversion factor can be about 1.0:1.0 (capillary whole blood or plasma to venous whole blood or plasma).

Once the conversion factor is selected, the processing further involves normalizing the amount of UCH-L1 and/or GFAP in the capillary whole blood or plasma sample with the amount of UCH-L1 and/or GFAP from venous whole blood or plasma by applying the conversion factor to the amount of UCH-L1 and/or GFAP in the sample. For example, the amount of the UCH-L1 and/or GFAP in the capillary whole blood or plasma sample can be multiplied by the conversion factor to provide the normalized amount of the UCH-L1 and/or GFAP in the sample.

In some aspects, the processing of the amount of UCH-L1 and/or GFAP (e.g., the result) can be by a processing system which comprises a computer processor and a non-transitory computer memory comprising one or more computer programs, in conjunction with said computer process which are configured to select a conversion factor for comparing the amount of UCH-L1 and/or GFAP in the capillary whole blood or plasma sample with the amount of UCH-L1 and/or GFAP in venous whole blood or plasma and normalize the amount of UCH-L1 and/or GFAP in the capillary whole blood or plasma sample with the amount of UCH-L1 and/or GFAP from venous whole blood or plasma by applying the selected conversion factor to the amount UCH-L1 and/or GFAP in the sample.

This result or processed result can be communicated (e.g., reported) for further analysis, interpretation, processing and/or display. The result can be communicated (e.g., reported) by a computer, in a document and/or spreadsheet, on a mobile device (e.g., a smart phone), on a website, in an e-mail, or any combination thereof. In some aspects, the result can displayed not as a number, but as a visual signal (e.g., a line or bar) that can be read/interpreted by a reader or the naked eye.

In some aspects, the result of UCH-L1, GFAP, or UCH-L1 and GFAP determined in the sample is communicated by being displayed, such as on an instrument. In further aspects, the result is displayed as indicating that the amount of UCH-L1 and/or GFAP in a subject is elevated, are not elevated, or that the assay(s) for UCH-L1 and/or GFAP should be repeated.

Suitable instruments for use in the methods described herein include a higher throughput assay analyzer (e.g., the ARCHITECT platform marketed by Abbott Laboratories) or a point-of-care device (e.g., i-STAT and i-STAT Alinity devices marketed by Abbott Laboratories) that may contain a user interface that can display the determination. Other instruments for use in the methods described herein include a lateral flow device (e.g., a lateral flow device with a reader).

In some aspects, the result of UCH-L1, GFAP, or UCH-L1 and GFAP is communicated or capable of being communicated in about 4 minutes to about 40 minutes from the time the sample is collected (such as, for example, from the time of injury or suspected injury). In other aspects, the result is communicated or capable of being communicated in about 4 minutes to about 30 minutes from the time the sample is collected (e.g., the time of injury or suspected injury). In yet other aspects, the result is communicated or capable of being communicated in about 4 minutes to about 20 minutes from the time the sample is collected (such as, for example, from the time of injury or suspected injury). In some aspects, the result is communicated or capable of being communicated in about 40 minutes or less, about 39 minutes or less, about 38 minutes or less, about 37 minutes or less, about 36 minutes, or less about 35 minutes, less about 34 minutes or less, about 33 minutes or less, about 32 minutes or less, about 31 minutes or less, about 30 minutes or less, about 29 minutes or less, about 28 minutes or less, about 27 minutes or less, about 26 minutes or less, about 25 minutes or less, about 24 minutes or less, about 23 minutes or less, about 22 minutes or less, about 21 minutes or less, about 20 minutes, about 19 minutes, about 18 minutes, about 17 minutes, about 16 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes, or about 4 minutes from the time the sample is collected (such as, for example, from the time of injury or suspected injury).

In some aspects in the above method, the time from collection of the sample (from the subject) until the time a result for GFAP, UCH-L1 or GFAP and UCH-L1 is communicated is less than about 30 minutes. In yet other aspects in the above method, the time from collection of the sample until the time a result for GFAP, UCH-L1 or GFAP and UCH-L1 is communicated is less than about 25 minutes. In yet some other aspects in the above method, the time from collection of the sample until the time a result for GFAP, UCH-L1 or GFAP and UCH-L1 is communicated is less than about 20 minutes. In yet still further aspects in the above method, the time from collection of the sample until the time a result for GFAP, UCH-L1 or GFAP and UCH-L1 is communicated is less than about 18 minutes. In some aspects in the above method, the time from collection of the sample until the time a result for GFAP, UCH-L1 or GFAP and UCH-L1 is communicated is less than about 15 minutes. In some aspects in the above method, the time from collection of the sample until the time a result for GFAP, UCH-L1 or GFAP and UCH-L1 is communicated is about 4 to about 20 minutes. In some aspects in the above method, the time from collection of the sample until the time a result for GFAP, UCH-L1 or GFAP and UCH-L1 is communicated is about 15 minutes to about 20 minutes.

In some aspects, the instrument contains software to execute one or more tasks, including the performance of the methods and algorithms described herein. In some aspects, the instrument contains software to automatically determine the next appropriate step in a methods and algorithms as described herein. For example, the instrument may contain software that determines the amount or presence of an analyte of interest. The software may display this determination, such as on a graphical user interface.

In some aspects, the instrument stores software that instructs a processor to execute a given task. In some aspects, the software stores machine readable instructions that instruct a processor to execute a given task. The machine-readable instructions may be one or more executable programs or portion(s) of an executable program for execution by a computer. The programs may be embodied in software stored on a non-transitory computer readable storage medium such as a CD-ROM, a floppy disk, a hard drive, a DVD, a Blu-ray disk, or a memory associated with the processors. Alternatively, the entire programs and/or parts thereof could alternatively be executed by a device other than the processors and/or embodied in firmware or dedicated hardware. Additionally or alternatively, processes may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine-readable instructions may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine-readable instructions may be fragmented and stored on one or more storage devices and/or computing devices (e.g., servers). The machine-readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine-readable instructions may be stored in multiple parts, which are individually compressed, encrypted, and stored on separate computing devices, wherein the parts when decrypted, decompressed, and combined form a set of executable instructions that implement a program such as that described herein.

In another example, the machine-readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine-readable instructions may need to be configured (e.g., settings stored, data input, network addresses recorded, etc.) before the machine-readable instructions and/or the corresponding program(s) can be executed in whole or in part. Thus, the disclosed machine-readable instructions and/or corresponding program(s) are intended to encompass such machine-readable instructions and/or program(s) regardless of the particular format or state of the machine-readable instructions and/or program(s) when stored or otherwise at rest or in transit.

The machine-readable instructions described herein can be represented by any past, present, or future instruction language, scripting language, programming language, etc. For example, the machine-readable instructions may be represented using any of the following languages: C, C++, Java, C#, Perl, Python, JavaScript, HyperText Markup Language (HTML), Structured Query Language (SQL), Swift, etc.

The machine readable instructions may be stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

3. Methods for Determining an Amount or Presence of CK-MB, β-hCG, TSH, Homocysteine, Free T4, or any Combinations Thereof in a Capillary Blood Sample Obtained from a Subject

In one embodiment, the present disclosure relates to improved methods for determining an amount (e.g., a quantitative measure) or the presence (e.g., a qualitative measure) of CK-MB, β-hCG, TSH, homocysteine, free T4, or any combinations thereof in a capillary blood sample obtained from a subject (e.g., a human subject). In some aspects, the amount or presence of CK-MB determined in the capillary blood sample obtained according to the methods described herein can be used to diagnose myocardial infarction. In some aspects, a subject is determined as having a myocardial infarction when the amount of CK-MB in the sample is higher than a reference level of a biomarker (e.g., CK-MB).

In still other aspects, the amount or presence of β-hCG determined in the capillary blood sample obtained according to the methods described herein can be used to determine if a subject is pregnant. In some aspects, a subject (e.g., a female subject) is determined to likely be pregnant if the amount of β-hCG in the sample is higher than a reference level of a biomarker (e.g., β-hCG).

In still other aspects, the amount or presence of TSH determined in the capillary blood sample obtained according to the methods described herein can be used to diagnose thyroid disease in a subject, treat thyroid disease in a subject, or in any combination thereof. In some aspects, a subject is determined as having a thyroid disease when the amount of TSH in the sample is higher than a reference level of a biomarker (e.g., TSH).

In still other aspects, the amount or presence of homocysteine determined in the capillary blood sample obtained according to the methods described herein can be used to diagnose hyperhomocysteinemia, homocystinuria, or hyperhomocysteinemia and homocystinuria in a subject, or treat subjects having hyperhomocysteinemia, homocystinuria, or hyperhomocysteinemia and homocystinuria, or in any combination thereof. In some aspects, a subject is determined as having a hyperhomocysteinemia, homocystinuria, or hyperhomocysteinemia and homocystinuria when the amount of homocysteine in the sample is higher than a reference level of a biomarker (e.g., homocysteine).

In still other aspects, the amount or presence of free T4 determined in the capillary blood sample obtained according to the methods described herein can be used to diagnose thyroid disease in a subject, treat thyroid disease in a subject, or in any combination thereof. In some aspects, a subject is determined as having a thyroid disease when the amount of free T4 in the sample is higher than a reference level of a biomarker (e.g., free T4).

In some aspects, the sample obtained from a subject (e.g., a human subject) is a capillary blood sample, such as, whole blood, serum or plasma. In some aspects, the capillary blood sample is whole blood. In other aspects, the capillary blood sample is serum. In yet other aspects, the capillary blood sample is plasma.

The capillary blood sample can be obtained by extracting the sample through the skin (e.g., such as the fingers and/or toes, a hand, a foot (including the heel), an earlobe, a location on the arms and/or legs, chest, back, head, or any combinations thereof) of a subject. In some aspects, the capillary blood sample is extracted from the fingers or toes. In other aspects, the whole capillary blood sample is extracted from the arms or legs. In still other aspects, the capillary blood sample is obtained from the hands or feet. In still other aspects, the capillary blood sample is obtained from the chest or back. In yet other aspects, the capillary blood sample is obtained from an earlobe. In still other aspects, the capillary blood sample is obtained from the head.

In some aspects, the capillary blood sample obtained from the subject is obtained without the use of a syringe, needle (e.g., 21-gauge needle, a butterfly needle, etc.), or any other suitable device, or any combination thereof which are typically used to draw blood (e.g., venous blood). Instead, the capillary blood sample is obtained using a self-or other-administered blood collection device. Examples of self-or other-administered blood collection devices include microsampling devices. Example microsampling devices which can be used herein include the TAP device available from YourBio Health, Inc. (Cambridge, MA) as well as the device described in U.S. Pat. No. 9,113,836, the contents of which are herein incorporated by reference, the Tasso+, Tasso-M20, and Tasso-ST devices available from Tasso, Inc. (Seattle, WA), the One Draw device available from Draw Bridge Health (San Diego, CA), PBS-1000 from PreciHealth (Neuchatel, Switzerland) or the Loop blood collection device available from Loop Medical (Lausanne, Switzerland).

In other aspects, the capillary blood sample is obtained or collected from a subject in a decentralized setting. For example, the capillary blood sample can be obtained or collected from an urgent care clinic, a pharmacy, a grocery or other convenience store, a residence, a workplace, and/or a government office.

In addition or alternatively, in still yet further aspects, the capillary blood sample is obtained from the subject by a user who is not trained in collecting blood (e.g., by someone other than a trained phlebotomist, a nurse, a medical assistant and/or physician). For example, the capillary blood sample can be obtained by the subject him or herself, a relative, friend, a co-worker, a coach, a pharmacist, and/or any other individual. In still yet further aspects, the capillary blood sample is obtained from a subject by a robot.

In some aspects, the capillary blood sample obtained from the subject is in an amount of less than about 4 mL. In some aspects, the capillary blood sample obtained from the subject is less than about 3 mL. In some aspects, the capillary blood sample obtained from the subject is less than about 2 mL. In some aspects, the amount of capillary blood sample obtained from the subject is less than about 3.9 mL, about 3.8 mL, about 3.7 mL, about 3.6 mL, about 3.5 mL, about 3.4 mL, about 3.3 mL, about 3.2 mL, about 3.1 mL, about 3.0 mL, about 2.9 mL, about 2.8 mL, about 2.7 mL, about 2.6 mL, about 2.5 mL, about 2.4 mL, about 2.3 mL, about 2.2 mL, about 2.1 mL, about 2.0 ml, about 1.9 mL, about 1.8 mL, about 1.7 mL, about 1.6 mL, about 1.5 mL, about 1.4 mL, about 1.3 mL, about 1.2 mL, about 1.1 mL, about 1.0 mL, about 0.9 mL, about 0.8 mL, about 0.7 mL, about 0.6 mL, or about 0.5 mL. In some aspects, higher volumes of capillary blood may be obtained when the sample collected is whole blood.

In some aspects, the capillary blood sample obtained from a subject is whole blood that is subjected to further or additional processing prior to determining the amount of CK-MB, β-hCG or CK-MB and β-hCG in the sample. In some aspects, the sample is processed using centrifugation. In yet other aspects, the sample is processed using a plasma separation device which may comprise at least one filter, membrane and/or synthetic paper. For example, in some aspects, the plasma separation device that can be used is the apparatus described in Section 4. The plasma separation device can separate whole blood into serum and/or plasma which can then be used in the methods described herein.

In other aspects, the plasma separation device can be in fluid communication with or operably linked, coupled and/or removably coupled to a microsampling device as part of a system as described in further detail in Section 5. In other aspects, the plasma separation device can be integrated into the microsampling device as described in further detail in Section 5.

In still yet other aspects, the plasma separation device can be in fluid communication with or operably linked, coupled and/or removably coupled to an aperture of a reaction vessel as described in further detail in Section 5. For example, in some aspects, the reaction vessel is a cartridge such as those used in a point-of-care device.

In still other aspects, the plasma separation device can be in fluid communication with or operably linked, coupled and/or removably coupled to a transfer tube as described in further detail in Section 5. In these aspects, the transfer can be in fluid communication with or operably linked, coupled and/or removably coupled to a reaction vessel as described in further detail in Section 5. In still further aspects, the plasma separation device can be integrated into the transfer tube. In yet other aspects, the transfer tube includes a cap or a stopper.

In some aspects, with respect to the biomarker CK-MB, the method can include obtaining a sample within about 24 hours of an actual or suspected of having or having had a myocardial infarction and contacting the sample with an antibody for CK-MB to allow formation of a complex of the antibody and the biomarker. More specifically, the sample can be contacted with an anti-CK-MB antibody. The method also includes detecting the resulting antibody-biomarker complex.

In some aspects, the sample is taken from the subject (e.g., human subject) within about 24 hours of injury of an actual or suspected myocardial infarction. For example, the sample can be taken from the subject (e.g., a human subject) within about 0 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 30 minutes, about 60 minutes, about 90 minutes, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, or within about 24 hours, after an actual or suspected myocardial infarction.

In other aspects, with respect to the biomarker β-hCG, the method can include obtaining a sample from a subject, such as, for example, a subject that is suspected of being pregnant, and contacting the sample with an antibody for β-hCG to allow formation of a complex of the antibody and the biomarker. More specifically, the sample can be contacted with an anti-β-hCG antibody. The method also includes detecting the resulting antibody-biomarker complex.

In yet other aspects, with respect to the biomarker TSH and/or T4, the method can include obtaining a sample from a subject suspected of or suffering from thyroid dysfunction or thyroid disease or being treated for thyroid disease, or any combinations thereof.

In still yet other aspects, with respect to the biomarker homocysteine, the method can include obtaining a sample from a subject suspected of or for suffering from diagnose hyperhomocysteinemia, homocystinuria, or hyperhomocysteinemia and homocystinuria or being treated for hyperhomocysteinemia, homocystinuria, or hyperhomocysteinemia and homocystinuria.

The amount of the CK-MB, β-hCG, TSH, homocysteine, free T4, or any combinations thereof, in the sample obtained from the subject can be determined using an analog assay, a digital assay, or a combination of an analog and a digital assay. In some aspects, the amount of the CK-MB, β-hCG, TSH, homocysteine, free T4, or any combinations thereof, is determined using an analog assay. In other aspects, the amount of the CK-MB, β-hCG, TSH, homocysteine, free T4, or any combinations thereof, can be determined using a digital assay. In some aspects, a digital assay is used when the ratio between capillary whole blood or plasma and venous whole blood or plasma amount of CK-MB, β-hCG, TSH, homocysteine, free T4, or any combinations thereof, is or is expected to be equal to or less than 1. In yet other aspects, an analog assay is used when the ratio between capillary blood and venous blood amount of CK-MB, β-hCG, TSH, homocysteine, free T4, or any combinations thereof, is or is expected to be greater than 1.

Examples of assays that can be used to determine the amount of CK-MB, β-hCG, TSH, homocysteine, free T4, or any combinations thereof, include an immunoassay, such as an enzyme immunoassay (EIA), an enzyme linked immunosorbent assay (ELISA), a fluorescent immunoassay, a chemiluminescence Immunoassay (CLIA), a radioimmunoassay (RIA), a microparticle enzyme immunoassay (MEIA), a turbidimetric immunoassay, etc. In yet other aspects, the assay may be a clinical chemistry assay such as, for example, a photometry, a spectrophotometry, an absorbance, a fluorescence, a turbidimetry, a nephelometry, a potentiometry and/or an electrophoresis assay. In yet further aspects, the assay may be a combination of an immunoassay and a clinical chemistry assay. In still other aspects, the assay may be a single molecule detection assay.

The at least one assay for CK-MB, the assay for β-hCG, the assay for TSH, the assay for homocysteine, and/or the assay free T4 may be performed simultaneously. Alternatively, the assay for the assay for β-hCG, the assay for TSH, the assay for homocysteine, and/or the assay free T4 hCG may be performed sequentially. The assays may be performed sequentially, in any order.

In some aspects, the at least one assay for CK-MB, at least one assay for β-hCG, the assay for TSH, the assay for homocysteine, and/or the assay free T4 are each performed or capable of being performed in less than about 30 minutes. In yet some other aspects, the at least one assay for CK-MB, at least one assay for-hCG, the assay for TSH, the assay for homocysteine, and/or the assay free T4 are each performed or capable of being performed in less than about 25 minutes. In some aspects, the at least one assay for CK-MB, the at least one assay for β-hCG, the assay for TSH, the assay for homocysteine, and/or the assay free T4 are each performed or capable of being performed in about less than about 20 minutes. In some aspects, the at least one assay for CK-MB, the at least one assay for β-hCG, the assay for TSH, the assay for homocysteine, and/or the assay free T4 are each performed or capable of being performed in less than about 18 minutes. In still yet other aspects, the at least one assay for CK-MB, at least one assay for β-hCG, the assay for TSH, the assay for homocysteine, and/or the assay free T4 are each performed or capable of being performed in about 4 to about 20 minutes. In some aspects, the at least one assay for CK-MB, the at least one assay for β-hCG, the assay for TSH, the assay for homocysteine, and/or the assay free T4 are each performed or capable of being performed in about 15 to about 18 minutes.

In some aspects, the at least one assay for CK-MB, the at least one assay for β-hCG, the assay for TSH, the assay for homocysteine, and/or the assay free T4 are each performed or capable of being performed in about 4 minutes. In some aspects, the at least one assay for CK-MB, the at least one assay for β-hCG, the assay for TSH, the assay for homocysteine, and/or the assay free T4 are each performed or capable of being performed in about 5 minutes. In some aspects, the at least one assay for CK-MB, the at least one assay for β-hCG, the assay for TSH, the assay for homocysteine, and/or the assay free T4 are each performed or capable of being performed in about 6 minutes. In some aspects, the at least one assay for CK-MB, the at least one assay for β-hCG, the assay for TSH, the assay for homocysteine, and/or the assay free T4 are each performed or capable of being performed in about 7 minutes. In some aspects, the at least one assay for CK-MB, the at least one assay for β-hCG, the assay for TSH, the assay for homocysteine, and/or the assay free T4 are each performed or capable of being performed in about 8 minutes. In some aspects, the at least one assay for CK-MB, the at least one assay for β-hCG, the assay for TSH, the assay for homocysteine, and/or the assay free T4 are each performed or capable of being performed in about 9 minutes. In some aspects, the at least one assay for CK-MB, the at least one assay for β-hCG, the assay for TSH, the assay for homocysteine, and/or the assay free T4 are each performed or capable of being performed in about 10 minutes. In some aspects, the at least one assay for CK-MB, the at least one assay for β-hCG, the assay for TSH, the assay for homocysteine, and/or the assay free T4, are each performed or capable of being performed in about 11 minutes. In some aspects, the at least one assay for CK-MB, the at least one assay for β-hCG, the assay for TSH, the assay for homocysteine, and/or the assay free T4 are each performed or capable of being performed in about 12 minutes. In some aspects, the at least one assay for CK-MB, the at least one assay for β-hCG, the assay for TSH, the assay for homocysteine, and/or the assay free T4, the assay for TSH, the assay for homocysteine, and/or the assay free T4 are each performed or capable of being performed in about 13 minutes. In some aspects, the at least one assay for CK-MB, the at least one assay for β-hCG, the assay for TSH, the assay for homocysteine, and/or the assay free T4, are each performed or capable of being performed in about 14 minutes. In some aspects, the at least one assay for CK-MB, the at least one assay for-hCG, the assay for TSH, the assay for homocysteine, and/or the assay free T4, are each performed or capable of being performed in about 15 minutes. In some aspects, the at least one assay for CK-MB, the at least one assay for β-hCG, the assay for TSH, the assay for homocysteine, and/or the assay free T4, are each performed or capable of being performed in about 16 minutes. In some aspects, the at least one assay for CK-MB, the at least one assay for β-hCG, the assay for TSH, the assay for homocysteine, and/or the assay free T4, are each performed or capable of being performed in about 17 minutes. In some aspects, the at least one assay for CK-MB, the at least one assay for β-hCG, the assay for TSH, the assay for homocysteine, and/or the assay free T4, are each performed or capable of being performed in about 18 minutes. the at least one assay for CK-MB, the at least one assay for β-hCG, the assay for TSH, the assay for homocysteine, and/or the assay free T4, are each performed or capable of being performed in about 19 minutes. In some aspects, the at least one assay for CK-MB, the at least one assay for β-hCG, the assay for TSH, the assay for homocysteine, and/or the assay free T4, are each performed or capable of being performed in about 20 minutes.

Generally, a reference level of the biomarker, such as CK-MB, β-hCG, TSH, homocysteine, or free T4 or any combination thereof, can also be employed as a benchmark against which to assess results obtained upon assaying a test sample for the biomarker, such as CK-MB, β-hCG TSH, homocysteine, or free T4 or any combinations thereof. Generally, in making such a comparison, the reference level of the biomarker, such as CK-MB, β-hCG, TSH, homocysteine, or free T4 or any combination thereof, is obtained by running or conducting a particular assay a sufficient number of times and under appropriate conditions such that a linkage or association of analyte presence, amount or concentration with a particular stage or endpoint of an Illinois, disease and/or condition or with particular indicia can be made. Typically, the reference level of the biomarker, such as CK-MB, β-hCG, TSH, homocysteine, or free T4 or any combinations thereof, is obtained with assays of reference subjects (or populations of subjects). The biomarker, such as CK-MB, β-hCG, TSH, homocysteine, or free T4 or any combinations thereof, measured can include fragments thereof, degradation products thereof, and/or enzymatic cleavage products thereof.

In certain aspects, the reference level may be correlated with control subjects (e.g., human subjects) that have not had a myocardial infarction (e.g., in the case of CK-MB), or are not pregnant (e.g., in the case of β-hCG), do not suffer from thyroid dysfunction or thyroid disease (e.g., in the case of TSH and/or free T4) or do not suffer from hyperhomocysteinemia, homocystinuria, or hyperhomocysteinemia and homocystinuria (e.g., in the case of homocysteine).

In yet further aspects, once the amount of CK-MB, β-hCG, TSH, homocysteine, or free T4 or any combinations thereof, is determined using the methods described herein, a result is obtained. This result can be further processed. Specifically, this further processing involves selecting a conversion factor for comparing the amount of CK-MB, β-hCG, TSH, homocysteine, and/or free T4 in the capillary whole blood or plasma sample with the amount of the same analyte in venous whole blood or plasma.

In some aspects, when the when the assay is for CK-MB, the conversion factor can be from about 0.5:1.0 (capillary whole blood or plasma to venous whole blood or plasma). to about 1:0:1.2 (capillary whole blood or plasma to venous whole blood or plasma). In other aspects, the conversion factor is about 0.8:1.0 (capillary whole blood or plasma to venous whole blood or plasma). In yet further aspects, when the assay is for CK-MB, the conversion factor can be from about 1.2:1.0 (venous whole blood or plasma to capillary whole blood or plasma) to about 1:0.5 (venous whole blood or plasma to capillary whole blood or plasma). In other aspects, the conversion factor is about 1:0.8 (venous whole blood or plasma to capillary whole blood or plasma).

In other aspects, when the assay is for β-hCG, the conversion factor can be 0.8:1.0 (capillary whole blood or plasma to venous whole blood or plasma). to about 1.0:1.4 (capillary whole blood or plasma to venous whole blood or plasma). In other aspects, the conversion factor is about 1:1 (capillary whole blood or plasma to venous whole blood or plasma). In yet other aspects, for the analyte to be detected is the β-hCG, the conversion factor can be 1.4:1.0 (venous whole blood or plasma to capillary whole blood or plasma) to about 1:0.8 (venous whole blood or plasma to capillary whole blood or plasma). In other aspects, the conversion factor is about 1:1 (venous whole blood or plasma to capillary whole blood or plasma).

In other aspects, when the assay is for TSH, the conversion factor can be about 0.60:1.0 (capillary whole blood or plasma to venous whole blood or plasma) to about 1.5:1.0 (capillary whole blood or plasma to venous whole blood or plasma). In other aspects, the conversion factor is about 0.75:1.0 (capillary whole blood or plasma to venous whole blood or plasma) to about 1.2:1.0 (capillary whole blood or plasma to venous whole blood or plasma).

In still other aspects, when the assay is for homocysteine, the conversion factor can be 1.5:1.0 (capillary whole blood or plasma to venous whole blood or plasma) to about 0.60:1.0 (capillary whole blood or plasma to venous whole blood or plasma). In other aspects, the conversion factor is about 1.2:1.0 (capillary whole blood or plasma to venous whole blood or plasma) to about 0.9:1.0 (capillary whole blood or plasma to venous whole blood or plasma)

In still other aspects, when the assay is for free T4, the conversion factor can be 0.60:1.0 (capillary whole blood or plasma to venous whole blood or plasma) to about 1.5:1.0 (capillary whole blood or plasma to venous whole blood or plasma In other aspects, the conversion factor is about 0.8:1.0 (capillary whole blood or plasma to venous whole blood or plasma) to about 1.2:1.0 (capillary whole blood or plasma to venous whole blood or plasma).

Once the conversion factor is selected, the processing further involves normalizing the amount of CK-MB, β-hCG, TSH, homocysteine, and/or free T4 in the capillary whole blood or plasma sample with the amount of the same analyte from venous whole blood or plasma by applying the conversion factor to the amount of analyte (e.g., CK-MB, β-hCG, TSH, homocysteine, and/or free T4) in the sample. For example, the amount of the analyte in the capillary whole blood or plasma sample can be multiplied by the conversion factor to provide the normalized amount of the analyte in the sample.

In some aspects, the processing of the amount of CK-MB, β-hCG, TSH, homocysteine, and/or free T4 (e.g., the result) can be by a processing system which comprises a computer processor and a non-transitory computer memory comprising one or more computer programs, in conjunction with said computer process which are configured to select a conversion factor for comparing the amount of CK-MB, β-hCG, TSH, homocysteine, and/or free T4 in the capillary whole blood or plasma sample with the amount of the same analyte in venous whole blood or plasma and normalize the amount of CK-MB, β-hCG, TSH, homocysteine, and/or free T4 in the sample with the amount of the same analyte from venous whole blood or plasma by applying the selected conversion factor to the amount CK-MB, -hCG, TSH, homocysteine, and/or free T4 in the sample.

This result or processed result can be communicated (e.g., reported) for further analysis, interpretation, processing and/or display. The result can be communicated (e.g., reported) by a computer, in a document and/or spreadsheet, on a mobile device (e.g., a smart phone), on a website, in an e-mail, or any combination thereof. In some aspects, the result can displayed not as a number, but as a visual signal (e.g., a line or bar) that can be read/interpreted by a reader or the naked eye.

In some aspects, the result of CK-MB, β-hCG, TSH, homocysteine, or free T4 or any combination thereof, determined in the sample is communicated by being displayed, such as on an instrument. In further aspects, the result is displayed as indicating that the amount of CK-MB, β-hCG, TSH, homocysteine, or free T4 or any combination thereof, in a subject is elevated, are not elevated, or that the assay(s) for CK-MB, β-hCG, TSH, homocysteine, or free T4 or any combination thereof, should be repeated.

Suitable instruments for use in the methods described herein include a higher throughput assay analyzer (e.g., the ARCHITECT platform marketed by Abbott Laboratories) or a point-of-care device (e.g., i-STAT and i-STAT Alinity devices marketed by Abbott Laboratories) that may contain a user interface that can display the determination.

In some aspects, the result of CK-MB, β-hCG, TSH, homocysteine, or free T4 or any combination thereof, is communicated or capable of being communicated in about 4 minutes to about 40 minutes from the time the sample is collected. In other aspects, the result is communicated or capable of being communicated in about 4 minutes to about 30 minutes from the time the sample is collected. In yet other aspects, the result is communicated or capable of being communicated in about 4 minutes to about 20 minutes from the time the sample is collected. In some aspects, the result is communicated in about 40 minutes or less, about 39 minutes or less, about 38 minutes or less, about 37 minutes or less, about 36 minutes, or less about 35 minutes, less about 34 minutes or less, about 33 minutes or less, about 32 minutes or less, about 31 minutes or less, about 30 minutes or less, about 29 minutes or less, about 28 minutes or less, about 27 minutes or less, about 26 minutes or less, about 25 minutes or less, about 24 minutes or less, about 23 minutes or less, about 22 minutes or less, about 21 minutes or less, about 20 minutes, about 19 minutes, about 18 minutes, about 17 minutes, about 16 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes, or about 4 minutes from the time the sample is collected.

In some aspects in the above method, the time from collection of the sample (from the subject) until the time a result for CK-MB, β-hCG, TSH, homocysteine, free T4, or any combinations thereof is communicated is less than about 30 minutes. In yet other aspects in the above method, the time from collection of the sample until the time a result for CK-MB, β-hCG, TSH, homocysteine, free T4, or any combinations thereof is communicated is less than about 25 minutes. In yet some other aspects in the above method, the time from collection of the sample until the time a result for CK-MB, β-hCG, TSH, homocysteine, free T4, or any combinations thereof is communicated is less than about 20 minutes. n yet still further aspects in the above method, the time from collection of the sample until the time a result for CK-MB, β-hCG, TSH, homocysteine, free T4, or any combinations thereof is communicated is less than about 18 minutes. In some aspects in the above method, the time from collection of the sample until the time a result for CK-MB, β-hCG, TSH, homocysteine, free T4, or any combinations thereof is communicated is less than about 15 minutes. In some aspects in the above method, the time from collection of the sample until the time a result for CK-MB, β-hCG, TSH, homocysteine, free T4, or any combinations thereof is communicated is about 4 to about 20 minutes. In some aspects in the above method, the time from collection of the sample until the time a result for CK-MB, β-hCG, TSH, homocysteine, free T4, or any combinations thereof is communicated is about 15 minutes to about 20 minutes.

In some aspects, the instrument contains software to execute one or more tasks, including the performance of the methods and algorithms described herein. In some aspects, the instrument contains software to automatically determine the next appropriate step in a methods and algorithms as described herein. For example, the instrument may contain software that determines the amount or presence of an analyte of interest. The software may display this determination, such as on a graphical user interface.

In some aspects, the instrument stores software that instructs a processor to execute a given task. In some aspects, the software stores machine readable instructions that instruct a processor to execute a given task. The machine-readable instructions may be one or more executable programs or portion(s) of an executable program for execution by a computer. The programs may be embodied in software stored on a non-transitory computer readable storage medium such as a CD-ROM, a floppy disk, a hard drive, a DVD, a Blu-ray disk, or a memory associated with the processors. Alternatively, the entire programs and/or parts thereof could alternatively be executed by a device other than the processors and/or embodied in firmware or dedicated hardware. Additionally or alternatively, processes may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine-readable instructions may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine-readable instructions may be fragmented and stored on one or more storage devices and/or computing devices (e.g., servers). The machine-readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine-readable instructions may be stored in multiple parts, which are individually compressed, encrypted, and stored on separate computing devices, wherein the parts when decrypted, decompressed, and combined form a set of executable instructions that implement a program such as that described herein.

In another example, the machine-readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine-readable instructions may need to be configured (e.g., settings stored, data input, network addresses recorded, etc.) before the machine-readable instructions and/or the corresponding program(s) can be executed in whole or in part. Thus, the disclosed machine-readable instructions and/or corresponding program(s) are intended to encompass such machine-readable instructions and/or program(s) regardless of the particular format or state of the machine-readable instructions and/or program(s) when stored or otherwise at rest or in transit.

The machine-readable instructions described herein can be represented by any past, present, or future instruction language, scripting language, programming language, etc. For example, the machine-readable instructions may be represented using any of the following languages: C, C++, Java, C#, Perl, Python, JavaScript, HyperText Markup Language (HTML), Structured Query Language (SQL), Swift, etc.

The machine readable instructions may be stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

### 4. Plasma Separation Apparatus

In another embodiment, the present disclosure relates an apparatus that can be used as a plasma separation device. In one aspect, the apparatus comprises a hydrophobic layer comprising at least one microchannel and a top layer that flanks or is positioned above or on top of the hydrophobic layer. The hydrophobic layer can comprise or be constructed from at least one hydrophobic material. The hydrophobic material can be a membrane, film, fabric, fiber, filter, microfilm, screen, mesh, or any combination thereof. In one aspect, the hydrophobic layer is a membrane or film. Hydrophobic membranes or films that can be used are those known in the art. Specifically, membranes or films such as those available from Adhesive Research (Glen Rock, PA), 3M (Minneapolis, Minnesota), and/or Tesa SE (Norderstadt, Germany) can be used.

The hydrophobic layer comprises at least one microchannel having a first and second end which defines a path for capillary fluid flow of a processed blood sample or blood product (e.g., plasma). In some aspects, at least one microchannel extends longitudinally along a portion of the hydrophobic layer from the first end to an opening at the second (e.g., opposite) end of the microchannel. In other aspects, the at least one microchannel extends the width of a portion of the hydrophobic layer from the first end to an opening at the second (e.g., opposite) end of the microchannel. In some aspects, the microchannel contains a first opening connected to the first end of the microchannel. In these aspects, the processed blood or blood product (e.g., plasma) can flow from the first opening into the first end of the microchannel to the second opening at the second end of the microchannel.

The opening at the second end of the microchannel allows the processed blood or blood product (e.g., plasma) to flow out of the apparatus. For example, if the fluid being processed is blood, plasma can flow from the first end towards the second opening at the second (e.g., opposite) end by capillary fluid flow. The plasma can be collected at the second (e.g., opposite) end using a collection or other device or, if the apparatus is operably linked, removably coupled, or in fluid communication with another device (such as a sample analysis cartridge (such as a microfluidic cartridge)), allowed to continue to flow directly on or into the device for further processing and/or analysis.

The microchannel can be of any length. In some aspects, the microchannel is less than about 80 mm in length. In yet other aspects, the at least one microchannel is about 70 mm in length, the at least one microchannel is about 60 mm in length, about 55 mm in length, about 50 mm in length, about 45 mm in length, about 40 mm in length, about 35 mm in length, about 30 mm in length, about 25 mm in length, about 20 mm in length or about 15 mm in length. In yet other aspects, the at least one microchannel is less than about 5 mm wide, less than about 4.5 mm wide, less than about 4 mm wide, about 3 mm wide, less than about 2.5 mm wide, or less than about 2.0 mm wide.

Additionally, the at least one microchannel can be positioned at any location on the hydrophobic layer. For example, the at least one microchannel can be centered on the hydrophobic layer, it can be slightly off center on the hydrophobic layer, or it can be at or close to a side or edge of the hydrophobic layer. In yet other aspects, the microchannel does not contain a filter or any type of filtering device (e.g., the microchannel does not contain a filter or filtering device at any point between its first and second end which defines the path for capillary fluid flow of the processed blood sample or blood product). Yet alternatively, in some aspects, the microchannel contains one or more filters or filtering type devices.

In yet additional aspects, the hydrophobic layer can have a thickness of about 50 to about 200 microns. In yet other aspects, the hydrophobic layer can have a thickness of about 100 to about 200 microns. In some aspects, the hydrophobic layer can have a thickness of about 100 to about 150 microns.

The apparatus also comprises a top layer that flanks or is positioned above or on top of the hydrophobic layer. In some aspects, the top layer is adherent to the hydrophobic layer. In yet further aspects, the surface of the top layer that faces the hydrophobic layer comprises a material or is coated with a hydrophilic material. The hydrophilic material can be an adhesive, membrane, film, fabric, fiber, filter, microfilm, screen, mesh, or any combination thereof. In other aspects, the entire top layer is made or constructed of a hydrophilic material such as a membrane, film, fabric, fiber, filter, microfilm, screen, mesh, or any combination thereof. For example, in one aspect, the top layer is a membrane or film. In some aspects, the membrane or film is not constructed from a hydrophilic material but is coated with a hydrophilic material. The portion of the top layer coated with the hydrophilic material faces the hydrophobic layer. In another aspect, the entire membrane or film is made or constructed from a hydrophilic material. Hydrophilic membranes or films that can be used are those known in the art. An example of hydrophilic film that can be used is 9984 Diagnostic Microfluidic Surfactant Free Hydrophilic Film available from 3M (Minneapolis, MN), Kemafoil H, Hydrophilic coated polyester film from Coveme (S. Lazzaro diSavena, Italy), Tesa 62580, Hydrophilic coated polyester film from Tesa SE (Norderstadt, Germany). In yet additional aspects, the top layer can have a thickness of about 50 to about 200 microns. In yet other aspects, the top layer can have a thickness of about 100 to about 200 microns. In some aspects, the top layer can have a thickness of about 100 to about 150 microns.

When a blood sample or blood product is placed on the top layer of the apparatus, the blood sample or product flows through the top layer. As it does so, cellular components of the blood (e.g., red blood cells, white blood cells, platelets, and combinations thereof) are captured in the pores and/or fibers of the hydrophilic material thereby allowing the plasma to continue to flow through the hydrophilic material to the hydrophobic layer and into the microchannel. Once in the microchannel, the plasma flows by capillary fluid flow from the first end to the opening at the second (e.g., opposite) end of the channel. The plasma can be collected using a collection or other device, or, if the apparatus is operably linked, removably coupled, or in fluid communication with another device (such as a sample analysis cartridge (e.g., a microfluidic cartridge), allowed to continue to flow directly on or into the device for further processing and analysis.

In another aspect, the apparatus optionally comprises a bottom layer. The bottom layer is flanked or is positioned below or beneath the hydrophobic layer. In these aspects, the apparatus comprises at least three layers-a top layer, a hydrophobic layer, and a bottom layer. In some aspects, the bottom layer is adherent to the hydrophobic layer. In additional aspects, the top layer and bottom layer are each adherent to the hydrophobic layer.

In yet further aspects, the surface of the bottom layer that faces the hydrophobic layer comprises a material or is coated with a hydrophilic material. The hydrophilic material can be an adhesive, membrane, film, fabric, fiber, filter, microfilm, screen, mesh, or any combination thereof. In other aspects, the entire bottom layer is made or constructed from a hydrophilic material such as a membrane, film, fabric, fiber, filter, microfilm, screen, mesh, or any combination thereof. For example, in one aspect, the bottom layer is a membrane or film. In some aspects, the membrane or film is not constructed from a hydrophilic material but is coated with a hydrophilic material. The portion of the bottom layer coated with the hydrophilic material faces the hydrophobic layer. In another aspect, the entire membrane or film is made or constructed from a hydrophilic material. Hydrophilic membranes or films that can be used are those known in the art. An example of hydrophilic film that can be used is 9984 Diagnostic Microfluidic Surfactant Free Hydrophilic Film available from 3M (Minneapolis, MN), Kemafoil H, Hydrophilic coated polyester film from Coveme (S. Lazzaro diSavena, Italy), Tesa 62580, Hydrophilic coated polyester film from Tesa SE (Norderstadt, Germany).

In yet additional aspects, the bottom layer can have a thickness of about 50 to about 200 microns. In yet other aspects, the bottom layer can have a thickness of about 100 to about 200 microns. In some aspects, the bottom layer can have a thickness of about 100 to about 150 microns.

In still further aspects, the apparatus can contain a protective film which flanks or is positioned below or beneath the bottom layer. In some aspects, the protective film is adherent to the bottom layer. The protective film protects the apparatus from moisture and/or other contamination. The protective film can be a plastic film, such as a self-adhesive plastic film, cardboard with adhesive, a plastic sheet with adhesive, or combinations thereof.

In yet other aspects, when the apparatus contains a top layer, a hydrophobic layer and a bottom layer, the combined thickness of the three layers is between about 100 to about 600 microns. In other aspects, the combined thickness of the three layers is between about 150 to about 600 microns. In other aspects, the combined thickness of the three layers is between about 200 to about 600 microns. In yet other aspects, the combined thickness of the three layers is between about 150 to about 500 microns. Still yet further aspects, the combined thickness of the three layers is between about 200 to about 500 microns.

Additionally, in some aspects, when the apparatus contains a top layer, a hydrophobic layer and a bottom layer, the top layer and the bottom layer can be made from the same material. Alternatively, the top layer and the bottom layer can be made from different materials. For example, the top layer and bottom layer can be made or constructed from the same hydrophilic material or different hydrophilic materials. Alternatively, the top and bottom layers may be made or constructed from a material that is not hydrophilic but are coated with a hydrophilic material on the surface of the layer that faces the hydrophobic layer. Still further, either one of the top or bottom layers may be made or constructed from a material that is not hydrophilic and coated with a hydrophilic material on the surface facing the hydrophobic layer and the other layer made entirely from a hydrophilic material.

In some aspects, the top layer comprises a sample inlet where the blood sample or blood product is placed to begin the sample processing through the apparatus. The sample inlet can have any shape. For example, the sample inlet can be round, oval, rectangular, square, triangular, or any combination thereof. In some aspects, a hydrophobic transfer material (e.g., such as a transfer tape) can surround the sample inlet. The hydrophobic transfer material helps prevent the blood sample or blood product from wicking or moving away from the sample inlet area.

When the top layer comprises a sample inlet, the hydrophobic layer and optionally, the bottom layer, comprise one or more openings. The openings can have any shape. For example, the opening can be round, oval, rectangular, square, triangular, or any combination thereof. The opening can have the same shape as the sample inlet or it can have a different shape. In some aspects, when the apparatus contains two layers (e.g., a top layer having a sample inlet and a hydrophobic layer), one or more openings can be made in the hydrophobic layer. In yet other aspects, when the apparatus contains three layers, one or more openings can be made in the hydrophobic layer but not in the bottom layer. In other aspects, when the apparatus contains three layers, one or more openings can be made in each of the hydrophobic layer and bottom layer. Each opening in the one or more layers can be directly below the sample inlet.

In some aspects, the apparatus described herein is used to separate plasma from whole blood in a horizontal orientation. In other aspects, the apparatus is used to separate plasma from whole blood in vertical orientation. When the apparatus is used vertically, when loading, the microchannel can be bent 90 degrees (e.g., keeping the membrane horizontal and channel vertical), or alternatively, a closed inlet with a funnel like structure can be used.

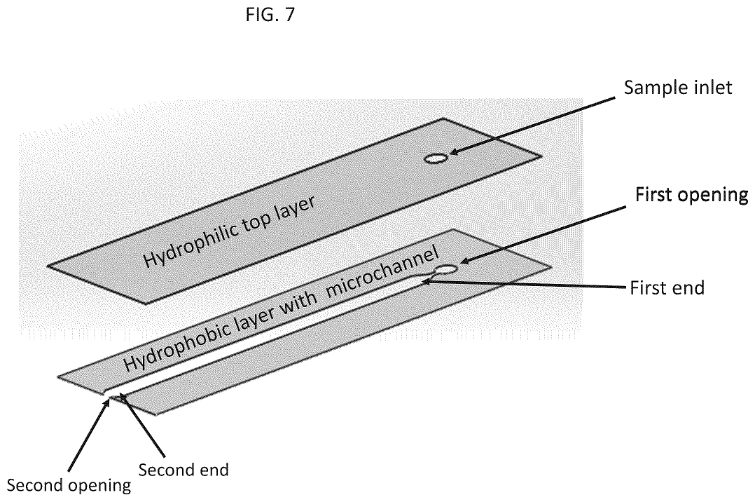

For example, FIG. 7 illustrates one embodiment of the apparatus of the present disclosure, which is being used in a horizontal orientation, which contains a hydrophilic top layer and a hydrophobic layer containing a microchannel having a first and second end. The top layer is adherent to the hydrophobic layer. The hydrophilic top layer comprises a sample inlet and the hydrophobic layer contains a first opening directly below the sample inlet. The first opening in the hydrophobic layer directly below the sample inlet is connected to the first end of the microchannel. When the plasma reaches the first opening on the hydrophobic layer, it flows from the opening into the first end of the microchannel and continues to flow to the second opening at the second end (e.g., opposite end) where it can be collected or allowed to flow directly on or into a sample analysis cartridge.

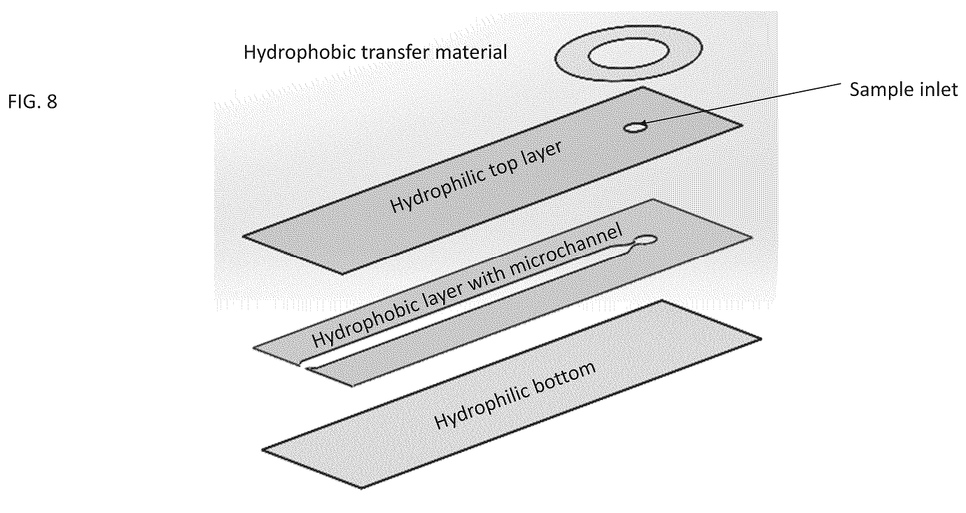

FIG. 8 illustrates another embodiment of the apparatus of the present disclosure, which is being used in a horizontal orientation. In this embodiment, the apparatus comprises a hydrophilic top layer, a hydrophobic layer and a hydrophilic bottom layer. The hydrophilic top layer is adherent to the hydrophobic layer and the bottom layer is adherent to the hydrophobic layer. The hydrophilic top layer comprises a hydrophobic transfer tape and a sample inlet. The hydrophobic layer contains a first opening directly below the sample inlet. The first opening in the hydrophobic layer directly below the sample inlet is connected to the first end of the microchannel. When the plasma reaches the first opening on the hydrophobic layer, it flows into the first end of the microchannel and continues to flow to the second opening at the second (e.g., opposite) end where it can be collected or allowed to flow directly on or into a sample analysis cartridge.

In still further aspects, the sample inlet can further comprise a separation membrane (such as a plasma separation membrane). In some aspects, the separation membrane is a glass fiber material, such as a membrane, film, fabric, fiber, filter, microfilm, screen, mesh, or any combination thereof. Additionally, the separation membrane can be in any shape. For example, the sample inlet can be round, oval, rectangular, square, triangular, or any combination thereof. In some aspects, the separation membrane can have the same shape as the sample inlet. In other aspects, the separation membrane can have a different shape than the sample inlet.

The separation membrane can be made from any material known in the art to be useful for separating blood samples or blood products into its components, such as plasma. When a blood sample or blood product is placed on a separation membrane, the blood sample or product flows through the membrane or material. As it flows through the membrane or material, cellular components of the blood (e.g., red blood cells, white blood cells, platelets, and combinations thereof) are captured in the pores and/or fibers of the membrane or material thereby allowing the plasma to continue to flow through the sample inlet and to the first opening on the hydrophobic layer. The first opening on the hydrophobic layer is connected to the microchannel such that when the plasma reaches the first opening on the hydrophobic layer, it flows into the first end of the microchannel and continues to flow to the second opening at the second (e.g., opposite end) of the microchannel. Once the plasma reaches the second opening at the second end of the microchannel, it can be collected using a collection or other device, or, if the apparatus is operably linked, removably coupled, or in fluid communication with another device (such as a sample analysis cartridge (e.g., a microfluidic cartridge), allowed to continue to flow directly on or into the device for further processing and analysis.

In some aspects, once the plasma reaches the first opening, second opening, or both the first and second opening of the microchannel, a pump, such as an air pump, foam ring pump, foil blister pump, film blister pump, or any combination thereof can be used to direct and/or disperse the plasma in the microchannel. In some aspects, the pump can be used to direct and/or disperse the plasma from the first end of the microchannel towards the second end of the microchannel. In yet other aspects, the pump can be used to direct and/or disperse the plasma from the second end of the microchannel towards and/or into another device (such as a sample analysis cartridge (e.g., a microfluidic cartridge). The pump can be connected at any point along the microfluidic channel, such as at the first opening, along the side, near the second end, or any combination thereof.

In some aspects, the separation membrane can be positioned or placed above, below or within the sample inlet. For example, in some aspects, the separation membrane can be cut to be larger than the size of the sample inlet and simply lay on top of the sample inlet. Alternatively, the separation membrane can be cut to be the same size or slightly smaller than the size of the sample inlet and be placed in the inlet. Still further, the separation membrane can be placed underneath the sample inlet and attached or adhered by any means known in the art, such as by an adhesive, glue, etc.

When the hydrophobic layer and/or bottom layer contain one or more openings, the hydrophobic layer and/or bottom layer can further contain one or more separation membranes positioned or placed above, below or within the opening in the layer. The separation membrane can be constructed from the same materials as used for the sample inlet or can be constructed from different materials. In still further aspects, the separation membrane can be positioned or placed above, below or within one or more openings. For example, in some aspects, the separation can be cut to be larger than the size of the opening and simply lay on top of the opening. Alternatively, the separation membrane can be cut to be the same size or slightly smaller than the size of the opening and be placed in the inlet. Still further, the separation membrane can be placed underneath the opening and attached or adhered by any means known in the art, such as by an adhesive, glue, etc.

In yet further aspects, one or more hydrophilic meshes or hydrophilic films can flank the separation membrane at the sample inlet and/or one or more openings. For example, a hydrophilic mesh or hydrophilic film can be positioned above or on top of the separation membrane to facilitate the spread of the blood sample or blood product into the apparatus. Alternatively, a hydrophilic mesh or hydrophilic film can be positioned below or beneath the separation membrane to help facilitate the continued movement of the blood sample or blood product as it is processed through to the hydrophobic layer and the first opening that is connected to the first end of the microchannel.

In some aspects, the apparatus further comprises an upper substrate material that comprises a sample inlet. The upper substrate material flanks or is positioned above or on top of the top layer. In some aspects, the top layer is adherent to the upper substrate. The upper substrate material can be a membrane, film, fabric, fiber, filter, microfilm, screen, mesh, or any combination thereof. In some aspects, the upper substrate material is made of a hydrophilic material. In other aspects, the upper substrate material is made from a hydrophobic material.

The upper substrate material comprises a sample inlet where the blood sample or blood product is placed to begin the sample processing. The sample inlet can have any shape. For example, the sample inlet can be round, oval, rectangular, square, triangular, or any combination thereof. In some aspects, a hydrophobic transfer material (e.g., such as a transfer tape) can surround the sample inlet. The hydrophobic transfer material helps prevent the blood sample or blood product from wicking or moving away from the sample inlet area.

In still further aspects, the sample inlet can comprise a separation. In some aspects, the separation membrane comprises a glass fiber material such as a membrane, film, fabric, fiber, filter, microfilm, screen, mesh, or any combination thereof. The separation membrane can be made from any material known in the art to be useful for separating blood samples or blood products into its components, such as plasma. The separation membrane in the upper substrate functions in the same way as the separation membrane used in the sample inlet of the top layer.

In some aspects, the separation membrane can be positioned or placed above, below or within the sample inlet. For example, in some aspects, the separation membrane can be cut to be larger than the size of the sample inlet and simply lay on top of the sample inlet. Alternatively, the separation membrane can be cut to be the same size or slightly smaller than the size of the sample inlet and be placed in the inlet. Still further, the separation membrane can be placed underneath the sample inlet and attached or adhered by any means known in the art, such as by an adhesive, glue, etc.

When an upper substrate and sample inlet are present, one or more openings are made in each of the top layer and hydrophobic layer or each of the top layer, hydrophobic layer and optionally, the bottom layer. The opening can have any shape. For example, the opening can be round, oval, rectangular, square, triangular, or any combination thereof. In some aspects, the opening is the same shape as the sample inlet. In other aspects, the opening is a different shape as the sample inlet. In some aspects, when the apparatus contains an upper substrate, one or more openings can be made in the top layer and hydrophobic layer but not in the bottom layer. In other aspects, one or more openings can be made in each of the top layer, hydrophobic layer and bottom layer. Each opening in the one or more layers can be directly below the sample inlet. One or more openings in each of the top layer, hydrophobic layer and/or bottom layer can also contain one or more separation membranes positioned or placed above, below or within the opening in the layer. The separation membrane can be constructed from the same materials as used for the sample inlet or can be constructed from different materials. Additionally, one or more hydrophilic meshes or films can flank the separation membrane at the sample inlet and/or one or more openings. For example, a hydrophilic mesh or hydrophilic film can be positioned above or below the separation membrane as described previously herein.

In still another aspect, the top layer, hydrophobic layer, bottom layer, hydrophilic mesh or hydrophilic film, separation membrane, or any combination thereof is either ubiquitous for any analyte or specific for an analyte or class of analytes.

It was found that the volume of plasma that is generated by the apparatus depends on the hematocrit (% PCV) of the whole blood sample). Specifically, due to the short vertical separation distance in the apparatus, at higher hematocrits, the volume of the generated plasma can be lower than the volume of the microchannel. Plasma flows into the microchannel by capillary action but if the volume of plasma is not enough to fill the entire channel, then red blood cells held back by the separation membrane fill the remainder. Thus, as a result, in some aspects, the whole blood sample used with the apparatus has a hematocrit of less than about 40, less than about 39, less than about 38, less than about 37, less than about 36, less than about 35, less than about 34, less than about 33, less than about 32, less than about 31, less than about 30, less than about 29, less than about 28, less than about 27, less than about 26, less than about 25, less than about 24, less than about 23, less than about 22, less than about 21, less than about 20, less than about 19, less than about 18, less than about 17, less than about 16, or less than about 15. In some aspects, the whole blood sample used with the apparatus has a hematocrit of between about 15 to about 40. In other aspects, the whole blood sample used with apparatus has a hematocrit of between about 20 to about 35. In still other aspects, the whole blood sample used with the apparatus has a hematocrit of between about 25 to about 35.

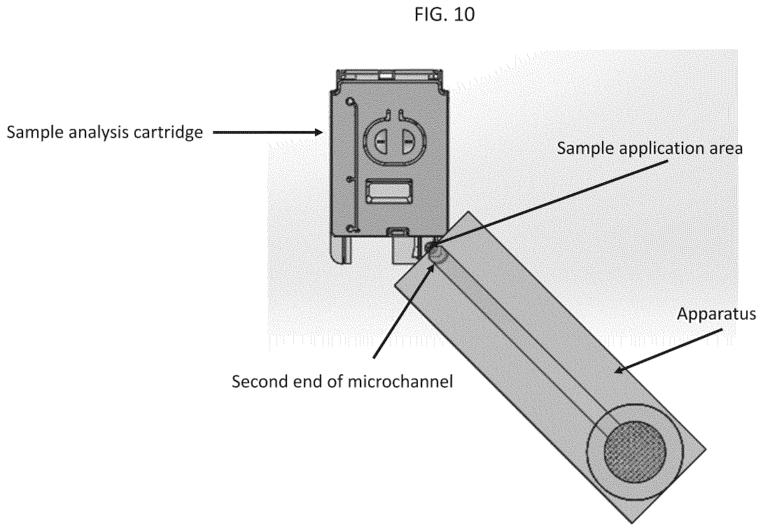

In still another aspect, the apparatus described herein, such as, for example, the apparatus shown in FIGS. 7-9, can be used in connection with a point-of-care device, such as shown, for example, in FIG. 10. The apparatus can also be optimized for use with a non-point-of-care device, such as a high throughput analyzer (e.g., a non-point-of-care device), using routine techniques known in the art. Such optimization could include increasing one or more of the length, width and/or thickness of the apparatus. For example, in some aspects, the apparatus can have a thickness of about 600 microns to about 10,000 microns. Additionally, in other aspects, the length and/or width of the microchannel can be increased. For example, the length of the microchannel can be from about 80 mm to about 200 mm in length and/or the width of the microchannel can be from about 5 mm to about 15 mm. In some aspects, the apparatus comprises one (e.g., a single) microchannel. In other aspects, the apparatus can contain more than one microchannel. For example, the apparatus can contain at least 2 microchannels, at least 3 microchannels, at least 4 microchannels, or at least 5 microchannels.

The apparatus described herein, such as, for example, the apparatus shown in FIGS. 7-9, is a passive membrane-based separator. As described herein, whole blood can be applied on top of the membrane. A hydrophilic fabric enables a more even wetting on the top of the membrane and directs the blood to travel through the separation media vertically. Cell components are retained in the membrane matrix which generates an advancing plasma front. An uniform, almost column like travel of blood through the separation media maximizes the volume of plasma generated at the bottom of the membrane. Once the separation membrane is saturated, the plasma from the bottom of the membrane is directed into a microchannel by capillary action.

In still another aspect, the apparatus further comprises at least one agglutinating agent to agglutinate the red blood cells to form red blood cell aggregates to improve separation and produce a cleaner plasma. In some aspects, the agglutinating agent is coated or incorporated into or on one or more of the top layer, hydrophobic layer, bottom layer, upper substrate, separation membrane, hydrophilic mesh or hydrophilic film, or any combination thereof. Examples of an agglutinating agent that can be used includes, lectin (e.g., soybean lectin), Merquat-100, Concanavalin A, DEAE-Dextran, poly-L-lysine, polyvinylpyrrolidone, poly(2-(dimethylamino)ethylmethacrylate), or any combinations thereof.

In addition, the apparatus can further comprise one or more salts which can be used to achieve least a partial crenation of the red blood cells in the whole blood sample. Salts are ionic compounds that dissociate in water. Salts that can be used in the apparatus include both organic and inorganic salts. Examples of salts that can be used include, for example, calcium chloride, potassium chloride, sodium chloride, manganese chloride, magnesium chloride, potassium sulfate, guanidine hydrochloride and combinations thereof. In still yet another aspect, the top layer, hydrophobic layer, bottom layer, upper substrate, separation membrane, hydrophilic mesh or hydrophilic film, or any combination thereof, is coated with a coating, such as with a surfactant, hydrophilic coating or any combination thereof, to improve or increase the speed or rate of separation of the plasma and/or serum from whole blood as it passes through any of the layers, substrates, membranes, meshes, files, or any combination thereof.

The plasma produced using the apparatus disclosed herein may not be pure plasma but rather plasma depleted of one or more components of blood (e.g., red blood cells, white blood cells, platelets, and combinations thereof). In some aspects, the plasma contains about 5% by volume or less of red blood cells, white blood cells and/or platelets. In other aspects, the plasma contains about 5% or less by volume of red blood cells. In other aspects, the plasma contains about 4% or less by volume of red blood cells. In yet other aspects, the plasma contains about 3% or less by volume of red blood cells. In yet other aspects, the plasma contains about 2% or less by volume of red blood cells. In still yet further aspects, the plasma contains about 1% or less by volume of red blood cells. In other words, in some aspects, the product produced by the apparatus described herein is a product that is reduced in the number of blood cells (e.g., the product is red cell reduced) when compared to a whole blood sample or blood product that has not been contacted with the apparatus described herein.

In another embodiment, the present disclosure relates to a device. In one aspect the device comprises the apparatus described previously herein and at least one sample analysis cartridge (e.g., a microfluidic cartridge) having a sample application area where a test sample is applied. In one aspect, the apparatus is configured with the sample analysis cartridge such that the microchannel is operably linked, removably coupled, or in fluid communication with the sample application area. Specifically, as shown in FIG. 10, the second end of the microchannel can be in operably linked, removably coupled, or in fluid communication with a sample analysis cartridge at a sample application area. In another aspect, the apparatus is included or incorporated as part of a clip, such as a moby clip, extended moby clip, etc., and configured, coupled, or integrated with the sample analysis cartridge such that the microchannel is operably linked, removably coupled, or in fluid communication with the sample application area.

In another aspect, the plasma separation apparatus comprises a pre-evacuated container or tube, having an inlet and outlet end, each of which are closed. A pressure differential exists between the inlet and outlet ends of the container or tube. The inlet end of the container or tube comprises a cap or septum which is capable of being penetrated by a needle or blood-collection needle assembly used to obtain a whole blood sample from a subject. The outlet end comprises a serum holding chamber (e.g., a filtrate container) which receives the serum and/or plasma (e.g., filtrate) produced by the apparatus.

The inlet end of the container or tube containing the cap or septum that is adapted for being pierced by a needle or standard blood-collecting needle assembly defines a first end of a blood holding chamber that is free to accept the whole blood sample from the needle or blood-collection needle assembly for filtration.

In some aspects, a filter assembly is adjacent to the second end of blood holding chamber. The filter assembly captures the cellular components of the blood (e.g., red blood cells) and allows the passage of the serum and/or plasma components through the assembly to a serum holding chamber at the outlet end. In some aspects, the filter assembly covers the entire cross-section area of the container or tube. In some aspects, the filter assembly permits the passage of particles or molecules smaller than about 0.7 microns, about 0.6 microns, about 0.5 microns, about 0.4 microns, or about 0.3 microns, and functions similar to size-exclusion chromatography (SEC, also known as gel filtration) where smaller sized particles and molecules (e.g., serum and/or plasma) pass through the filter assembly faster than larger sized molecules (e.g., red blood cells). The filter assembly can be made of any material or combination of materials that can be used to separate the components of blood based on size and allow the passage of serum and/or plasma components through the assembly and towards the serum holding chamber. For example, in some aspects, the filter assembly comprises one or more microfiber membranes and/or glass fiber filter materials (e.g., such as low-density fiber filter material). For example, Micro-Strand Glass Microfibers from Johns Manville (Fruita, CO) can be used. In some aspects, the highly hydrophilic, highly porous material such as those available from Porex Filtration Group (South Chesterfield, VA) such as, POR 410 or POR4711, can be used in the filter assembly. In yet further aspects, the filter assembly terminates and is retained and supported near the middle of the container or tube with a screen member or other perforated material.

Optionally, in some aspects, a flow regulator may be placed adjacent to the cap or septum and can be used to regulate the rate of flow of the blood to the filter assembly. In other aspects, the flow regulator can be included as part of blood-collection needle assembly.

Because the container or tube is pre-evacuated, a pressure differential exists at the area around the filter assembly. The pressure at the top of the container or tube in the blood holding chamber is higher than the pressure at the bottom of the container at the serum holding chamber. Due to this pressure differential, the whole blood sample in the blood holding chamber moves towards and through the filter assembly where the larger red blood cells are captured and entangled in the filter assembly and the serum and/or plasma (e.g., filtrate) move through the filter assembly at a much faster rate to the serum holding chamber where it is collected.

The serum holding chamber has a hollow space sized to hold the serum and/or plasma resulting from the filter assembly. The serum holding chamber is removably attached (e.g., detachable) to the container or tube and is capable of being detached from the container or tube to allow further processing and/or analysis of the serum or plasma. For example, the serum holding chamber can be detached from the container or tube by twisting or sliding the serum holding chamber in a clockwise or counter-clockwise motion to break the vacuum. Once the serum holding chamber is detached from the container or tube, the serum or plasma sample can be used for further processing and/or analysis on another device and/or apparatus. For example, the serum or plasma sample in the serum holding chamber can be pipetted into another container or device (e.g., a cartridge) for further processing and/or analysis.

In other aspects, the plasma separation can occur in a container or tube that is not pre-evacuated. In such aspects, plasma separation can be performed using any appropriate means, e.g., any means including employing size exclusion-type chromatography to separate molecules or the components in blood by differences in size as they pass through a material (e.g., filter assembly) contained within the container or tube. For instance, any material that would help preferentially slow down the movement of the red blood cells as compared to the plasma such that plasma can be retrieved could be employed (e.g., glass or porous beads, membranes, one or more filter, glass or other fiber materials, or any combination thereof.). Such methods would include using other than a pressure differential, for instance, gravity-fed separation could be employed without the use of any pressure differential.

An example of pre-evacuated container or tube having the above-described components which can be used as a plasma separation apparatus in the methods described in the present disclosure include those described in U.S. Pat. No. 9,427,707, the contents of which are herein incorporated by reference.

In yet other aspects, the above described plasma separation devices can be used in connection with a capillary blood sample which is collected (1) in a decentralized setting; (2) without the use of a syringe, standard needle, or combination thereof; (3) by a user not trained in collecting blood samples from a subject; (4) by a robot; (5) by a self-or other-administered blood collection device; or (6) any combination thereof.

In still further aspects, the above-described plasma separation devices can be used in connection with a blood sample obtained from a subject that is collected with the use of a syringe, standard needle, or combination thereof. Such a sample may be collected in a decentralized or centralized setting (e.g., in a traditional medical setting (such as a hospital, physician office, stand alone lab site, etc.), by a user trained in collecting blood or a combination thereof. In some aspects, the blood sample obtained from a subject that is collected with the use of a syringe, standard needle, or combination thereof is a venous blood sample. In other aspects, the blood sample obtained from a subject that is collected with the use of a syringe, standard needle, or combination thereof is a capillary blood sample. For example, in some aspects, a blood sample (e.g., a venous blood sample and/or a capillary blood sample) can be obtained from a subject using a syringe, standard needle, or combination thereof (and optionally, in a centralized setting and/or by a user trained in collecting blood samples from a subject) and -processed prior to performing an assay for UCH-L1, GFAP, CK-MB, β-hCG, TSH, homocysteine, free T4 and/or any combinations thereof, using the above described plasma separation devices.

5. Systems for Determining the Amount of UCH-L1, GFAP, CK-MB, β-hCG, TSH, Homocysteine, Free T4 or any Combinations Thereof using a Capillary Blood Sample

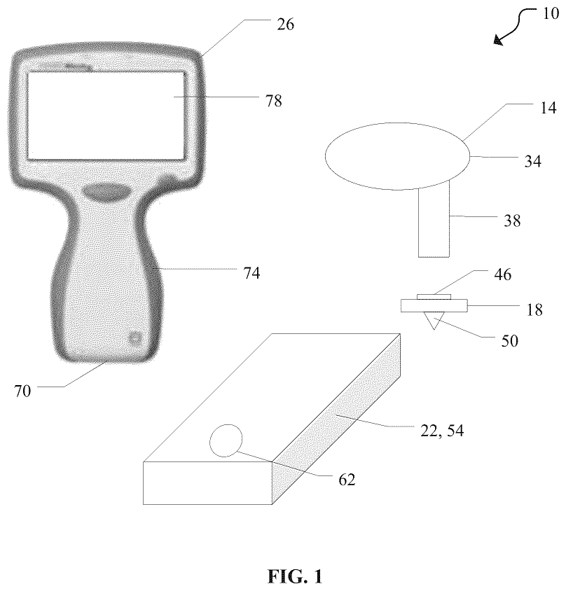

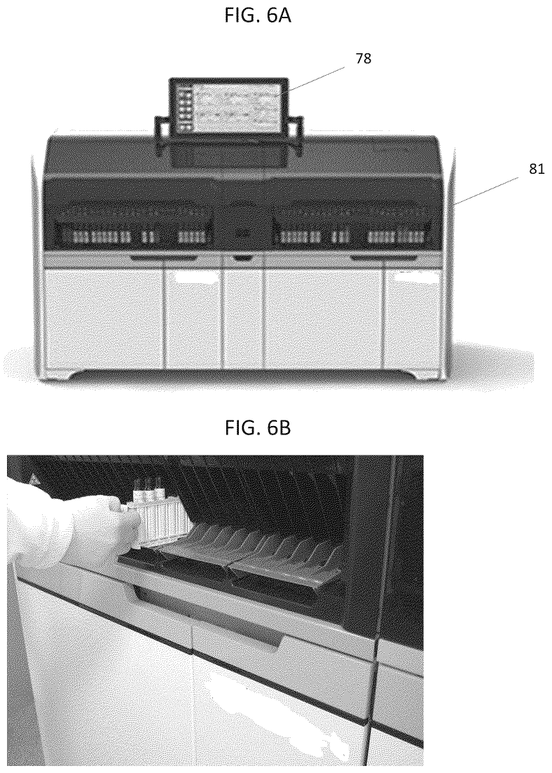

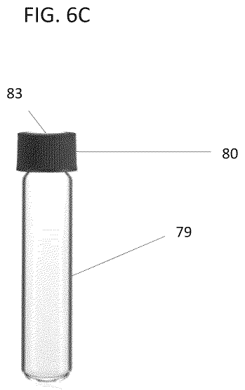

With reference to FIG. 1, a system 10 for determining the amount of UCH-L1, GFAP, CK-MB, β-hCG, TSH, homocysteine, free T4 or a any combinations thereof is provided. The amount of UCH-L1, GFAP, CK-MB, β-hCG, TSH, homocysteine, free T4 or any combinations thereof can be used to aid in the diagnosis and/or evaluation of a subject that has sustained or may have sustained an injury to the head. In one aspect, the system 10 includes a microsampling device 14, a reaction vessel 22, and an instrument, such as instrument 26 (e.g, a point-of-care device). In other aspects, the system may use instrument 81 (e.g., a higher throughput analyzer as shown in FIG. 6A), instead of instrument 26. It should be noted that although the shape of the reaction vessel 22 shown in FIG. 1 is rectangular, the shape is not critical. For example, in some aspects, reaction vessel 22 may be in the shape of a tube. In further aspects, the reaction vessel 22 can be a microfluidic cartridge.

In some additional aspects, the system can further include a plasma separation device, 18 (FIG. 1). When the plasma separation device 18 is present, the microsampling device 14 collects a capillary blood sample from a subject, and the plasma separation device 18 creates a processed capillary blood sample (e.g., serum or plasma) from the capillary blood sample.

In yet further additional aspects, the system can further include a transfer tube, 79 (FIG. 6C). The transfer tube 79 can include a cap or stopper 80. The transfer tube 79 also has an aperture 83. The aperture 83 allows the reaction tube to receive a capillary blood sample or processed capillary blood sample.

In some aspects, the reaction vessel 22 or transfer tube 79 receives the processed capillary blood sample from the plasma separation device 18.

The instrument 26 or 81 analyzes the reaction vessel 22 to provide a determination of the amount of a subject's UCH-L1, GFAP, CK-MB, β-hCG, TSH, homocysteine, free T4 or any combinations thereof. In some aspects, the determination of the amount is communicated as a result. This result can be communicated for further analysis, interpretation, processing and/or displaying. The amount can be communicated by a computer, in a document and/or spreadsheet, on a mobile device (e.g., a smart phone), on a website, in an e-mail, or any combination thereof.

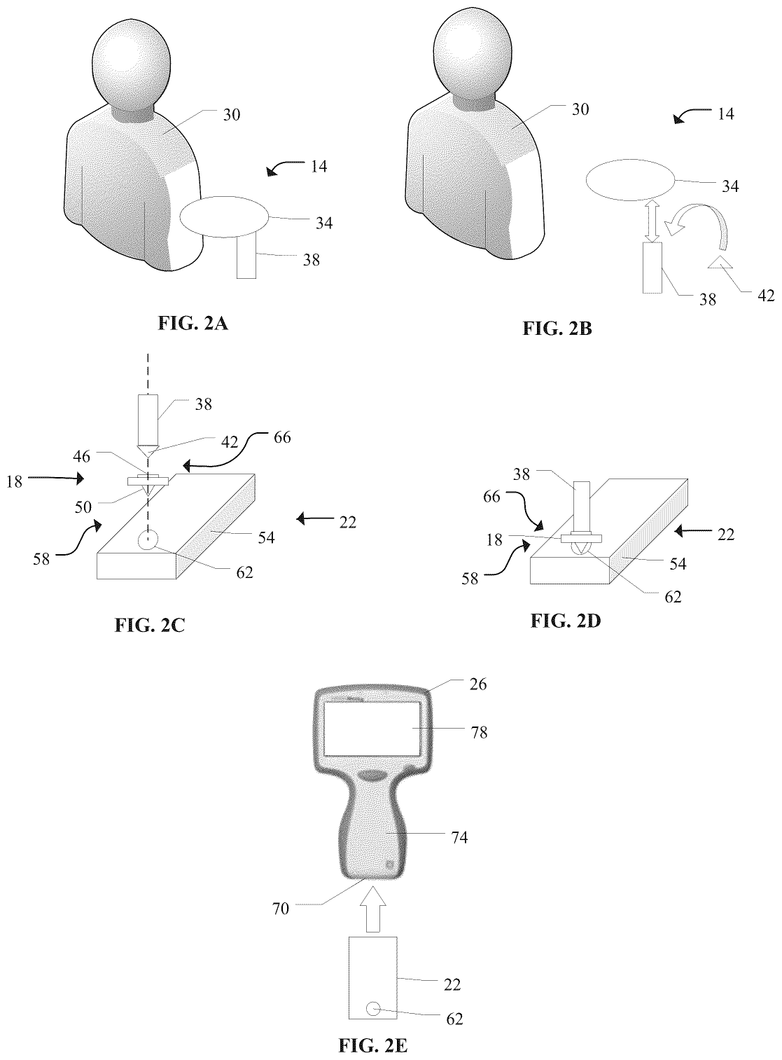

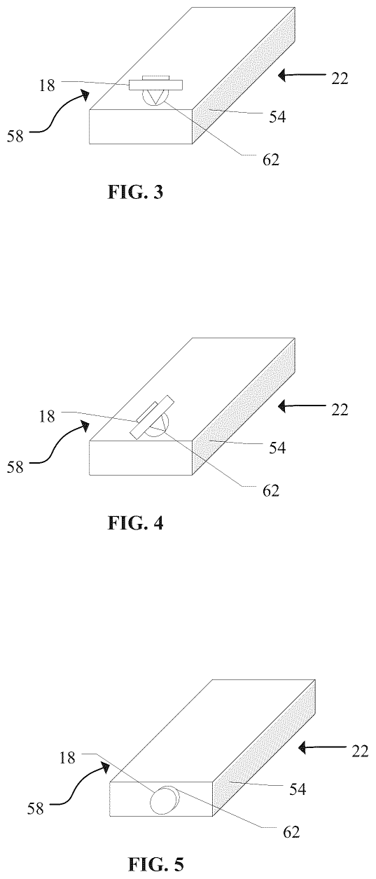

In some aspects, the communicated amount of UCH-L1, GFAP, CK-MB, β-hCG, TSH, homocysteine, free T4 or any combinations thereof can be displayed, such as on an instrument, such as instrument 26 or 81. For example, the amount of UCH-L1, GFAP, CK-MB, β-hCG, TSH, homocysteine, free T4 or any combinations thereof can be displayed as being elevated, not elevated, or that the test (e.g., assay) should be repeated). Various operating steps with the system 10 are illustrated with respect to FIGS. 2A-2E and FIG. 3A-3C are described in further detail herein.

With reference to FIG. 2A, the microsampling device 14 is coupled to a subject 30 and a capillary blood sample is drawn from the subject 30. In the illustrated embodiment, the microsampling device 14 includes a housing 34 and a receptacle 38 coupled to the housing 34. In the illustrated embodiment, the capillary blood sample is collected in the receptacle 38. In other aspects, the receptacle 38 is removably coupled to the housing 34. For example, the receptacle 38 may be separated from the housing 34 once the capillary blood sample is collected. In still yet other aspects, the receptacle 38 is the reaction vessel 22. In these aspects, when the receptacle is the reaction vessel, the receptacle can be removed from the microsampling device 14 and inserted directly into instrument 26 or 81.

The microsampling device 14 further includes a microneedle, a lancet, a microlancet, a blade, a microblade, a microscrew, or any combination thereof coupled to the housing. In some aspects, the microsampling device 14 includes a plurality of microneedles. In some aspects, the microsampling device 14 further includes an actuator movable relative to the housing 34. The actuator may actuate the microneedles or similar components into a subject's skin to begin drawing a capillary blood sample from the subject 30.

With reference to FIG. 2B, the system 10 may further include a cap 42 coupled to the receptacle 38. In some embodiments, the cap 42 is attached to the receptacle 38 after the receptacle is removed from the housing 34. In aspects, the cap 42 seals the capillary blood sample within the container 38. For example, a threaded configuration may couple the cap 42 with the receptacle 38. In some embodiments, the cap 42 is part of an interface between the receptacle 38 and the plasma separation device 18.

With reference to FIG. 2C, the plasma separation device 18 includes an inlet 46 to receive the capillary blood sample from the microsampling device 14 and an outlet 50 through which the processed capillary blood sample leaves the plasma separation device 18. In the illustrated embodiment, the inlet 46 receives the capillary blood sample from the receptacle 38. In some aspects, the plasma separation device 18 is integrated within the receptacle 38 (e.g., the plasma separation device 18 is not intended to be removed from the receptacle 38 by the user).

In some other aspects, the plasma separation device 18 is separately formed from the reaction vessel 22 or transfer tube 79 (i.e., the plasma separation device 18 is movable and can be separated from the reaction vessel 22 or transfer tube 79). In other aspects, the plasma separation device 18 is integrated with the reaction vessel 22 or transfer tube 79. For example, in some aspects, the plasma separation device 18 is integrated within a housing 54 of the reaction vessel 22 (i.e., the plasma separation device 18 is not intended to be removed from the reaction vessel 22 or transfer tube 79 by a user).

The plasma separation device 18 includes a filter, a membrane, a synthetic paper, or any combinations thereof. In one aspect, the plasma separation device 18 is removably coupled to the reaction vessel 22 or transfer tube 79 with a removable coupling 58. In some aspects, the removable coupling 58 includes a threaded configuration. In other embodiments, the removable coupling 58 includes a retaining member positioned on the reaction vessel 22 or transfer tube 79 to hold the plasma separation device 18 in position once the plasma separation device 18 is installed on the reaction vessel 22 or transfer tube 79. The outlet 50 of the plasma separation device 18 is placed in fluid communication with an aperture 62 on the reaction vessel 22 or aperture 83 of the transfer tube 79. In other words, the processed capillary blood sample flows from the outlet 50 of the plasma separation device 18 into the aperture 62 of the reaction vessel 22 or aperture 83 of the transfer tube 79.

In still further aspects, the receptacle 38 is removably coupled to the plasma separation device 18 with a removable coupling 66 (FIG. 2C). In some aspects, the receptacle 38 is coupled to the plasma separation device 18 after the plasma separation device 18 is coupled to the reaction vessel 22 or transfer tube 79. In yet further aspects, the cap 42 is removed before coupling the receptacle 38 with the plasma separation device 18. In some aspects, the cap 42 remains in place as the receptacle 38 is coupled with the plasma separation device 18. In some aspects, the cap 42 is pierced as the receptacle 38 is coupled to the plasma separation device 18, permitting the capillary blood sample to flow into the plasma separation device 18.

In still further aspects, the plasma separation device 18 is removably coupled to the reaction vessel 22 or transfer tube 79 and the receptacle 38. With reference to FIG. 2D, the plasma separation device 18 is positioned between the receptacle 38 and the reaction vessel 22. In other aspects, the plasma separation device 18 is positioned between the receptacle 38 and the transfer tube 79. In some aspects, the receptacle 38 is squeezed by a user or operator to force the capillary blood sample through the plasma separation device 18 and into the reaction vessel 22 or transfer tube 79. In other aspects, the receptacle 38 includes a plunger to force the capillary blood sample through the plasma separation device 18 and into the reaction vessel 22 or transfer tube 79. In other embodiment, the capillary blood sample is gravity-fed through the plasma separation device 18 and into the reaction vessel 22 or transfer tube 79.

With reference to FIG. 2D, the reaction vessel 22 in some aspects is a microfluidic cartridge. The processed capillary blood sample flows from the plasma separation device 18 to the reaction vessel 22 in which assays are performed. In some aspects, the assay is for UCH-L1, GFAP, CK-MB, β-hCG, TSH, homocysteine, free T4 or any combinations thereof or a combination thereof.

In still further aspects, with reference to FIG. 3, the plasma separation device 18 is placed in fluid communication with an aperture 62 at any point along the reaction vessel 22 (e.g., microfluidic cartridge). In some aspects, the plasma separation device 18 is placed in fluid communication with the aperture 62 at one end, on the side or in the middle of the reaction vessel 22 (e.g., microfluidic cartridge). In some aspects, as shown in FIG. 4, the plasma separation device 18 is placed in fluid communication with the aperture 62 (e.g., microfluidic cartridge) at an end or side of the reaction vessel 22 at an angle, such as, for example, at about 5 degrees, about 10 degrees, about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees, or at about 90 degrees (e.g., perpendicular, which forms an L or J shape). In other aspects, as shown in FIG. 5, the plasma separation device 18 is placed in fluid communication with at least one aperture 62 at one end or side of the reaction vessel 22 (e.g., microfluidic cartridge).

In one aspect, with reference to FIG. 2E, with the processed capillary blood sample loaded into the reaction vessel 22, the reaction vessel 22 is inserted into the instrument 26. In the illustrated embodiment, the reaction vessel 22 is inserted into a bottom portion 70 of a handle 74. The instrument 26 includes a display 78 configured to communicate the result of UCH-L1, GFAP, CK-MB, β-hCG, TSH, homocysteine, free T4 or any combinations thereof determined in the sample. For example, a display 78 on an instrument 26 may display the result as indicating that the amount of UCH-L1, GFAP, CK-MB, β-hCG, TSH, homocysteine, free T4 or any combinations thereof in the subject is elevated, not elevated or that the test should be repeated. In some aspects, the result is provided with visual, audio, or haptic feedback.

In another aspect, with reference to FIG. 6B, the processed capillary blood sample in the transfer tube 79 is loaded into the instrument 81. Once loaded, a portion of the processed capillary blood sample is removed from transfer tube 79 and placed into a reaction vessel (e.g., a tube) which is already contained in instrument 81, to carry out the assay for UCH-L1, GFAP, CK-MB, β-hCG, TSH, homocysteine, free T4 or any combinations thereof. The portion of the processed capillary blood sample can be removed from the transfer tube 79 to the reaction vessel using a mechanical pipetting system or other techniques known in the art. The instrument 81 includes a display 82 configured to communicate the result of UCH-L1, GFAP, CK-MB, β-hCG, TSH, homocysteine, free T4 or any combinations thereof determined in the sample. For example, a display 82 on an instrument 81 may display the result as indicating that the amount of UCH-L1, GFAP, CK-MB, β-hCG, TSH, homocysteine, free T4 or any combinations thereof in the subject is elevated, not elevated or that the test should be repeated. In some aspects, the result is provided with visual, audio, or haptic feedback.

In some aspects, the result is communicated in about 4 minutes from the time the sample is collected (e.g., from the time of an injury or suspected injury). In some aspects, the result is communicated in about 10 minutes, about 15 minutes, about 20 minutes, about 25 minutes, about 30 minutes, about 35 minutes, or about 40 minutes from the time the sample is collected (e.g., from the time of an injury or suspected injury). In some aspects, the result is communicated within a range of about 4 minutes to about 40 minutes from the time the sample is collected (such as, for example, from the time of injury or suspected injury). In some aspects, the result is communicated within a range of about 4 minutes to about 30 minutes from the time the sample is collected (such as, for example, from the time of injury or suspected injury). In some aspects, the result is communicated within a range of about 4 minutes to about 20 minutes from the time the sample is collected (such as, for example, from the time of injury or suspected injury).

Advantageously, a portion of the system 10 is usable in a decentralized setting. In other words, the system 10 is portable. Advantageously, the system 10 or parts thereof are reusable for a plurality of samples and/or subjects. In some embodiments, the microsampling device 14, the plasma separation device 18 and the reaction vessel 22 or transfer tube 79 are single-use components (i.e., usable for a single test on a single subject); while the instruments 26 and 81 are reusable to analyze a plurality of reaction vessels 22.

### 6. Treatment and Monitoring of Subjects Who Have Sustained an Injury to the Head

The subject identified in the methods described above may be treated or monitored. In some embodiments, the method further includes treating the subject, such as a human subject, with a traumatic brain injury treatment, such as any treatments known in the art. For example, treatment of traumatic brain injury can take a variety of forms depending on the severity of the injury to the head. For example, for subjects suffering from mild TBI, the treatment may include one or more of rest, abstaining from physical activities, such as sports, avoiding light or wearing sunglasses when out in the light, medication for relief of a headache or migraine, anti-nausea medication, etc. Treatment for patients suffering from moderate, severe or moderate to severe TBI might include administration of one or more appropriate medications (such as, for example, diuretics, anti-convulsant medications, medications to sedate and put an individual in a drug-induced coma, or other pharmaceutical or biopharmaceutical medications (either known or developed in the future for treatment of TBI), one or more surgical procedures (such as, for example, removal of a hematoma, repairing a skull fracture, decompressive craniectomy, etc.), protecting the airway, and one or more therapies (such as, for example one or more rehabilitation, cognitive behavioral therapy, anger management, counseling psychology, etc.). In some embodiments, the method further includes monitoring the subject, such as a human subject. In some embodiments, a subject may be monitored with CT scan or MRI procedure.

### 7. Methods for Measuring the Level of UCH-L1

In the methods described above, UCH-L1 levels can be measured by any means, such as antibody dependent methods, such as immunoassays, protein immunoprecipitation, immunoelectrophoresis, chemical analysis, SDS-PAGE and Western blot analysis, protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Also, the assay can be employed in clinical chemistry format such as would be known by one skilled in the art.

In some embodiments, measuring the level of UCH-L1 includes contacting the sample with a first specific binding member and second specific binding member. In some embodiments the first specific binding member is a capture antibody and the second specific binding member is a detection antibody. In some embodiments, measuring the level of UCH-L1 includes contacting the sample, either simultaneously or sequentially, in any order: (1) a capture antibody (e.g., UCH-L1-capture antibody), which binds to an epitope on UCH-L1 or UCH-L1 fragment to form a capture antibody-UCH-L1 antigen complex (e.g., UCH-L1-capture antibody-UCH-L1 antigen complex), and (2) a detection antibody (e.g., UCH-L1-detection antibody), which includes a detectable label and binds to an epitope on UCH-L1 that is not bound by the capture antibody, to form a UCH-L1 antigen-detection antibody complex (e.g., UCH-L1 antigen-UCH-L1-detection antibody complex), such that a capture antibody-UCH-L1 antigen-detection antibody complex (e.g., UCH-L1-capture antibody-UCH-L1 antigen-UCH-L1-detection antibody complex) is formed, and measuring the amount or concentration of UCH-L1 in the sample based on the signal generated by the detectable label in the capture antibody-UCH-L1 antigen-detection antibody complex.

In some embodiments, the first specific binding member is immobilized on a solid support. In some embodiments, the second specific binding member is immobilized on a solid support. In some embodiments, the first specific binding member is a UCH-L1 antibody as described below.

In some embodiments, the sample is diluted or undiluted. The sample can be from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, about 15 microliters or about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters or about 25 microliters. In some embodiments, the sample is from about 1 to about 150 microliters or less or from about 1 to about 25 microliters or less.

Some instruments (such as, for example the Abbott Laboratories instrument ARCHITECT®, and other core laboratory instruments) other than a point-of-care device may be capable of measuring levels of UCH-L1 in a sample higher or greater than 25,000 μg/mL.

Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety

### 8. UCH-L1 Antibodies

The methods described herein may use an isolated antibody that specifically binds to ubiquitin carboxy-terminal hydrolase L1 (“UCH-L1”) (or fragments thereof), referred to as “UCH-L1 antibody.” The UCH-L1 antibodies can be used to assess the UCH-L1 status as a measure of traumatic brain injury, detect the presence of UCH-L1 in a sample, quantify the amount of UCH-L1 present in a sample, or detect the presence of and quantify the amount of UCH-L1 in a sample.

a. Ubiquitin Carboxy-Terminal Hydrolase L1 (UCH-L1)

Ubiquitin carboxy-terminal hydrolase L1 (“UCH-L1”), which is also known as “ubiquitin C-terminal hydrolase,” is a deubiquitinating enzyme. UCH-L1 is a member of a gene family whose products hydrolyze small C-terminal adducts of ubiquitin to generate the ubiquitin monomer. Expression of UCH-L1 is highly specific to neurons and to cells of the diffuse neuroendocrine system and their tumors. It is abundantly present in all neurons (accounts for 1-2% of total brain protein), expressed specifically in neurons and testis/ovary. The catalytic triad of UCH-L1 contains a cysteine at position 90, an aspartate at position 176, and a histidine at position 161 that are responsible for its hydrolase activity.

Human UCH-L1 may have the following amino acid sequence:

(SEQ ID NO: 1)
MQLKPMEINPEMLNKVLSRLGVAGQWRFVDVLGLEEESLGSVPAP
ACALLLLFPLTAQHENFRKKQIEELKGQEVSPKVYFMKQTIGNSC
GTIGLIHAVANNQDKLGFEDGSVLKQFLSETEKMSPEDRAKCFEK
NEAIQAAHDAVAQEGQCRVDDKVNFHFILFNNVDGHLYELDGRMP
FPVNHGASSEDTLLKDAAKVCREFTEREQGEVRESAVALCKAA.

The human UCH-L1 may be a fragment or variant of SEQ ID NO: 1. The fragment of UCH-L1 may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise a contiguous number of amino acids from SEQ ID NO: 1.

b. UCH-L1-Recognizing Antibody

The antibody is an antibody that binds to UCH-L1, a fragment thereof, an epitope of UCH-L1, or a variant thereof. The antibody may be a fragment of the anti-UCH-L1 antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

The anti-UCH-L1 antibodies may be a chimeric anti-UCH-L1 or humanized anti-UCH-L1 antibody. In one embodiment, both the humanized antibody and chimeric antibody are monovalent. In one embodiment, both the humanized antibody and chimeric antibody comprise a single Fab region linked to an Fc region.

Human antibodies may be derived from phage-display technology or from transgenic mice that express human immunoglobulin genes. The human antibody may be generated as a result of a human in vivo immune response and isolated. See, for example, Funaro et al., BMC Biotechnology, 2008 (8): 85. Therefore, the antibody may be a product of the human and not animal repertoire. Because it is of human origin, the risks of reactivity against self-antigens may be minimized. Alternatively, standard yeast display libraries and display technologies may be used to select and isolate human anti-UCH-L1 antibodies. For example, libraries of naïve human single chain variable fragments (scFv) may be used to select human anti-UCH-L1 antibodies. Transgenic animals may be used to express human antibodies.

Humanized antibodies may be antibody molecules from non-human species antibody that binds the desired antigen having one or more complementarity determining regions (CDRs) from the non-human species and framework regions from a human immunoglobulin molecule.

The antibody is distinguishable from known antibodies in that it possesses different biological function(s) than those known in the art.

### (1) Epitope

The antibody may immunospecifically bind to UCH-L1 (SEQ ID NO: 1), a fragment thereof, or a variant thereof. The antibody may immunospecifically recognize and bind at least three amino acids, at least four amino acids, at least five amino acids, at least six amino acids, at least seven amino acids, at least eight amino acids, at least nine amino acids, or at least ten amino acids within an epitope region. The antibody may immunospecifically recognize and bind to an epitope that has at least three contiguous amino acids, at least four contiguous amino acids, at least five contiguous amino acids, at least six contiguous amino acids, at least seven contiguous amino acids, at least eight contiguous amino acids, at least nine contiguous amino acids, or at least ten contiguous amino acids of an epitope region.

c. Antibody Preparation/Production

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77:4216-4220 (1980)), used with a DHFR selectable marker, e.g., as described in Kaufman and Sharp, J. Mol. Biol., 159:601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., binds human UCH-L1) and the other heavy and light chain are specific for an antigen other than human UCH-L1 by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with UCH-L1 or a fragment and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH::VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass).; Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994)).

An affinity matured antibody may be produced by any one of a number of procedures that are known in the art. For example, see Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); Hawkins et al, J. Mol. Biol., 226:889-896 (1992). Selective mutation at selective mutagenesis positions and at contact or hypermutation positions with an activity enhancing amino acid residue is described in U.S. Pat. No. 6,914,128 B1.

Antibody variants can also be prepared using delivering a polynucleotide encoding an antibody to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbiol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFvs), including tobacco seeds and potato tubers. See, e.g., Conrad et al. (1998) Plant Mol. Biol. 38:101-109 and reference cited therein. Thus, antibodies can also be produced using transgenic plants, according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al., (1995) Protein Eng. 8 (10): 1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (131I), yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

Antibody production via the use of hybridoma technology, the selected lymphocyte antibody method (SLAM), transgenic animals, and recombinant antibody libraries is described in more detail below.

### (1) Anti-UCH-L1 Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody of the disclosure wherein, preferably, the hybridoma is generated by fusing splenocytes isolated from an animal, e.g., a rat or a mouse, immunized with UCH-L1 with myeloma cells and then screening the hybridomas resulting from the fusion for hybridoma clones that secrete an antibody able to bind a polypeptide of the disclosure. Briefly, rats can be immunized with a UCH-L1 antigen. In a preferred embodiment, the UCH-L1 antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with a UCH-L1 antigen, antibodies and/or antibody-producing cells may be obtained from the animal. An anti-UCH-L1 antibody-containing serum is obtained from the animal by bleeding or sacrificing the animal. The serum may be used as it is obtained from the animal, an immunoglobulin fraction may be obtained from the serum, or the anti-UCH-L1 antibodies may be purified from the serum. Serum or immunoglobulins obtained in this manner are polyclonal, thus having a heterogeneous array of properties.

Once an immune response is detected, e.g., antibodies specific for the antigen UCH-L1 are detected in the rat serum, the rat spleen is harvested and splenocytes isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding UCH-L1. Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed, and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using UCH-L1, or a portion thereof, or a cell expressing UCH-L1. In a preferred embodiment, the initial screening is performed using an enzyme-linked immunosorbent assay (ELISA) or a radioimmunoassay (RIA), preferably an ELISA. An example of ELISA screening is provided in PCT Publication No. WO 00/37504.

Anti-UCH-L1 antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another preferred embodiment, the hybridomas are human hybridomas, in which a human non-secretory myeloma is fused with a human cell expressing an anti-UCH-L1 antibody.

Antibody fragments that recognize specific epitopes may be generated by known techniques. For example, Fab and F(ab′)₂ fragments of the disclosure may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CHI domains of the heavy chains, and a disulfide-forming hinge region of the parent IgG molecule. Accordingly, an F(ab′)₂ fragment is still capable of crosslinking antigen molecules like the parent IgG molecule.

### (2) Anti-UCH-L1 Monoclonal Antibodies Using SLAM

In another aspect of the disclosure, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93:7843-7848 (1996). In this method, single cells secreting antibodies of interest, e.g., lymphocytes derived from any one of the immunized animals are screened using an antigen-specific hemolytic plaque assay, wherein the antigen UCH-L1, a subunit of UCH-L1, or a fragment thereof, is coupled to sheep red blood cells using a linker, such as biotin, and used to identify single cells that secrete antibodies with specificity for UCH-L1. Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to UCH-L1. The amplified immunoglobulin sequences further can be manipulated in vitro, such as by in vitro affinity maturation method. See, for example, PCT Publication No. WO 97/29131 and PCT Publication No. WO 00/56772.

### (3) Anti-UCH-L1 Monoclonal Antibodies Using Transgenic Animals

In another embodiment of the disclosure, antibodies are produced by immunizing a non-human animal comprising some, or all, of the human immunoglobulin locus with a UCH-L1 antigen. In an embodiment, the non-human animal is a XENOMOUSE® transgenic mouse, an engineered mouse strain that comprises large fragments of the human immunoglobulin loci and is deficient in mouse antibody production. See, e.g., Green et al., Nature Genetics, 7:13-21 (1994) and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al., Nature Genetics, 15:146-156 (1997), Green and Jakobovits, J. Exp. Med., 188:483-495 (1998), the disclosures of which are hereby incorporated by reference.

### (4) Anti-UCH-L1 Monoclonal Antibodies Using Recombinant Antibody Libraries

In vitro methods also can be used to make the antibodies of the disclosure, wherein an antibody library is screened to identify an antibody having the desired UCH-L1-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9:1369-1372 (1991); Hay et al., Hum. Antibod. Hybridomas, 3:81-85 (1992); Huse et al., Science, 246:1275-1281 (1989); McCafferty et al., Nature, 348:552-554 (1990); Griffiths et al., EMBO J., 12:725-734 (1993); Hawkins et al., J. Mol. Biol., 226:889-896 (1992); Clackson et al., Nature, 352:624-628 (1991); Gram et al., Proc. Natl. Acad. Sci. USA, 89:3576-3580 (1992); Garrard et al., Bio/Technology, 9:1373-1377 (1991); Hoogenboom et al., Nucl. Acids Res., 19:4133-4137 (1991); Barbas et al., Proc. Natl. Acad. Sci. USA, 88:7978-7982 (1991); U.S. Patent Application Publication No. 2003/0186374; and PCT Publication No. WO 97/29131, the contents of each of which are incorporated herein by reference.

The recombinant antibody library may be from a subject immunized with UCH-L1, or a portion of UCH-L1. Alternatively, the recombinant antibody library may be from a naive subject, i.e., one who has not been immunized with UCH-L1, such as a human antibody library from a human subject who has not been immunized with human UCH-L1. Antibodies of the disclosure are selected by screening the recombinant antibody library with the peptide comprising human UCH-L1 to thereby select those antibodies that recognize UCH-L1. Methods for conducting such screening and selection are well known in the art, such as described in the references in the preceding paragraph. To select antibodies of the disclosure having particular binding affinities for UCH-L1, such as those that dissociate from human UCH-L1 with a particular Koff rate constant, the art-known method of surface plasmon resonance can be used to select antibodies having the desired K_{off} rate constant. To select antibodies of the disclosure having a particular neutralizing activity for hUCH-L1, such as those with a particular IC₅₀, standard methods known in the art for assessing the inhibition of UCH-L1 activity may be used.

In one aspect, the disclosure pertains to an isolated antibody, or an antigen-binding portion thereof, that binds human UCH-L1. Preferably, the antibody is a neutralizing antibody. In various embodiments, the antibody is a recombinant antibody or a monoclonal antibody.

For example, antibodies can also be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used to make the antibodies include those disclosed in Brinkmann et al., J. Immunol. Methods, 182:41-50 (1995); Ames et al., J. Immunol. Methods, 184:177-186 (1995); Kettleborough et al., Eur. J. Immunol., 24:952-958 (1994); Persic et al., Gene, 187:9-18 (1997); Burton et al., Advances in Immunology, 57:191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments can also be employed using methods known in the art such as those disclosed in PCT publication No. WO 92/22324; Mullinax et al., BioTechniques, 12 (6): 864-869 (1992); Sawai et al., Am. J. Reprod. Immunol., 34:26-34 (1995); and Better et al., Science, 240:1041-1043 (1988). Examples of techniques which can be used to produce single-chain Fvs and antibodies include those described in U.S. Pat. Nos. 4,946,778 and 5,258,498; Huston et al., Methods in Enzymology, 203:46-88 (1991); Shu et al., Proc. Natl. Acad. Sci. USA, 90:7995-7999 (1993); and Skerra et al., Science, 240:1038-1041 (1988).

Alternative to screening of recombinant antibody libraries by phage display, other methodologies known in the art for screening large combinatorial libraries can be applied to the identification of antibodies of the disclosure. One type of alternative expression system is one in which the recombinant antibody library is expressed as RNA-protein fusions, as described in PCT Publication No. WO 98/31700 (Szostak and Roberts), and in Roberts and Szostak, Proc. Natl. Acad. Sci. USA, 94:12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies can also be generated using yeast display methods known in the art. In yeast display methods, genetic methods are used to tether antibody domains to the yeast cell wall and display them on the surface of yeast. Such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

d. Production of Recombinant UCH-L1 Antibodies

Antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the disclosure in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies of the disclosure include Chinese Hamster Ovary (CHO cells) (including dhfr-CHO cells, described in Urlaub and Chasin, Proc. Natl. Acad. Sci. USA, 77:4216-4220 (1980), used with a DHFR selectable marker, e.g., as described in Kaufman and Sharp, J. Mol. Biol., 159:601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody of this disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the disclosure. In addition, bifunctional antibodies may be produced in which one heavy and one light chain are an antibody of the disclosure (i.e., binds human UCH-L1) and the other heavy and light chain are specific for an antigen other than human UCH-L1 by crosslinking an antibody of the disclosure to a second antibody by standard chemical crosslinking methods.

In a preferred system for recombinant expression of an antibody, or antigen-binding portion thereof, of the disclosure, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the disclosure provides a method of synthesizing a recombinant antibody of the disclosure by culturing a host cell of the disclosure in a suitable culture medium until a recombinant antibody of the disclosure is synthesized. The method can further comprise isolating the recombinant antibody from the culture medium.

### (1) Humanized Antibody

The humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one aspect, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present disclosure can be performed using any known method, such as but not limited to those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for UCH-L1 and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristics, such as increased affinity for UCH-L1, is achieved. In general, the hypervariable region residues may be directly and most substantially involved in influencing antigen binding.

As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g., mice that are capable, upon immunization, of producing a full repertoire of human antibodies in the absence of endogenous immunoglobulin production. For example, the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

e. Anti-UCH-L1 antibodies

Anti-UCH-L1 antibodies may be generated using the techniques described above as well as using routine techniques known in the art. In some embodiments, the anti-UCH-L1 antibody may be an unconjugated UCH-L1 antibody, such as UCH-L1 antibodies available from United State Biological (Catalog Number: 031320), Cell Signaling Technology (Catalog Number: 3524), Sigma-Aldrich (Catalog Number: HPA005993), Santa Cruz Biotechnology, Inc. (Catalog Numbers: sc-58593 or sc-58594), R&D Systems (Catalog Number: MAB6007), Novus Biologicals (Catalog Number: NB600-1160), Biorbyt (Catalog Number: orb33715), Enzo Life Sciences, Inc. (Catalog Number: ADI-905-520-1), Bio-Rad (Catalog Number: VMA00004), Bio Vision (Catalog Number: 6130-50), Abcam (Catalog Numbers: ab75275 or ab104938), Invitrogen Antibodies (Catalog Numbers: 480012), ThermoFisher Scientific (Catalog Numbers: MA1-46079, MA5-17235, MA1-90008, or MAI-83428), EMD Millipore (Catalog Number: MABN48), or Sino Biological Inc. (Catalog Number: 50690-R011). The anti-UCH-L1 antibody may be conjugated to a fluorophore, such as conjugated UCH-L1 antibodies available from BioVision (Catalog Number: 6960-25) or Aviva Systems Biology (Cat. Nos. OAAF01904-FITC).

### 9. Methods for Measuring the Level of GFAP

In the methods described above, GFAP levels can be measured by any means, such as antibody dependent methods, such as immunoassays, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Also, the assay can be employed in clinical chemistry format such as would be known by one skilled in the art.

In some embodiments, measuring the level of GFAP includes contacting the sample with a first specific binding member and second specific binding member. In some embodiments the first specific binding member is a capture antibody and the second specific binding member is a detection antibody. In some embodiments, measuring the level of GFAP includes contacting the sample, either simultaneously or sequentially, in any order: (1) a capture antibody (e.g., GFAP-capture antibody), which binds to an epitope on GFAP or GFAP fragment to form a capture antibody-GFAP antigen complex (e.g., GFAP-capture antibody-GFAP antigen complex), and (2) a detection antibody (e.g., GFAP-detection antibody), which includes a detectable label and binds to an epitope on GFAP that is not bound by the capture antibody, to form a GFAP antigen-detection antibody complex (e.g., GFAP antigen-GFAP-detection antibody complex), such that a capture antibody-GFAP antigen-detection antibody complex (e.g., GFAP-capture antibody-GFAP antigen-GFAP-detection antibody complex) is formed, and measuring the amount or concentration of GFAP in the sample based on the signal generated by the detectable label in the capture antibody-GFAP antigen-detection antibody complex.

In some embodiments, the first specific binding member is immobilized on a solid support. In some embodiments, the second specific binding member is immobilized on a solid support. In some embodiments, the first specific binding member is a GFAP antibody as described below.

In some embodiments, the sample is diluted or undiluted. The sample can be from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, about 15 microliters or about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters or about 25 microliters. In some embodiments, the sample is from about 1 to about 150 microliters or less or from about 1 to about 25 microliters or less.

Some instruments (such as, for example the Abbott Laboratories instrument ARCHITECT®, and other core laboratory instruments) other than a point-of-care device may be capable of measuring levels of GFAP in a sample higher or greater than 25,000 μg/mL.

Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety

### 10. GFAP Antibodies

The methods described herein may use an isolated antibody that specifically binds to Glial fibrillary acidic protein (“GFAP”) (or fragments thereof), referred to as “GFAP antibody.” The GFAP antibodies can be used to assess the GFAP status as a measure of traumatic brain injury, detect the presence of GFAP in a sample, quantify the amount of GFAP present in a sample, or detect the presence of and quantify the amount of GFAP in a sample.

a. Glial fibrillary acidic protein (GFAP)

Glial fibrillary acidic protein (GFAP) is a 50 kDa intracytoplasmic filamentous protein that constitutes a portion of the cytoskeleton in astrocytes, and it has proved to be the most specific marker for cells of astrocytic origin. GFAP protein is encoded by the GFAP gene in humans. GFAP is the principal intermediate filament of mature astrocytes. In the central rod domain of the molecule, GFAP shares considerable structural homology with the other intermediate filaments. GFAP is involved in astrocyte motility and shape by providing structural stability to astrocytic processes. Glial fibrillary acidic protein and its breakdown products (GFAP-BDP) are brain-specific proteins released into the blood as part of the pathophysiological response after traumatic brain injury (TBI). Following injury to the human CNS caused by trauma, genetic disorders, or chemicals, astrocytes proliferate and show extensive hypertrophy of the cell body and processes, and GFAP is markedly upregulated. In contrast, with increasing astrocyte malignancy, there is a progressive loss of GFAP production. GFAP can also be detected in Schwann cells, enteric glia cells, salivary gland neoplasms, metastasizing renal carcinomas, epiglottic cartilage, pituicytes, immature oligodendrocytes, papillary meningiomas, and myoepithelial cells of the breast.

Human GFAP may have the following amino acid sequence:

(SEQ ID NO: 2)
MERRRITSAARRSYVSSGEMMVGGLAPGRRLGPGTRLSLARMPPP
LPTRVDFSLAGALNAGFKETRASERAEMMELNDRFASYIEKVRFL
EQQNKALAAELNQLRAKEPTKLADVYQAELRELRLRLDQLTANSA
RLEVERDNLAQDLATVRQKLQDETNLRLEAENNLAAYRQEADEAT
LARLDLERKIESLEEEIRFLRKIHEEEVRELQEQLARQQVHVELD
VAKPDLTAALKEIRTQYEAMASSNMHEAEEWYRSKFADLTDAAAR
NAELLRQAKHEANDYRRQLQSLTCDLESLRGTNESLERQMREQEE
RHVREAASYQEALARLEEEGQSLKDEMARHLQEYQDLLNVKLALD
IEIATYRKLLEGEENRITIPVQTFSNLQIRETSLDTKSVSEGHLK
RNIVVKTVEMRDGEVIKESKQEHKDVM.

The human GFAP may be a fragment or variant of SEQ ID NO: 2. The fragment of GFAP may be between 5 and 400 amino acids, between 10 and 400 amino acids, between 50 and 400 amino acids, between 60 and 400 amino acids, between 65 and 400 amino acids, between 100 and 400 amino acids, between 150 and 400 amino acids, between 100 and 300 amino acids, or between 200 and 300 amino acids in length. The fragment may comprise a contiguous number of amino acids from SEQ ID NO: 2. The human GFAP fragment or variant of SEQ ID NO: 2 may be a GFAP breakdown product (BDP). The GFAP BDP may be 38 kDa, 42 kDa (fainter 41 kDa), 47 kDa (fainter 45 kDa); 25 kDa (fainter 23 kDa); 19 kDa, or 20 kDa.

b. GFAP-Recognizing Antibody

The antibody is an antibody that binds to GFAP, a fragment thereof, an epitope of GFAP, or a variant thereof. The antibody may be a fragment of the anti-GFAP antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

The anti-GFAP antibodies may be a chimeric anti-GFAP or humanized anti-GFAP antibody. In one embodiment, both the humanized antibody and chimeric antibody are monovalent. In one embodiment, both the humanized antibody and chimeric antibody comprise a single Fab region linked to an Fc region.

Human antibodies may be derived from phage-display technology or from transgenic mice that express human immunoglobulin genes. The human antibody may be generated as a result of a human in vivo immune response and isolated. See, for example, Funaro et al., BMC Biotechnology, 2008 (8): 85. Therefore, the antibody may be a product of the human and not animal repertoire. Because it is of human origin, the risks of reactivity against self-antigens may be minimized. Alternatively, standard yeast display libraries and display technologies may be used to select and isolate human anti-GFAP antibodies. For example, libraries of naïve human single chain variable fragments (scFv) may be used to select human anti-GFAP antibodies. Transgenic animals may be used to express human antibodies.

Humanized antibodies may be antibody molecules from non-human species antibody that binds the desired antigen having one or more complementarity determining regions (CDRs) from the non-human species and framework regions from a human immunoglobulin molecule.

The antibody is distinguishable from known antibodies in that it possesses different biological function(s) than those known in the art.

### (1) Epitope

The antibody may immunospecifically bind to GFAP (SEQ ID NO: 2), a fragment thereof, or a variant thereof. The antibody may immunospecifically recognize and bind at least three amino acids, at least four amino acids, at least five amino acids, at least six amino acids, at least seven amino acids, at least eight amino acids, at least nine amino acids, or at least ten amino acids within an epitope region. The antibody may immunospecifically recognize and bind to an epitope that has at least three contiguous amino acids, at least four contiguous amino acids, at least five contiguous amino acids, at least six contiguous amino acids, at least seven contiguous amino acids, at least eight contiguous amino acids, at least nine contiguous amino acids, or at least ten contiguous amino acids of an epitope region.

c. Antibody Preparation/Production

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, in order to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77:4216-4220 (1980)), used with a DHFR selectable marker, e.g., as described in Kaufman and Sharp, J. Mol. Biol., 159:601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., binds human GFAP) and the other heavy and light chain are specific for an antigen other than human GFAP by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with GFAP or a fragment and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH: VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass).; Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994)).

An affinity matured antibody may be produced by any one of a number of procedures that are known in the art. For example, see Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); Hawkins et al, J. Mol. Biol., 226:889-896 (1992). Selective mutation at selective mutagenesis positions and at contact or hypermutation positions with an activity enhancing amino acid residue is described in U.S. Pat. No. 6,914,128 B1.

Antibody variants can also be prepared using delivering a polynucleotide encoding an antibody to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbiol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFvs), including tobacco seeds and potato tubers. See, e.g., Conrad et al. (1998) Plant Mol. Biol. 38:101-109 and reference cited therein. Thus, antibodies can also be produced using transgenic plants, according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al. (1995) Protein Eng. 8 (10): 1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (131I), yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

Antibody production via the use of hybridoma technology, the selected lymphocyte antibody method (SLAM), transgenic animals, and recombinant antibody libraries is described in more detail below.

### (1) Anti-GFAP Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody of the disclosure wherein, preferably, the hybridoma is generated by fusing splenocytes isolated from an animal, e.g., a rat or a mouse, immunized with GFAP with myeloma cells and then screening the hybridomas resulting from the fusion for hybridoma clones that secrete an antibody able to bind a polypeptide of the disclosure. Briefly, rats can be immunized with a GFAP antigen. In a preferred embodiment, the GFAP antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with a GFAP antigen, antibodies and/or antibody-producing cells may be obtained from the animal. An anti-GFAP antibody-containing serum is obtained from the animal by bleeding or sacrificing the animal. The serum may be used as it is obtained from the animal, an immunoglobulin fraction may be obtained from the serum, or the anti-GFAP antibodies may be purified from the serum. Serum or immunoglobulins obtained in this manner are polyclonal, thus having a heterogeneous array of properties.

Once an immune response is detected, e.g., antibodies specific for the antigen GFAP are detected in the rat serum, the rat spleen is harvested and splenocytes isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding GFAP. Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed, and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using GFAP, or a portion thereof, or a cell expressing GFAP. In a preferred embodiment, the initial screening is performed using an enzyme-linked immunosorbent assay (ELISA) or a radioimmunoassay (RIA), preferably an ELISA. An example of ELISA screening is provided in PCT Publication No. WO 00/37504.

Anti-GFAP antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another preferred embodiment, the hybridomas are human hybridomas, in which a human non-secretory myeloma is fused with a human cell expressing an anti-GFAP antibody.

Antibody fragments that recognize specific epitopes may be generated by known techniques. For example, Fab and F(ab′)₂ fragments of the disclosure may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CHI domains of the heavy chains, and a disulfide-forming hinge region of the parent IgG molecule. Accordingly, an F(ab′)₂ fragment is still capable of crosslinking antigen molecules like the parent IgG molecule.

### (2) Anti-GFAP Monoclonal Antibodies Using SLAM

In another aspect of the disclosure, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93:7843-7848 (1996). In this method, single cells secreting antibodies of interest, e.g., lymphocytes derived from any one of the immunized animals are screened using an antigen-specific hemolytic plaque assay, wherein the antigen GFAP, a subunit of GFAP, or a fragment thereof, is coupled to sheep red blood cells using a linker, such as biotin, and used to identify single cells that secrete antibodies with specificity for GFAP. Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to GFAP. The amplified immunoglobulin sequences further can be manipulated in vitro, such as by in vitro affinity maturation method. See, for example, PCT Publication No. WO 97/29131 and PCT Publication No. WO 00/56772.

### (3) Anti-GFAP Monoclonal Antibodies Using Transgenic Animals

In another embodiment of the disclosure, antibodies are produced by immunizing a non-human animal comprising some, or all, of the human immunoglobulin locus with a GFAP antigen. In an embodiment, the non-human animal is a XENOMOUSE® transgenic mouse, an engineered mouse strain that comprises large fragments of the human immunoglobulin loci and is deficient in mouse antibody production. See, e.g., Green et al., Nature Genetics, 7:13-21 (1994) and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies, and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al., Nature Genetics, 15:146-156 (1997), Green and Jakobovits, J. Exp. Med., 188:483-495 (1998), the disclosures of which are hereby incorporated by reference.

### (4) Anti-GFAP Monoclonal Antibodies Using Recombinant Antibody Libraries

In vitro methods also can be used to make the antibodies of the disclosure, wherein an antibody library is screened to identify an antibody having the desired GFAP-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9:1369-1372 (1991); Hay et al., Hum. Antibod. Hybridomas, 3:81-85 (1992); Huse et al., Science, 246:1275-1281 (1989); McCafferty et al., Nature, 348:552-554 (1990); Griffiths et al., EMBO J., 12:725-734 (1993); Hawkins et al., J. Mol. Biol., 226:889-896 (1992); Clackson et al., Nature, 352:624-628 (1991); Gram et al., Proc. Natl. Acad. Sci. USA, 89:3576-3580 (1992); Garrard et al., Bio/Technology, 9:1373-1377 (1991); Hoogenboom et al., Nucl. Acids Res., 19:4133-4137 (1991); Barbas et al., Proc. Natl. Acad. Sci. USA, 88:7978-7982 (1991); U.S. Patent Application Publication No. 2003/0186374; and PCT Publication No. WO 97/29131, the contents of each of which are incorporated herein by reference.

The recombinant antibody library may be from a subject immunized with GFAP, or a portion of GFAP. Alternatively, the recombinant antibody library may be from a naive subject, i.e., one who has not been immunized with GFAP, such as a human antibody library from a human subject who has not been immunized with human GFAP. Antibodies of the disclosure are selected by screening the recombinant antibody library with the peptide comprising human GFAP to thereby select those antibodies that recognize GFAP. Methods for conducting such screening and selection are well known in the art, such as described in the references in the preceding paragraph. To select antibodies of the disclosure having particular binding affinities for GFAP, such as those that dissociate from human GFAP with a particular K_{off} rate constant, the art-known method of surface plasmon resonance can be used to select antibodies having the desired K_{off} rate constant. To select antibodies of the disclosure having a particular neutralizing activity for GFAP, such as those with a particular IC₅₀, standard methods known in the art for assessing the inhibition of GFAP activity may be used.

In one aspect, the disclosure pertains to an isolated antibody, or an antigen-binding portion thereof, that binds human GFAP. Preferably, the antibody is a neutralizing antibody. In various embodiments, the antibody is a recombinant antibody or a monoclonal antibody.

For example, antibodies can also be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used to make the antibodies include those disclosed in Brinkmann et al., J. Immunol. Methods, 182:41-50 (1995); Ames et al., J. Immunol. Methods, 184:177-186 (1995); Kettleborough et al., Eur. J. Immunol., 24:952-958 (1994); Persic et al., Gene, 187:9-18 (1997); Burton et al., Advances in Immunology, 57:191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments can also be employed using methods known in the art such as those disclosed in PCT publication No. WO 92/22324; Mullinax et al., BioTechniques, 12 (6): 864-869 (1992); Sawai et al., Am. J. Reprod. Immunol., 34:26-34 (1995); and Better et al., Science, 240:1041-1043 (1988). Examples of techniques which can be used to produce single-chain Fvs and antibodies include those described in U.S. Pat. Nos. 4,946,778 and 5,258,498; Huston et al., Methods in Enzymology, 203:46-88 (1991); Shu et al., Proc. Natl. Acad. Sci. USA, 90:7995-7999 (1993); and Skerra et al., Science, 240:1038-1041 (1988).

Alternative to screening of recombinant antibody libraries by phage display, other methodologies known in the art for screening large combinatorial libraries can be applied to the identification of antibodies of the disclosure. One type of alternative expression system is one in which the recombinant antibody library is expressed as RNA-protein fusions, as described in PCT Publication No. WO 98/31700 (Szostak and Roberts), and in Roberts and Szostak, Proc. Natl. Acad. Sci. USA, 94:12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies can also be generated using yeast display methods known in the art. In yeast display methods, genetic methods are used to tether antibody domains to the yeast cell wall and display them on the surface of yeast. Such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

d. Production of Recombinant GFAP Antibodies

Antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the disclosure in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies of the disclosure include Chinese Hamster Ovary (CHO cells) (including dhfr-CHO cells, described in Urlaub and Chasin, Proc. Natl. Acad. Sci. USA, 77:4216-4220 (1980), used with a DHFR selectable marker, e.g., as described in Kaufman and Sharp, J. Mol. Biol., 159:601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody of this disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the disclosure. In addition, bifunctional antibodies may be produced in which one heavy and one light chain are an antibody of the disclosure (i.e., binds human GFAP) and the other heavy and light chain are specific for an antigen other than human GFAP by crosslinking an antibody of the disclosure to a second antibody by standard chemical crosslinking methods.

In a preferred system for recombinant expression of an antibody, or antigen-binding portion thereof, of the disclosure, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the disclosure provides a method of synthesizing a recombinant antibody of the disclosure by culturing a host cell of the disclosure in a suitable culture medium until a recombinant antibody of the disclosure is synthesized. The method can further comprise isolating the recombinant antibody from the culture medium.

### (1) Humanized Antibody

The humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one aspect, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG 1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present disclosure can be performed using any known method, such as but not limited to those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for GFAP and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristics, such as increased affinity for GFAP, is achieved. In general, the hypervariable region residues may be directly and most substantially involved in influencing antigen binding.

As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g. mice that are capable, upon immunization, of producing a full repertoire of human antibodies in the absence of endogenous immunoglobulin production. For example, the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

e. Anti-GFAP antibodies

Anti-GFAP antibodies may be generated using the techniques described above as well as using routine techniques known in the art. In some embodiments, the anti-GFAP antibody may be an unconjugated GFAP antibody, such as GFAP antibodies available from Dako (Catalog Number: M0761), ThermoFisher Scientific (Catalog Numbers: MA5-12023, A-21282, 13-0300, MA1-19170, MA1-19395, MA5-15086, MA5-16367, MA1-35377, MA1-06701, or MA1-20035), AbCam (Catalog Numbers: ab10062, ab4648, ab68428, ab33922, ab207165, ab190288, ab115898, or ab21837), EMD Millipore (Catalog Numbers: FCMAB257P, MAB360, MAB3402, 04-1031, 04-1062, MAB5628), Santa Cruz (Catalog Numbers: sc-166481, sc-166458, sc-58766, sc-56395, sc-51908, sc-135921, sc-71143, sc-65343, or sc-33673), Sigma-Aldrich (Catalog Numbers: G3893 or G6171) or Sino Biological Inc. (Catalog Number: 100140-R012-50). The anti-GFAP antibody may be conjugated to a fluorophore, such as conjugated GFAP antibodies available from ThermoFisher Scientific (Catalog Numbers: A-21295 or A-21294), EMD Millipore (Catalog Numbers: MAB3402X, MAB3402B, MAB3402B, or MAB3402C3) or AbCam (Catalog Numbers: ab49874 or ab194325).

9. Methods for Measuring the Level of CK-MB, β-hCG, TSH, Homocysteine, and/or Free T4

In the methods described above, CK-MB, β-hCG, TSH, homocysteine, and/or free T4 levels can be measured by any means, such as antibody dependent methods, such as immunoassays, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Also, the assay can be employed in clinical chemistry format such as would be known by one skilled in the art.

In some embodiments, measuring the level of CK-MB, β-hCG, TSH, homocysteine, and/or free T4 includes contacting the sample with a first specific binding member and second specific binding member. In some embodiments the first specific binding member is a capture antibody and the second specific binding member is a detection antibody. In some embodiments, measuring the level of CK-MB, β-hCG, TSH, homocysteine, and/or free T4 includes contacting the sample, either simultaneously or sequentially, in any order: (1) a capture antibody (e.g., CK-MB, β-hCG, TSH, homocysteine, or free T4-capture antibody), which binds to an epitope on CK-MB, β-hCG, TSH, homocysteine, or free T4 or CK-MB, β-hCG, TSH, homocysteine, or free T4 fragment to form a capture antibody-CK-MB, β-hCG, TSH, homocysteine, or free T4 antigen complex (e.g., CK-MB, β-hCG, TSH, homocysteine, or free T4-capture antibody-CK-MB, β-hCG, TSH, homocysteine, or free T4 antigen complex), and (2) a detection antibody (e.g., CK-MB, β-hCG, TSH, homocysteine, or free T4-detection antibody), which includes a detectable label and binds to an epitope on CK-MB, β-hCG, TSH, homocysteine, or free T4 that is not bound by the capture antibody, to form a CK-MB, β-hCG, TSH, homocysteine, or free T4 antigen-detection antibody complex (e.g., CK-MB, β-hCG, TSH, homocysteine, or free T4 antigen-CK-MB, β-hCG, TSH, homocysteine, or free T4-detection antibody complex), such that a capture antibody-CK-MB, β-hCG, TSH, homocysteine, or free T4 antigen-detection antibody complex (e.g., CK-MB, β-hCG, TSH, homocysteine, or free T4 capture antibody-CK-MB, β-hCG, TSH, homocysteine, or free T4 antigen-CK-MB, β-hCG, TSH, homocysteine, or free T4-detection antibody complex) is formed, and measuring the amount or concentration of CK-MB, β-hCG, TSH, homocysteine, or free T4 in the sample based on the signal generated by the detectable label in the capture antibody-CK-MB, β-hCG, TSH, homocysteine, or free T4 antigen-detection antibody complex.

In some embodiments, the first specific binding member is immobilized on a solid support. In some embodiments, the second specific binding member is immobilized on a solid support. In some embodiments, the first specific binding member is a CK-MB, β-hCG, TSH, homocysteine, or free T4 antibody as described below.

In some embodiments, the sample is diluted or undiluted. The sample can be from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, about 15 microliters or about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters or about 25 microliters. In some embodiments, the sample is from about 1 to about 150 microliters or less or from about 1 to about 25 microliters or less.

Some instruments (such as, for example the Abbott Laboratories instrument ARCHITECT®, and other core laboratory instruments) other than a point-of-care device may be capable of measuring levels of CK-MB, β-hCG, TSH, homocysteine, or free T4 in a sample higher or greater than 25,000 μg/mL.

Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety

10. CK-MB, β-hCG, TSH, Homocysteine, and/or free T4 Antibodies

The methods described herein may use an isolated antibody that specifically binds to CK-MB, β-hCG, TSH, homocysteine, or free T4 (or fragments thereof), referred to as “CK-MB, β-hCG, TSH, homocysteine, or free T4 antibody.” The CK-MB, β-hCG, TSH, homocysteine, or free T4 antibodies can be used to assess the CK-MB, β-hCG, TSH, homocysteine, or free T4 status as a measure of traumatic brain injury, detect the presence of CK-MB, β-hCG, TSH, homocysteine, or free T4 in a sample, quantify the amount of CK-MB, β-hCG, TSH, homocysteine, or free T4 present in a sample, or detect the presence of and quantify the amount of CK-MB, β-hCG, TSH, homocysteine, or free T4 in a sample.

a. CK-MB, β-hCG, TSH, Homocysteine, or free T4-Recognizing Antibody

antibody is an antibody that binds to CK-MB, β-hCG, TSH, homocysteine, or free T4, a fragment thereof, an epitope of CK-MB, β-hCG, TSH, homocysteine, or free T4, or a variant thereof. The antibody may be a fragment of the anti-CK-MB, β-hCG, TSH, homocysteine, or free T4 antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

The anti-CK-MB, β-hCG, TSH, homocysteine, or free T4 antibodies may be a chimeric anti-CK-MB, β-hCG, TSH, homocysteine, or free T4 or humanized anti-CK-MB, β-hCG, TSH, homocysteine, or free T4 antibodies. In one embodiment, both the humanized antibody and chimeric antibody are monovalent. In one embodiment, both the humanized antibody and chimeric antibody comprise a single Fab region linked to an Fc region.

Human antibodies may be derived from phage-display technology or from transgenic mice that express human immunoglobulin genes. The human antibody may be generated as a result of a human in vivo immune response and isolated. See, for example, Funaro et al., BMC Biotechnology, 2008 (8): 85. Therefore, the antibody may be a product of the human and not animal repertoire. Because it is of human origin, the risks of reactivity against self-antigens may be minimized. Alternatively, standard yeast display libraries and display technologies may be used to select and isolate human anti-CK-MB, β-hCG, TSH, homocysteine, or free T4 antibodies. For example, libraries of naïve human single chain variable fragments (scFv) may be used to select human anti-CK-MB, β-hCG, TSH, homocysteine, or free T4 antibodies. Transgenic animals may be used to express human antibodies.

Humanized antibodies may be antibody molecules from non-human species antibody that binds the desired antigen having one or more complementarity determining regions (CDRs) from the non-human species and framework regions from a human immunoglobulin molecule.

The antibody is distinguishable from known antibodies in that it possesses different biological function(s) than those known in the art.

b. Antibody Preparation/Production

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, in order to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77:4216-4220 (1980)), used with a DHFR selectable marker, e.g., as described in Kaufman and Sharp, J. Mol. Biol., 159:601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., binds human CK-MB, β-hCG, TSH, homocysteine, or free T4) and the other heavy and light chain are specific for an antigen other than human CK-MB, β-hCG, TSH, homocysteine, or free T4 by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with CK-MB, β-hCG, TSH, homocysteine, or free T4 or a fragment and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH: VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass); Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994)).

An affinity matured antibody may be produced by any one of a number of procedures that are known in the art. For example, see Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); Hawkins et al, J. Mol. Biol., 226:889-896 (1992). Selective mutation at selective mutagenesis positions and at contact or hypermutation positions with an activity enhancing amino acid residue is described in U.S. Pat. No. 6,914,128 B1.

Antibody variants can also be prepared using delivering a polynucleotide encoding an antibody to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbiol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFvs), including tobacco seeds and potato tubers. See, e.g., Conrad et al. (1998) Plant Mol. Biol. 38:101-109 and reference cited therein. Thus, antibodies can also be produced using transgenic plants, according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al. (1995) Protein Eng. 8 (10): 1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (131I), yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

Antibody production via the use of hybridoma technology, the selected lymphocyte antibody method (SLAM), transgenic animals, and recombinant antibody libraries is described in more detail below.

(1) Anti-CK-MB, β-hCG, TSH, Homocysteine, or free T4 Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody of the disclosure wherein, preferably, the hybridoma is generated by fusing splenocytes isolated from an animal, e.g., a rat or a mouse, immunized with CK-MB, β-hCG, TSH, homocysteine, or free T4 with myeloma cells and then screening the hybridomas resulting from the fusion for hybridoma clones that secrete an antibody able to bind a polypeptide of the disclosure. Briefly, rats can be immunized with a CK-MB, β-hCG, TSH, homocysteine, or free T4 antigen. In a preferred embodiment, the CK-MB, β-hCG, TSH, homocysteine, or free T4 antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with a CK-MB, β-hCG, TSH, homocysteine, or free T4 antigen, antibodies and/or antibody-producing cells may be obtained from the animal. An anti-CK-MB, β-hCG, TSH, homocysteine, or free T4 antibody-containing serum is obtained from the animal by bleeding or sacrificing the animal. The serum may be used as it is obtained from the animal, an immunoglobulin fraction may be obtained from the serum, or the anti-CK-MB, β-hCG, TSH, homocysteine, or free T4 antibodies may be purified from the serum. Serum or immunoglobulins obtained in this manner are polyclonal, thus having a heterogeneous array of properties.

Once an immune response is detected, e.g., antibodies specific for the antigen CK-MB, β-hCG, TSH, homocysteine, or free T4 are detected in the rat serum, the rat spleen is harvested and splenocytes isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding CK-MB, β-hCG, TSH, homocysteine, or free T4. Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed, and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using CK-MB, β-hCG, TSH, homocysteine, or free T4, or a portion thereof, or a cell expressing CK-MB, β-hCG, TSH, homocysteine, or free T4. In a preferred embodiment, the initial screening is performed using an enzyme-linked immunosorbent assay (ELISA) or a radioimmunoassay (RIA), preferably an ELISA. An example of ELISA screening is provided in PCT Publication No. WO 00/37504.

Anti-CK-MB, β-hCG, TSH, homocysteine, or free T4 antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another preferred embodiment, the hybridomas are human hybridomas, in which a human non-secretory myeloma is fused with a human cell expressing an anti-CK-MB, β-hCG, TSH, homocysteine, or free T4 antibody.

Antibody fragments that recognize specific epitopes may be generated by known techniques. For example, Fab and F(ab′)₂ fragments of the disclosure may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CH1 domains of the heavy chains, and a disulfide-forming hinge region of the parent IgG molecule. Accordingly, an F(ab′)₂ fragment is still capable of crosslinking antigen molecules like the parent IgG molecule.

(2) Anti-CK-MB, β-hCG, TSH, Homocysteine, or free T4 Monoclonal Antibodies Using SLAM

In another aspect of the disclosure, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93:7843-7848 (1996). In this method, single cells secreting antibodies of interest, e.g., lymphocytes derived from any one of the immunized animals are screened using an antigen-specific hemolytic plaque assay, wherein the antigen CK-MB, β-hCG, TSH, homocysteine, or free T4, a subunit of CK-MB, β-hCG, TSH, homocysteine, or free T4, or a fragment thereof, is coupled to sheep red blood cells using a linker, such as biotin, and used to identify single cells that secrete antibodies with specificity for CK-MB, β-hCG, TSH, homocysteine, or free T4. Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to CK-MB, β-hCG, TSH, homocysteine, or free T4. The amplified immunoglobulin sequences further can be manipulated in vitro, such as by in vitro affinity maturation method. See, for example, PCT Publication No. WO 97/29131 and PCT Publication No. WO 00/56772.

(3) Anti-CK-MB, β-hCG, TSH, Homocysteine, or free T4 Monoclonal Antibodies Using Transgenic Animals

In another embodiment of the disclosure, antibodies are produced by immunizing a non-human animal comprising some, or all, of the human immunoglobulin locus with a CK-MB, β-hCG, TSH, homocysteine, or free T4 antigen. In an embodiment, the non-human animal is a XENOMOUSE® transgenic mouse, an engineered mouse strain that comprises large fragments of the human immunoglobulin loci and is deficient in mouse antibody production. See, e.g., Green et al., Nature Genetics, 7:13-21 (1994) and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies, and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al., Nature Genetics, 15:146-156 (1997), Green and Jakobovits, J. Exp. Med., 188:483-495 (1998), the disclosures of which are hereby incorporated by reference.

(4) Anti-CK-MB, β-hCG, TSH, Homocysteine, or free T4 Monoclonal Antibodies Using Recombinant Antibody Libraries

In vitro methods also can be used to make the antibodies of the disclosure, wherein an antibody library is screened to identify an antibody having the desired CK-MB, β-hCG, TSH, homocysteine, or free T4-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9:1369-1372 (1991); Hay et al., Hum. Antibod. Hybridomas, 3:81-85 (1992); Huse et al., Science, 246:1275-1281 (1989); McCafferty et al., Nature, 348:552-554 (1990); Griffiths et al., EMBO J., 12:725-734 (1993); Hawkins et al., J. Mol. Biol., 226:889-896 (1992); Clackson et al., Nature, 352:624-628 (1991); Gram et al., Proc. Natl. Acad. Sci. USA, 89:3576-3580 (1992); Garrard et al., Bio/Technology, 9:1373-1377 (1991); Hoogenboom et al., Nucl. Acids Res., 19:4133-4137 (1991); Barbas et al., Proc. Natl. Acad. Sci. USA, 88:7978-7982 (1991); U.S. Patent Application Publication No. 2003/0186374; and PCT Publication No. WO 97/29131, the contents of each of which are incorporated herein by reference.

The recombinant antibody library may be from a subject immunized with CK-MB, β-hCG, TSH, homocysteine, or free T4, or a portion of CK-MB, β-hCG, TSH, homocysteine, or free T4. Alternatively, the recombinant antibody library may be from a naive subject, i.e., one who has not been immunized with CK-MB, β-hCG, TSH, homocysteine, or free T4, such as a human antibody library from a human subject who has not been immunized with human CK-MB, β-hCG, TSH, homocysteine, or free T4. Antibodies of the disclosure are selected by screening the recombinant antibody library with the peptide comprising human CK-MB, β-hCG, TSH, homocysteine, or free T4 to thereby select those antibodies that recognize CK-MB, β-hCG, TSH, homocysteine, or free T4. Methods for conducting such screening and selection are well known in the art, such as described in the references in the preceding paragraph. To select antibodies of the disclosure having particular binding affinities for CK-MB, β-hCG, TSH, homocysteine, or free T4, such as those that dissociate from human CK-MB, β-hCG, TSH, homocysteine, or free T4 with a particular K_{off} rate constant, the art-known method of surface plasmon resonance can be used to select antibodies having the desired K_{off} rate constant. To select antibodies of the disclosure having a particular neutralizing activity for CK-MB, β-hCG, TSH, homocysteine, or free T4, such as those with a particular IC₅₀, standard methods known in the art for assessing the inhibition of CK-MB, β-hCG, TSH, homocysteine, or free T4 activity may be used.

In one aspect, the disclosure pertains to an isolated antibody, or an antigen-binding portion thereof, that binds human CK-MB, β-hCG, TSH, homocysteine, or free T4. Preferably, the antibody is a neutralizing antibody. In various embodiments, the antibody is a recombinant antibody or a monoclonal antibody.

For example, antibodies can also be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used to make the antibodies include those disclosed in Brinkmann et al., J. Immunol. Methods, 182:41-50 (1995); Ames et al., J. Immunol. Methods, 184:177-186 (1995); Kettleborough et al., Eur. J. Immunol., 24:952-958 (1994); Persic et al., Gene, 187:9-18 (1997); Burton et al., Advances in Immunology, 57:191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments can also be employed using methods known in the art such as those disclosed in PCT publication No. WO 92/22324; Mullinax et al., BioTechniques, 12 (6): 864-869 (1992); Sawai et al., Am. J. Reprod. Immunol., 34:26-34 (1995); and Better et al., Science, 240:1041-1043 (1988). Examples of techniques which can be used to produce single-chain Fvs and antibodies include those described in U.S. Pat. Nos. 4,946,778 and 5,258,498; Huston et al., Methods in Enzymology, 203:46-88 (1991); Shu et al., Proc. Natl. Acad. Sci. USA, 90:7995-7999 (1993); and Skerra et al., Science, 240:1038-1041 (1988).

Alternative to screening of recombinant antibody libraries by phage display, other methodologies known in the art for screening large combinatorial libraries can be applied to the identification of antibodies of the disclosure. One type of alternative expression system is one in which the recombinant antibody library is expressed as RNA-protein fusions, as described in PCT Publication No. WO 98/31700 (Szostak and Roberts), and in Roberts and Szostak, Proc. Natl. Acad. Sci. USA, 94:12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies can also be generated using yeast display methods known in the art. In yeast display methods, genetic methods are used to tether antibody domains to the yeast cell wall and display them on the surface of yeast. Such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

c. Production of Recombinant CK-MB, β-hCG, TSH, homocysteine, or free T4 Antibodies

Antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the disclosure in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies of the disclosure include Chinese Hamster Ovary (CHO cells) (including dhfr-CHO cells, described in Urlaub and Chasin, Proc. Natl. Acad. Sci. USA, 77:4216-4220 (1980), used with a DHFR selectable marker, e.g., as described in Kaufman and Sharp, J. Mol. Biol., 159:601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody of this disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the disclosure. In addition, bifunctional antibodies may be produced in which one heavy and one light chain are an antibody of the disclosure (i.e., binds human CK-MB, β-hCG, TSH, homocysteine, or free T4) and the other heavy and light chain are specific for an antigen other than human CK-MB, β-hCG, TSH, homocysteine, or free T4 by crosslinking an antibody of the disclosure to a second antibody by standard chemical crosslinking methods.

In a preferred system for recombinant expression of an antibody, or antigen-binding portion thereof, of the disclosure, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the disclosure provides a method of synthesizing a recombinant antibody of the disclosure by culturing a host cell of the disclosure in a suitable culture medium until a recombinant antibody of the disclosure is synthesized. The method can further comprise isolating the recombinant antibody from the culture medium.

### (1) Humanized Antibody

The humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one aspect, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG 1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present disclosure can be performed using any known method, such as but not limited to those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for CK-MB, β-hCG, TSH, homocysteine, or free T4 and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristics, such as increased affinity for CK-MB, β-hCG, TSH, homocysteine, or free T4, is achieved. In general, the hypervariable region residues may be directly and most substantially involved in influencing antigen binding.

As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g. mice that are capable, upon immunization, of producing a full repertoire of human antibodies in the absence of endogenous immunoglobulin production. For example, the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

d. Anti-CK-MB, β-hCG, TSH, Homocysteine, or Free T4 antibodies

Anti-CK-MB, β-hCG, TSH, homocysteine, or free T4 antibodies may be generated using the techniques described above as well as using routine techniques known in the art. In some embodiments, the anti-CK-MB, β-hCG, TSH, homocysteine, or free T4 antibody may be an unconjugated CK-MB, β-hCG, TSH, homocysteine, or free T4 antibody, available from commercially available sources, such as, for example, those listed in the below Table 2.

TABLE 2

| Biomarker | Vendor and Catalog Numbers |
|---|---|
| CK-MB | 1. ThermoFisher Scientific - Creatine Kinase MB Rabbit Monoclonal |
| | Antibody (JB78-34)- Catalog No. MA5-34689; Creatine Kinase MB |
| | Monoclonal Antibody - Catalog No. MA5-30292. |
| | 2. Abcam - Anti-Creatine Kinase MB antibody [CK1] - Catalog No. |
| | ab404; Anti-Creatine Kinase MB antibody [BD1937] - Catalog No. |
| | ab 19603. |
| | 3. ARCHITECT ® or iSTAT ® CK-MB Assay |
| β-hCG | 1. ThermoFisher Scientific - hCG beta Monoclonal Antibody (5H4-E2) - |
| | Catalog No. MA1-35020; hCG beta Monoclonal Antibody (P1G7F12) - |
| | Catalog No. MIH9827 and MA5-14703; HCG-β Recombinant Rabbit |
| | Monoclonal Antibody (HCGa, 2728R) - Catalog No. 1081-RBM4-P1 and |
| | 1081-RBM4-P1ABX. |
| | 2. Abcam - Anti-hCG Beta Antibody [5H4-E2] - Catalog No. ab9852. |
| | 3. Abbott iSTAT ® Beta hCG Assay |
| TSH | 1. ThermoFisher Scientific - TSH Monoclonal Antibody (M1A10) - |
| | Catalog No. 600-570; TSH Monoclonal Antibody (1H4G9) - Catalog No. |
| | A01709-1; TSH Monoclonal Antibody (2B9H4) - Catalog No. A01711-1. |
| | 2. Abcam - Anti-TSH Beta Antibody [11E4] - Catalog No. ab6069; Anti- |
| | TSH Beta Antibody [EPR8197) - Catalog No. ab155959. |
| | 3. ARCHITECT ® TSH Assay |
| Homocysteine | 1. Abcam - Recombinant Anti-Adenosyl Homocysteine Antibody |
| | [EPR4499] - Catalog No. ab111903. |
| | 2. MyBiosource - Rabbit Homocysteine Polyclonal Antibody - Catalog |
| | No. MBS621964. |
| | 3.Alinity i Homocysteine Assay |
| Free T4 | 1. MyBiosource - Thyroxine (T4) Monoclonal Antibody - Catalog No. |
| | MBS310717. |
| | 2. Pantex - Monoclonal Anti-Thyroxine (T4) - Clone 21. |
| | 3. ThermoFisher Scientific - L-Thyroxine T4 Monoclonal Antibody |
| | (T4YCH) - Catalog No. MA5-14716. |
| | 4. ARCHITECT ® Free T4 Assay |

### 11. Variations on Methods

The disclosed methods of determining the presence or amount of analyte of interest (UCH-L1, GFAP, CK-MB, β-hCG, TSH, homocysteine, free T4 or any combinations thereof) present in a sample may be as described herein. The methods may also be adapted in view of other methods for analyzing analytes. Examples of well-known variations include, but are not limited to, immunoassay, such as sandwich immunoassay (e.g., monoclonal-monoclonal sandwich immunoassays, monoclonal-polyclonal sandwich immunoassays, including enzyme detection (enzyme immunoassay (EIA) or enzyme-linked immunosorbent assay (ELISA), competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay, heterogeneous assay, capture on the fly assay, etc.

a. Immunoassay

The analyte of interest, and/or peptides of fragments thereof (e.g., UCH-L1, GFAP, CK-MB, β-hCG, TSH, homocysteine, and/or free T4, and/or peptides or fragments thereof, i.e., UCH-L1, GFAP, CK-MB, β-hCG, TSH, homocysteine, and/or free T4, fragments), may be analyzed using UCH-L1, GFAP, CK-MB, β-hCG, TSH, homocysteine, and/or free T4, antibodies in an immunoassay. The presence or amount of analyte (e.g., UCH-L1, GFAP, CK-MB, β-hCG, TSH, homocysteine, and/or free T4) can be determined using antibodies and detecting specific binding to the analyte (e.g., UCH-L1, GFAP, CK-MB, β-hCG, TSH, homocysteine, and/or free T4). For example, the antibody, or antibody fragment thereof, may specifically bind to the analyte (e.g., UCH-L1, GFAP, CK-MB, β-hCG, TSH, homocysteine, and/or free T4). If desired, one or more of the antibodies can be used in combination with one or more commercially available monoclonal/polyclonal antibodies. Such antibodies are available from companies such as R&D Systems, Inc. (Minneapolis, MN) and Enzo Life Sciences International, Inc. (Plymouth Meeting, PA).

The presence or amount of analyte (e.g., UCH-L1, GFAP, CK-MB, β-hCG, TSH, homocysteine, and/or free T4) present in a body sample may be readily determined using an immunoassay, such as sandwich immunoassay (e.g., monoclonal-monoclonal sandwich immunoassays, monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, MN)). An example of a point-of-care device that can be used is i-STAT® (Abbott, Laboratories, Abbott Park, IL). Other methods that can be used include a chemiluminescent microparticle immunoassay, in particular one employing the ARCHITECT® automated analyzer (Abbott Laboratories, Abbott Park, IL), as an example. Other methods include, for example, mass spectrometry, and immunohistochemistry (e.g., with sections from tissue biopsies), using anti-analyte (e.g., UCH-L1, GFAP, CK-MB, β-hCG, TSH, homocysteine, and/or free T4) antibodies (monoclonal, polyclonal, chimeric, humanized, human, etc.) or antibody fragments thereof against analyte (e.g., UCH-L1, GFAP, CK-MB, β-hCG, TSH, homocysteine, and/or free T4). Other methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Specific immunological binding of the antibody to the analyte (e.g., UCH-L1, GFAP, CK-MB, β-hCG, TSH, homocysteine, and/or free T4) can be detected via direct labels, such as fluorescent or luminescent tags, metals and radionuclides attached to the antibody or via indirect labels, such as alkaline phosphatase or horseradish peroxidase.

The use of immobilized antibodies or antibody fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test sample and processed quickly through washes and detection steps to generate a measurable signal, such as a colored spot.

A homogeneous format may be used. For example, after the test sample is obtained from a subject, a mixture is prepared. The mixture contains the test sample being assessed for analyte (e.g., UCH-L1, GFAP, CK-MB, β-hCG, TSH, homocysteine, and/or free T4), a first specific binding partner, and a second specific binding partner. The order in which the test sample, the first specific binding partner, and the second specific binding partner are added to form the mixture is not critical. The test sample is simultaneously contacted with the first specific binding partner and the second specific binding partner. In some embodiments, the first specific binding partner and any UCH-L1, GFAP, CK-MB, β-hCG, TSH, homocysteine, and/or free T4, contained in the test sample may form a first specific binding partner-analyte (e.g., UCH-L1, GFAP, CK-MB, β-hCG, TSH, homocysteine, and/or free T4)-antigen complex and the second specific binding partner may form a first specific binding partner-analyte of interest (e.g., UCH-L1, GFAP, CK-MB, β-hCG, TSH, homocysteine, and/or free T4)-second specific binding partner complex. In some embodiments with respect to UCH-L1 and/or GFAP, the second specific binding partner and any UCH-L1 and/or GFAP contained in the test sample may form a second specific binding partner-analyte (e.g., UCH-L1)-antigen complex and the first specific binding partner may form a first specific binding partner-analyte of interest (e.g., UCH-L1 and/or GFAP)-second specific binding partner complex. The first specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). The second specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). Moreover, the second specific binding partner is labeled with or contains a detectable label as described above.

A heterogeneous format may be used. For example, after the test sample is obtained from a subject, a first mixture is prepared. The mixture contains the test sample being assessed for analyte (e.g., UCH-L1, GFAP, CK-MB, β-hCG, TSH, homocysteine, and/or free T4) and a first specific binding partner, wherein the first specific binding partner and any UCH-L1, GFAP, CK-MB, β-hCG, TSH, homocysteine, and/or free T4, contained in the test sample form a first specific binding partner-analyte (e.g., UCH-L1, GFAP, CK-MB, β-hCG, TSH, homocysteine, and/or free T4)-antigen complex. In some aspects with respect to UCH-L1 and/or GFAP, the first specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). The order in which the test sample and the first specific binding partner are added to form the mixture is not critical.

The first specific binding partner may be immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc, and a chip. In those embodiments where the solid phase is a bead, the bead may be a magnetic bead or a magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO·Fe₂O₃). Beads can have a solid core portion that is magnetic and is surrounded by one or more non-magnetic layers. Alternately, the magnetic portion can be a layer around a non-magnetic core. The solid support on which the first specific binding member is immobilized may be stored in dry form or in a liquid. The magnetic beads may be subjected to a magnetic field prior to or after contacting with the sample with a magnetic bead on which the first specific binding member is immobilized.

After the mixture containing the first specific binding partner-analyte (e.g., UCH-L1, GFAP, CK-MB, β-hCG, TSH, homocysteine, and/or free T4) antigen complex is formed, any unbound analyte (e.g., UCH-L1, GFAP, CK-MB, β-hCG, TSH, homocysteine, and/or free T4) is removed from the complex using any technique known in the art. For example, the unbound analyte (e.g., UCH-L1, GFAP, CK-MB, β-hCG, TSH, homocysteine, and/or free T4) can be removed by washing. Desirably, however, the first specific binding partner is present in excess of any analyte (e.g., UCH-L1, GFAP, CK-MB, β-hCG, TSH, homocysteine, and/or free T4) present in the test sample, such that all analyte (e.g., UCH-L1, GFAP, CK-MB, β-hCG, TSH, homocysteine, and/or free T4) that is present in the test sample is bound by the first specific binding partner.

After any unbound analyte (e.g., UCH-L1, GFAP, CK-MB, β-hCG, TSH, homocysteine, and/or free T4) is removed, a second specific binding partner is added to the mixture to form a first specific binding partner-analyte of interest (e.g., UCH-L1, GFAP, CK-MB, β-hCG, TSH, homocysteine, and/or free T4)-second specific binding partner complex. In some aspects with respect to UCH-L1 and GFAP, the second specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). Moreover, the second specific binding partner is labeled with or contains a detectable label as described above.

The use of immobilized antibodies or antibody fragments thereof may be incorporated into the immunoassay. The antibodies may be immobilized onto a variety of supports, such as magnetic or chromatographic matrix particles (such as a magnetic bead), latex particles or modified surface latex particles, polymer or polymer film, plastic or plastic film, planar substrate, the surface of an assay plate (such as microtiter wells), pieces of a solid substrate material, and the like. An assay strip can be prepared by coating the antibody or plurality of antibodies in an array on a solid support. This strip can then be dipped into the test sample and processed quickly through washes and detection steps to generate a measurable signal, such as a colored spot.

In some aspects, it is possible that other antibodies can be selected which similarly may assist with maintaining the dynamic range and low end sensitivity of the immunoassays. For example, it may be useful to select at least one first antibody (such as a capture antibody or first specific binding partner) that binds to an epitope near the N-terminus of the 38 kDa BDP and at least one second antibody (such as a detection antibody or second specific binding partner) that binds to an epitope near the middle of the 38 kDa BDP, e.g., near the middle of the 38 kDa BDP, and that does not overlap with the first antibody. Other variations are possible and could be readily tested by one of ordinary skill, such as by confirming antibodies bind to different epitopes by examining binding to short peptides, and then screening antibody pairs using low calibrator concentration. Moreover, selecting antibodies of differing affinity for GFAP also can assist with maintaining or increasing the dynamic range of the assay. GFAP antibodies have been described in the literature and are commercially available.

(1) Sandwich immunoassay

A sandwich immunoassay measures the amount of antigen between two layers of antibodies (i.e., at least one capture antibody) and a detection antibody (i.e., at least one detection antibody). The capture antibody and the detection antibody bind to different epitopes on the antigen, e.g., analyte of interest such as UCH-L1, GFAP, CK-MB, β-hCG, TSH, homocysteine, and/or free T4. Desirably, binding of the capture antibody to an epitope does not interfere with binding of the detection antibody to an epitope. Either monoclonal or polyclonal antibodies may be used as the capture and detection antibodies in the sandwich immunoassay.

Generally, at least two antibodies are employed to separate and quantify analyte (e.g., UCH-L1, GFAP, CK-MB, β-hCG, TSH, homocysteine, and/or free T4) in a test sample. More specifically, the at least two antibodies bind to certain epitopes of analyte (e.g., UCH-L1, GFAP, CK-MB, β-hCG, TSH, homocysteine, and/or free T4) forming an immune complex which is referred to as a “sandwich”. One or more antibodies can be used to capture the analyte (e.g., UCH-L1, GFAP, CK-MB, β-hCG, TSH, homocysteine, and/or free T4) in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, the binding of an antibody to its epitope desirably is not diminished by the binding of any other antibody in the assay to its respective epitope. Antibodies are selected so that the one or more first antibodies brought into contact with a test sample suspected of containing analyte (e.g., UCH-L1, GFAP, CK-MB, β-hCG, TSH, homocysteine, and/or free T4) do not bind to all or part of an epitope recognized by the second or subsequent antibodies, thereby interfering with the ability of the one or more second detection antibodies to bind to the analyte (e.g., UCH-L1, GFAP, CK-MB, β-hCG, TSH, homocysteine, and/or free T4).

The antibodies may be used as a first antibody in said immunoassay. The antibody immunospecifically binds to epitopes on analyte (e.g., UCH-L1, GFAP, CK-MB, β-hCG, TSH, homocysteine, and/or free T4). In addition to the antibodies of the present disclosure, said immunoassay may comprise a second antibody that immunospecifically binds to epitopes that are not recognized or bound by the first antibody.

A test sample suspected of containing analyte (e.g., UCH-L1, GFAP, CK-MB, β-hCG, TSH, homocysteine, and/or free T4) can be contacted with at least one first capture antibody (or antibodies) and at least one second detection antibodies either simultaneously or sequentially. In the sandwich assay format, a test sample suspected of containing analyte (e.g., UCH-L1, GFAP, CK-MB, β-hCG, TSH, homocysteine, and/or free T4) is first brought into contact with the at least one first capture antibody that specifically binds to a particular epitope under conditions which allow the formation of a first antibody-analyte (e.g., UCH-L1, GFAP, CK-MB, β-hCG, TSH, homocysteine, and/or free T4) antigen complex. If more than one capture antibody is used, a first multiple capture antibody-UCH-L1, GFAP, CK-MB, β-hCG, TSH, homocysteine, and/or free T4 antigen complex is formed. In a sandwich assay, the antibodies, preferably, the at least one capture antibody, are used in molar excess amounts of the maximum amount of analyte (e.g., UCH-L1, GFAP, CK-MB, β-hCG, TSH, homocysteine, and/or free T4) expected in the test sample. For example, from about 5 μg/mL to about 1 mg/mL of antibody per ml of microparticle coating buffer may be used.

i. Capture Antibodies

Optionally, prior to contacting the test sample with the at least one first capture antibody, the at least one first capture antibody can be bound to a solid support which facilitates the separation the first antibody-analyte (e.g., UCH-L1, GFAP, CK-MB, β-hCG, TSH, homocysteine, and/or free T4) complex from the test sample. Any solid support known in the art can be used, including but not limited to, solid supports made out of polymeric materials in the forms of wells, tubes, or beads (such as a microparticle). The antibody (or antibodies) can be bound to the solid support by adsorption, by covalent bonding using a chemical coupling agent or by other means known in the art, provided that such binding does not interfere with the ability of the antibody to bind analyte (e.g., UCH-L1, GFAP, CK-MB, β-hCG, TSH, homocysteine, and/or free T4). Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl) carbodiimide.

After the test sample suspected of containing analyte (e.g., UCH-L1, GFAP, CK-MB, β-hCG, TSH, homocysteine, and/or free T4) is incubated in order to allow for the formation of a first capture antibody (or multiple antibody)-analyte (e.g., UCH-L1, GFAP, CK-MB, β-hCG, TSH, homocysteine, and/or free T4) complex. The incubation can be carried out at a pH of from about 4.5 to about 10.0, at a temperature of from about 2° C. to about 45° C., and for a period from at least about one (1) minute to about eighteen (18) hours, from about 2-6 minutes, from about 7-12 minutes, from about 5-15 minutes, or from about 3-4 minutes.

ii. Detection Antibodies

After formation of the first/multiple capture antibody-analyte (e.g., UCH-L1, GFAP, CK-MB, β-hCG, TSH, homocysteine, and/or free T4) complex, the complex is then contacted with at least one second detection antibody (under conditions that allow for the formation of a first/multiple antibody-analyte (e.g., UCH-L1, GFAP, CK-MB, -hCG, TSH, homocysteine, and/or free T4) antigen-second antibody complex). In some embodiments, the test sample is contacted with the detection antibody simultaneously with the capture antibody. If the first antibody-analyte (e.g., UCH-L1, GFAP, CK-MB, β-hCG, TSH, homocysteine, and/or free T4) complex is contacted with more than one detection antibody, then a first/multiple capture antibody-analyte (e.g., UCH-L1, GFAP, CK-MB, β-hCG, TSH, homocysteine, and/or free T4)-multiple antibody detection complex is formed. As with first antibody, when the at least second (and subsequent) antibody is brought into contact with the first antibody-analyte (e.g., UCH-L1, GFAP, CK-MB, β-hCG, TSH, homocysteine, and/or free T4) complex, a period of incubation under conditions similar to those described above is required for the formation of the first/multiple antibody-analyte (e.g., UCH-L1, GFAP, CK-MB, β-hCG, TSH, homocysteine, and/or free T4)-second/multiple antibody complex. Preferably, at least one second antibody contains a detectable label. The detectable label can be bound to the at least one second antibody prior to, simultaneously with or after the formation of the first/multiple antibody-analyte (e.g., UCH-L1, GFAP, CK-MB, β-hCG, TSH, homocysteine, and/or free T4)-second/multiple antibody complex. Any detectable label known in the art can be used.

Chemiluminescent assays can be performed in accordance with the methods described in Adamczyk et al., Anal. Chim. Acta 579 (1): 61-67 (2006). While any suitable assay format can be used, a microplate chemiluminometer (Mithras LB-940, Berthold Technologies U.S.A., LLC, Oak Ridge, TN) enables the assay of multiple samples of small volumes rapidly. The chemiluminometer can be equipped with multiple reagent injectors using 96-well black polystyrene microplates (Costar #3792). Each sample can be added into a separate well, followed by the simultaneous/sequential addition of other reagents as determined by the type of assay employed. Desirably, the formation of pseudobases in neutral or basic solutions employing an acridinium aryl ester is avoided, such as by acidification. The chemiluminescent response is then recorded well-by-well. In this regard, the time for recording the chemiluminescent response will depend, in part, on the delay between the addition of the reagents and the particular acridinium employed.

The order in which the test sample and the specific binding partner(s) are added to form the mixture for chemiluminescent assay is not critical. If the first specific binding partner is detectably labeled with an acridinium compound, detectably labeled first specific binding partner-antigen (e.g., UCH-L1, GFAP, CK-MB, β-hCG, TSH, homocysteine, and/or free T4) complexes form. Alternatively, if a second specific binding partner is used and the second specific binding partner is detectably labeled with an acridinium compound, detectably labeled first specific binding partner-analyte (e.g., UCH-L1, GFAP, CK-MB, β-hCG, TSH, homocysteine, and/or free T4)-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture before, simultaneously with, or after the addition of an above-described acridinium compound. Hydrogen peroxide can be generated in situ in a number of ways such as would be apparent to one skilled in the art.

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the simultaneous or subsequent addition of at least one basic solution to the sample, a detectable signal, namely, a chemiluminescent signal, indicative of the presence of analyte (e.g., UCH-L1, GFAP, CK-MB, β-hCG, TSH, homocysteine, and/or free T4) is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample. Other labels other than chemiluminescent labels can be employed. For instance, enzymatic labels (including but not limited to alkaline phosphatase) can be employed.

The chemiluminescent signal, or other signal, that is generated can be detected using routine techniques known to those skilled in the art. Based on the intensity of the signal generated, the amount of analyte of interest (e.g., UCH-L1, GFAP, CK-MB, β-hCG, TSH, homocysteine, and/or free T4) in the sample can be quantified. Specifically, the amount of analyte (e.g., UCH-L1, GFAP, CK-MB, β-hCG, TSH, homocysteine, and/or free T4) in the sample is proportional to the intensity of the signal generated. The amount of analyte (e.g., UCH-L1, GFAP, CK-MB, β-hCG, TSH, homocysteine, and/or free T4) present can be quantified by comparing the amount of light generated to a standard curve for analyte (e.g., UCH-L1, GFAP, CK-MB, β-hCG, TSH, homocysteine, and/or free T4) or by comparison to a reference standard. The standard curve can be generated using serial dilutions or solutions of known concentrations of analyte (e.g., UCH-L1, GFAP, CK-MB, β-hCG, TSH, homocysteine, and/or free T4) by mass spectroscopy, gravimetric methods, and other techniques known in the art.

### (2) Forward Competitive Inhibition Assay

In a forward competitive format, an aliquot of labeled analyte of interest (e.g., analyte (e.g., UCH-L1, GFAP, CK-MB, β-hCG, TSH, homocysteine, and/or free T4 having a fluorescent label, a tag attached with a cleavable linker, etc.) of a known concentration is used to compete with analyte of interest (e.g., UCH-L1, GFAP, CK-MB, β-hCG, TSH, homocysteine, and/or free T4) in a test sample for binding to analyte of interest antibody (e.g., UCH-L1, GFAP, CK-MB, β-hCG, TSH, homocysteine, and/or free T4).

In a forward competition assay, an immobilized specific binding partner (such as an antibody) can either be sequentially or simultaneously contacted with the test sample and a labeled analyte of interest, analyte of interest fragment or analyte of interest variant thereof. The analyte of interest peptide, analyte of interest fragment or analyte of interest variant can be labeled with any detectable label, including a detectable label comprised of tag attached with a cleavable linker. In this assay, the antibody can be immobilized on to a solid support. Alternatively, the antibody can be coupled to an antibody, such as an anti-species antibody, that has been immobilized on a solid support, such as a microparticle or planar substrate.

The labeled analyte of interest, the test sample and the antibody are incubated under conditions similar to those described above in connection with the sandwich assay format. Two different species of antibody-analyte of interest complexes may then be generated. Specifically, one of the antibody-analyte of interest complexes generated contains a detectable label (e.g., a fluorescent label, etc.) while the other antibody-analyte of interest complex does not contain a detectable label. The antibody-analyte of interest complex can be, but does not have to be, separated from the remainder of the test sample prior to quantification of the detectable label. Regardless of whether the antibody-analyte of interest complex is separated from the remainder of the test sample, the amount of detectable label in the antibody-analyte of interest complex is then quantified. The concentration of analyte of interest (such as membrane-associated analyte of interest, soluble analyte of interest, fragments of soluble analyte of interest, variants of analyte of interest (membrane-associated or soluble analyte of interest) or any combinations thereof) in the test sample can then be determined, e.g., as described above.

### (3) Reverse Competitive Inhibition Assay

In a reverse competition assay, an immobilized analyte of interest (e.g., UCH-L1, GFAP, CK-MB, β-hCG, TSH, homocysteine, and/or free T4) can either be sequentially or simultaneously contacted with a test sample and at least one labeled antibody.

The analyte of interest can be bound to a solid support, such as the solid supports discussed above in connection with the sandwich assay format.

The immobilized analyte of interest, test sample and at least one labeled antibody are incubated under conditions similar to those described above in connection with the sandwich assay format. Two different species analyte of interest-antibody complexes are then generated. Specifically, one of the analyte of interest-antibody complexes generated is immobilized and contains a detectable label (e.g., a fluorescent label, etc.) while the other analyte of interest-antibody complex is not immobilized and contains a detectable label. The non-immobilized analyte of interest-antibody complex and the remainder of the test sample are removed from the presence of the immobilized analyte of interest-antibody complex through techniques known in the art, such as washing. Once the non-immobilized analyte of interest antibody complex is removed, the amount of detectable label in the immobilized analyte of interest-antibody complex is then quantified following cleavage of the tag. The amount or concentration of analyte of interest in the test sample can then be determined by comparing the quantity of detectable label as described above.

### (4) One-Step Immunoassay or “Capture on the Fly” Assay

In a capture on the fly immunoassay, a solid substrate is pre-coated with an immobilization agent. The capture agent, the analyte (e.g., UCH-L1, GFAP, CK-MB, β-hCG, TSH, homocysteine, and/or free T4) and the detection agent are added to the solid substrate together, followed by a wash step prior to detection. The capture agent can bind the analyte (e.g., UCH-L1, GFAP, CK-MB, β-hCG, TSH, homocysteine, and/or free T4) and comprises a ligand for an immobilization agent. The capture agent and the detection agents may be antibodies or any other moiety capable of capture or detection as described herein or known in the art. The ligand may comprise a peptide tag and an immobilization agent may comprise an anti-peptide tag antibody. Alternately, the ligand and the immobilization agent may be any pair of agents capable of binding together so as to be employed for a capture on the fly assay (e.g., specific binding pair, and others such as are known in the art). More than one analyte may be measured. In some embodiments, the solid substrate may be coated with an antigen and the analyte to be analyzed is an antibody.

In certain other embodiments, in a one-step immunoassay or “capture on the fly”, a solid support (such as a microparticle) pre-coated with an immobilization agent (such as biotin, streptavidin, etc.) and at least a first specific binding member and a second specific binding member (which function as capture and detection reagents, respectively) are used. The first specific binding member comprises a ligand for the immobilization agent (for example, if the immobilization agent on the solid support is streptavidin, the ligand on the first specific binding member may be biotin) and also binds to the analyte of interest (e.g., UCH-L1, GFAP, CK-MB, β-hCG, TSH, homocysteine, and/or free T4). The second specific binding member comprises a detectable label and binds to an analyte of interest (e.g., UCH-L1, GFAP, CK-MB, β-hCG, TSH, homocysteine, and/or free T4). The solid support and the first and second specific binding members may be added to a test sample (either sequentially or simultaneously). The ligand on the first specific binding member binds to the immobilization agent on the solid support to form a solid support/first specific binding member complex. Any analyte of interest present in the sample binds to the solid support/first specific binding member complex to form a solid support/first specific binding member/analyte complex. The second specific binding member binds to the solid support/first specific binding member/analyte complex and the detectable label is detected. An optional wash step may be employed before the detection. In certain embodiments, in a one-step assay more than one analyte may be measured. In certain other embodiments, more than two specific binding members can be employed. In certain other embodiments, multiple detectable labels can be added. In certain other embodiments, multiple analytes of interest can be detected, or their amounts, levels or concentrations, measured, determined or assessed.

The use of a capture on the fly assay can be done in a variety of formats as described herein, and known in the art. For example, the format can be a sandwich assay such as described above, but alternately can be a competition assay, can employ a single specific binding member, or use other variations such as are known.

### 4. Other Factors

The methods of diagnosing, prognosticating, and/or assessing, as described above, can further include using other factors for the diagnosis, prognostication, and assessment. In some embodiments, traumatic brain injury may be diagnosed using the Glasgow Coma Scale. Other tests, scales or indices can also be used either alone or in combination with the Glasgow Coma Scale. An example is the Ranchos Los Amigos Scale. The Ranchos Los Amigos Scale measures the levels of awareness, cognition, behavior and interaction with the environment. The Ranchos Los Amigos Scale includes: Level I: No Response; Level II: Generalized Response; Level III: Localized Response; Level IV: Confused-agitated; Level V: Confused-inappropriate; Level VI: Confused-appropriate; Level VII: Automatic-appropriate; and Level VIII: Purposeful-appropriate. Another example is the Rivermead Post-Concussion Symptoms Questionairre, a self-report scale to measure the severity of post-concussive symptoms following TBL. Patients are asked to rate how severe each of 16 symptoms (e.g., headache, dizziness, nausea, vomiting) has been over the past 24 hours. In each case, the symptom is compared with how severe it was before the injury occurred (premorbid). These symptoms are reported by severity on a scale from 0 to 4: not experienced, no more of a problem, mild problem, moderate problem, and severe problem.

### 12. Samples

### TBI Biomarkers

In some embodiments, the sample is obtained after the subject, such as a human subject, sustained an injury to the head caused by physical shaking, blunt impact by an external mechanical or other force that results in a closed or open head trauma, one or more falls, explosions or blasts or other types of blunt force trauma. In some embodiments, the sample is obtained after the subject, such as a human subject, has ingested or been exposed to a fire, chemical, toxin or combination of a fire, chemical and toxin. Examples of such chemicals and/or toxins include, molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. In some embodiments, the sample is obtained from a subject, such as a human subject, that suffers from an autoimmune disease, a metabolic disorder, a brain tumor, hypoxia, a viral infection (e.g., SARS-COV-2), a fungal infection, a bacterial infection, meningitis, hydrocephalus, or any combinations thereof.

In yet another embodiment, the methods described herein use samples that also can be used to determine whether or not a subject has or is at risk of developing a TBI (such as a mild TBI, moderate TBI, severe TBI, or moderate to severe TBI) by determining the levels of UCH-L1 and/or GFAP in a subject using the anti-UCH-L1 and/or anti-GFAP antibodies described below, or antibody fragments thereof. Thus, in particular embodiments, the disclosure also provides a method for determining whether a subject having, or at risk for, traumatic brain injuries, discussed herein and known in the art, is a candidate for therapy or treatment. Generally, the subject is at least one who: (i) has experienced an injury to the head; (ii) ingested and/or been exposed to one or more chemicals and/or toxins; (iii) suffers from an autoimmune disease, a metabolic disorder, a brain tumor, hypoxia, a viral infection (e.g., SARS-COV-2), a fungal infection, a bacterial infection, meningitis, hydrocephalus, or any combinations thereof; or (iv) any combinations of (i)-(iii); or, who has actually been diagnosed as having, or being at risk for TBI (such as, for example, subjects suffering from an autoimmune disease, a metabolic disorder, a brain tumor, hypoxia, a viral infection (e.g., SARS-COV-2), a fungal infection, a bacterial infection, meningitis, hydrocephalus, or any combinations thereof), and/or who demonstrates an unfavorable (i.e., clinically undesirable) concentration or amount of UCH-L1 and/or GFAP or UCH-L1 and/or GFAP fragment, as described herein.

### Other Biomarkers

In yet another embodiment, the methods described herein use samples that also can be used to determine whether or not a subject has or is at risk of developing a myocardial infarction by determining the level of CK-MB using the anti-CK-MB antibodies described herein, or antibody fragments thereof. In still yet another embodiment, the methods described herein can also be used to determine whether or not a subject is pregnant by determining the level of β-hCG using the anti-β-hCG antibodies described herein, or antibody fragments thereof. In still yet another embodiment, the methods described herein use samples that also can be used to assess thyroid function in a subject, diagnosis thyroid disease in a subject and/or treat thyroid disease in a subject by determining the level of TSH using the anti-TSH antibodies described herein, or antibody fragments thereof. In still yet another embodiment, the methods described herein use samples that also can be used to diagnose hyperhomocysteinemia and/or homocystinuria in a subject or treat subjects having hyperhomocysteinemia and/or homocystinuria in a subject by determining the level of homocysteine using the anti-homocysteine antibodies described herein, or antibody fragments thereof. In still yet another embodiment, the methods described herein use samples that also can be used to assess thyroid function in a subject, diagnosis thyroid disease in a subject and/or treat thyroid disease in a subject by determining the level of free T4 using the anti-free T4 antibodies described herein, or antibody fragments thereof.

In still other aspects, the amount of TSH determined in the capillary blood sample obtained according to the methods described herein can be used to assess thyroid function in a subject, diagnosis thyroid disease in a subject and/or treat thyroid disease in a subject. In still other aspects, the amount of homocysteine determined in the capillary blood sample obtained according to the methods described herein can be used to diagnose hyperhomocysteinemia and/or homocystinuria in a subject or treat subjects having hyperhomocysteinemia and/or homocystinuria. In still other aspects, the amount of free T4 determined in the capillary blood sample obtained according to the methods described herein can be used to assess thyroid function in a subject, diagnosis thyroid disease in a subject, treat thyroid disease in a subject, or any combination thereof.

a. Controls

It may be desirable to include a control sample. The control sample may be analyzed concurrently with the sample from the subject as described above. The results obtained from the subject sample can be compared to the results obtained from the control sample. Standard curves may be provided, with which assay results for the sample may be compared. Such standard curves present levels of marker as a function of assay units, i.e., fluorescent signal intensity, if a fluorescent label is used. Using samples taken from multiple donors, standard curves can be provided for reference levels of the UCH-L1, GFAP, CK-MB, β-hCG, TSH, homocysteine, and/or free T4 in normal healthy subjects (e.g., in the case of β-hCG, subjects that are not pregnant) or tissue, as well as for “at-risk” levels of the UCH-L1, GFAP, CK-MB, β-hCG, TSH, homocysteine, and/or free T4 in tissue taken from donors, who may have one or more of the characteristics set forth above.

Thus, in view of the above, a method for determining the presence, amount, or concentration of UCH-L1, GFAP, CK-MB, β-hCG, TSH, homocysteine, and/or free T4 in a test sample is provided. The method comprises assaying the test sample for UCH-L1, GFAP, CK-MB, β-hCG, TSH, homocysteine, and/or free T4 by an immunoassay, for example, employing at least one capture antibody that binds to an epitope on UCH-L1, GFAP, CK-MB, β-hCG, TSH, homocysteine, and/or free T4 and at least one detection antibody that binds to an epitope on UCH-L1, GFAP, CK-MB, β-hCG, TSH, homocysteine, and/or free T4 which is different from the epitope for the capture antibody and optionally includes a detectable label, and comprising comparing a signal generated by the detectable label as a direct or indirect indication of the presence, amount or concentration of UCH-L1 and/or GFAP in the test sample to a signal generated as a direct or indirect indication of the presence, amount or concentration of UCH-L1, GFAP, CK-MB, β-hCG, TSH, homocysteine, and/or free T4 in a calibrator. The calibrator is optionally, and is preferably, part of a series of calibrators in which each of the calibrators differs from the other calibrators in the series by the concentration of UCH-L1, GFAP, CK-MB, β-hCG, TSH, homocysteine, and/or free T4.

### 13. Kit

Provided herein is a kit, which may be used for assaying or assessing a test sample for UCH-L1, GFAP, CK-MB, β-hCG, TSH, homocysteine, and/or free T4 fragment. The kit comprises at least one component for assaying the test sample for UCH-L1, GFAP, CK-MB, β-hCG, TSH, homocysteine, and/or free T4 instructions for assaying the test sample for UCH-L1, GFAP, CK-MB, β-hCG, TSH, homocysteine, and/or free T4. For example, the kit can comprise instructions for assaying the test sample for UCH-L1, GFAP, CK-MB, β-hCG, TSH, homocysteine, and/or free T4 by immunoassay, e.g., chemiluminescent microparticle immunoassay. Instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The at least one component may include at least one composition comprising one or more isolated antibodies or antibody fragments thereof that specifically bind to UCH-L1, GFAP, CK-MB, β-hCG, TSH, homocysteine, and/or free T4. The antibody may be a UCH-L1, GFAP, CK-MB, β-hCG, TSH, homocysteine, and/or free T4 capture antibody and/or a UCH-L1 and/or GFAP detection antibody.

Alternatively or additionally, the kit can comprise a calibrator or control, e.g., purified, and optionally lyophilized, UCH-L1, GFAP, CK-MB, β-hCG, TSH, homocysteine, and/or free T4, and/or at least one container (e.g., tube, microtiter plates or strips, which can be already coated with an anti-UCH-L1, GFAP, CK-MB, β-hCG, TSH, homocysteine, and/or free T4 monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve.

Moreover, the kit can further comprise a microsampling device, a plasma separation device and/or a microsampling device and a plasma separation device.

The kit may further comprise reference standards for quantifying UCH-L1, GFAP, CK-MB, β-hCG, TSH, homocysteine, and/or free T4. The reference standards may be employed to establish standard curves for interpolation and/or extrapolation of UCH-L1, GFAP, CK-MB, β-hCG, TSH, homocysteine, and/or free T4 concentrations. The reference standards may include a high UCH-L1, GFAP, CK-MB, β-hCG, TSH, homocysteine, and/or free T4 concentration level, for example, about 100000 μg/mL, about 125000 μg/mL, about 150000 μg/mL, about 175000 μg/mL, about 200000 μg/mL, about 225000 μg/mL, about 250000 μg/mL, about 275000 μg/mL, or about 300000 μg/mL; a medium UCH-L1, GFAP, CK-MB, β-hCG, TSH, homocysteine, and/or free T4 concentration level, for example, about 25000 μg/mL, about 40000 μg/mL, about 45000 μg/mL, about 50000 μg/mL, about 55000 μg/mL, about 60000 μg/mL, about 75000 μg/mL or about 100000 μg/mL; and/or a low UCH-L1, GFAP, CK-MB, β-hCG, TSH, homocysteine, and/or free T4 concentration level, for example, about 1 μg/mL, about 5 μg/mL, about 10 μg/mL, about 12.5 pg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, or about 100 μg/mL.

Any antibodies, which are provided in the kit, such as recombinant antibodies specific for UCH-L1, GFAP, CK-MB, β-hCG, TSH, homocysteine, and/or free T4, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes (e.g., UCH-L1, GFAP, CK-MB, β-hCG, TSH, homocysteine, and/or free T4) or reagents for detecting the analyte (e.g., UCH-L1, GFAP, CK-MB, β-hCG, TSH, homocysteine, and/or free T4). The antibodies, calibrators, and/or controls can be provided in separate containers or pre-dispensed into an appropriate assay format, for example, into microtiter plates,

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays,

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution. If desired, the kit can contain a solid phase, such as a magnetic particle, bead, test tube, microtiter plate, cuvette, membrane, scaffolding molecule, film, filter paper, disc, or chip.

If desired, the kit can further comprise one or more components, alone or in further combination with instructions, for assaying the test sample for another analyte, which can be a biomarker, such as a biomarker of traumatic brain injury or disorder.

The present disclosure has multiple aspects, illustrated by the following non-limiting examples.

### EXAMPLES

### Example 1

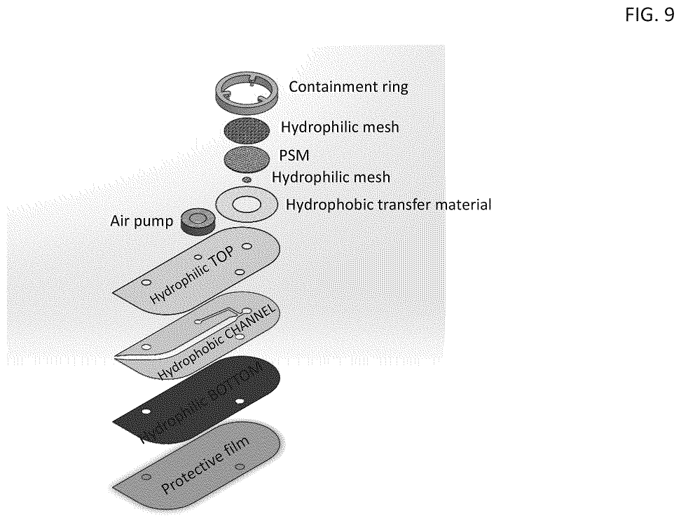

FIG. 9 shows an embodiment of an apparatus that can be used as a plasma separation device in the methods and systems described herein. The apparatus comprises a hydrophilic top layer, a hydrophobic layer with a microchannel having a first and second end, a hydrophilic bottom layer and a protection film. The hydrophilic top layer is adherent to the hydrophobic layer and the protection film is adherent to the bottom hydrophilic layer which is adherent to the hydrophobic layer. The top layer comprises a sample inlet. The sample inlet is surrounded by a circular hydrophobic transfer tape. On top of the hydrophobic transfer tape is a plasma separation membrane (PSM) which is flanked on both its top and bottom surfaces with a hydrophilic mesh. The hydrophilic mesh positioned above or on top of the PSM assists in spreading the sample. The hydrophilic mesh positioned beneath or below the PSM assists facilitating the processing of the blood sample or blood product to the hydrophobic layer. A containment ring, to assist in containing the sample encompasses the PSM, the hydrophilic meshes and the transfer tape. The hydrophobic layer contains an opening (e.g., first opening) directly underneath the sample inlet. The opening on the hydrophobic layer is connected to a first end of the microchannel. Plasma flows from the opening (e.g., first opening) at the first towards the second opening at the second (e.g., opposite) end. The apparatus also contains an air pump for circulating air through the device.

As illustrated in FIG. 9, the apparatus described herein is constructed by stacking various layers and optionally, films on top of one another other. The apparatus is not constructed by perforating or puncturing a substrate (such as an intermediate substrate) or layer and then then including one or more filters or layers in the perforation or puncture to filter the whole blood sample or whole blood product (In alternative aspects not shown in FIG. 9, the apparatus can be constructed by perforating or puncturing a substrate and then including one or more filters or layers in the perforation or puncture).

### Example 2

Blood samples were obtained from 6 donors over 4 different test days. Each blood sample was manipulated to provide several different samples. Specifically, the samples were:
1. Tested without any manipulation.
2. Augmented with native GFAP and UCH-L1 antigen.
3. Spiked with recombinant GFAP and UCH-L1 antigen.

The samples were tested at nominal hematocrit (37-45% Packed Cell Volume (PCV)) and modified hematocrit (low: 30, 33% PCV; high: 44, 50, 52, 54 and 55% PCV)

The samples were drawn into 6 mL K2-EDTA tubes and the plasma was separated by:
1. Centrifugation; or
2. Using a pre-evacuated tube having an inlet and outlet end. The tube had a blood holding chamber at the inlet end and a serum holding chamber at the outlet end. The tube also had a filter assembly between the blood holding chamber at the inlet end and the serum holding chamber at the outlet end. An example of such a device that can be used is described in U.S. Pat. No. 9,427,707.

Once plasma was obtained it was placed in the sample well of a cartridge and tested in a point-of-care device, such as the i-STAT® device of Abbott Laboratories (Abbott Park, IL).

The results confirmed that both native and recombinant GFAP and UCH-L1 could be detected by the point-of-care device using plasma produced using the pre-evacuated tube.

### Example 3

This study was designed to compare the levels of GFAP and UCH-L1 in capillary and venous blood samples obtained from normal subjects. Five (5) subjects were enrolled in this study. Each subject experienced two (2) blood draws. The first draw was a venous blood draw performed by a phlebotomist using routine venous blood collection techniques known in the art. The second blood draw was a capillary blood draw and was performed using the TAP II sample collection device which is commercially available from YourBio Health (Medford, MA), pursuant to the manufacturer's instructions. The TAP II sample collection device is described in WO 2020/223710, the contents of which are herein incorporated by reference.

A portion of each of the venous and capillary samples obtained from every subject was centrifuged to obtain plasma. For each subject, venous whole blood and plasma samples and capillary whole blood and plasma samples were tested.

The venous whole blood and plasma samples and the capillary whole blood and plasma samples for each of the five (5) of the subjects were tested on the commercially available Abbott Laboratories iStat® TBI Plasma test (Abbott Laboratories, Abbott Park, IL).

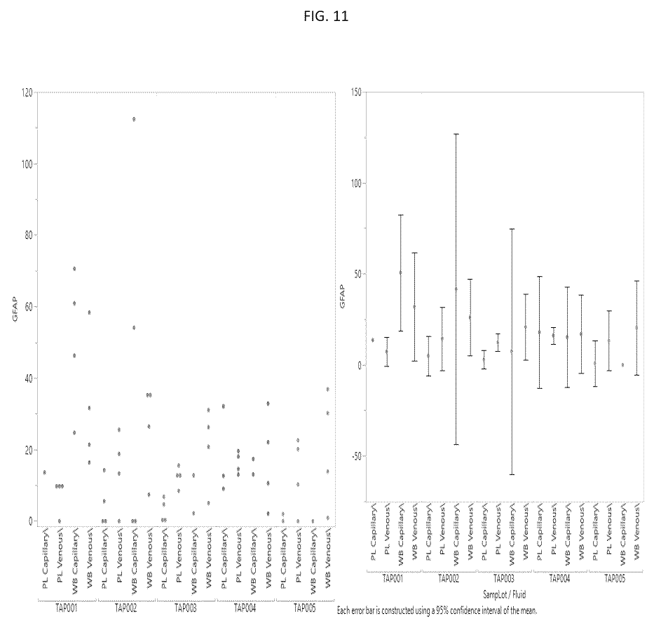

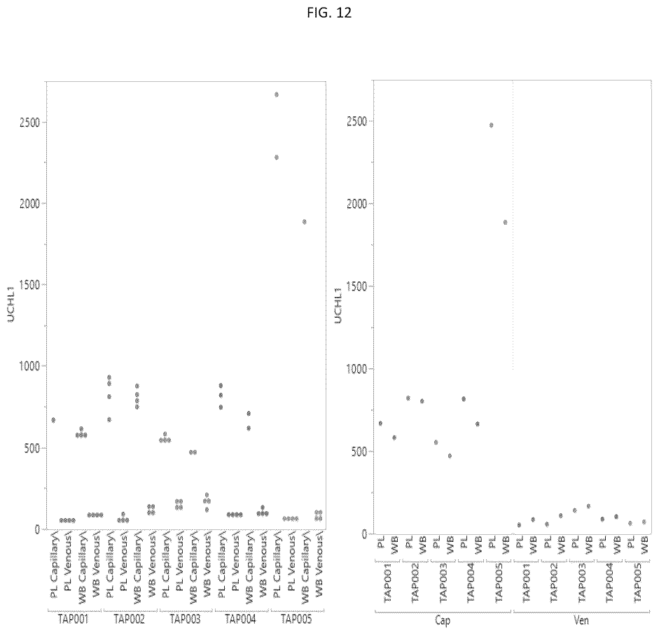

As shown in FIG. 11, the levels of GFAP in the venous whole blood and plasma samples read similar to the levels of GFAP in the capillary whole blood and plasma samples. As shown in FIG. 12, the levels of UCH-L1 in the capillary whole blood and plasma samples were elevated (e.g., about 3 to about 39 times higher) compared to the venous whole blood and plasma samples. While not wishing to be bound by any theory, the elevated levels of UCH-L1 in the capillary whole blood and plasma samples may indicate that UCH-L1 has a role in angiogenesis, namely, the formation of new blood vessels.

### Example 4

This study was designed to compare the levels of GFAP and UCH-L1 in capillary and venous plasma samples obtained from normal subjects. Plasma samples were collected from ten (10) subjects. Each subject experienced two (2) blood draws. The first draw was a venous blood draw performed by a phlebotomist using routine venous blood collection techniques known in the art. The second blood draw was a capillary blood draw and was performed using the TAP II sample collection device which is commercially available from YourBio Health (Medford, MA), pursuant to the manufacturer's instructions.

The venous and capillary blood samples obtained from every subject were used to obtain plasma using the plasma separation device in FIG. 9. For each subject, venous plasma samples and capillary plasma samples were tested.

The venous plasma samples and the capillary plasma samples for each of the ten (10) subjects were tested on the commercially available Abbott Laboratories iStat® TBI Plasma test (Abbott Laboratories, Abbott Park, IL).

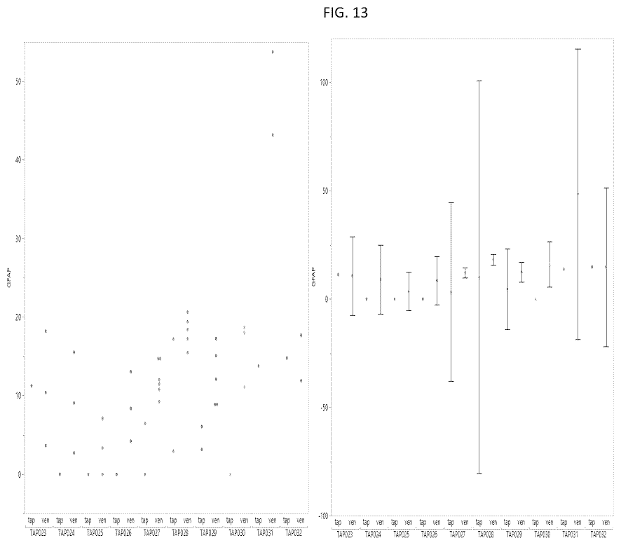

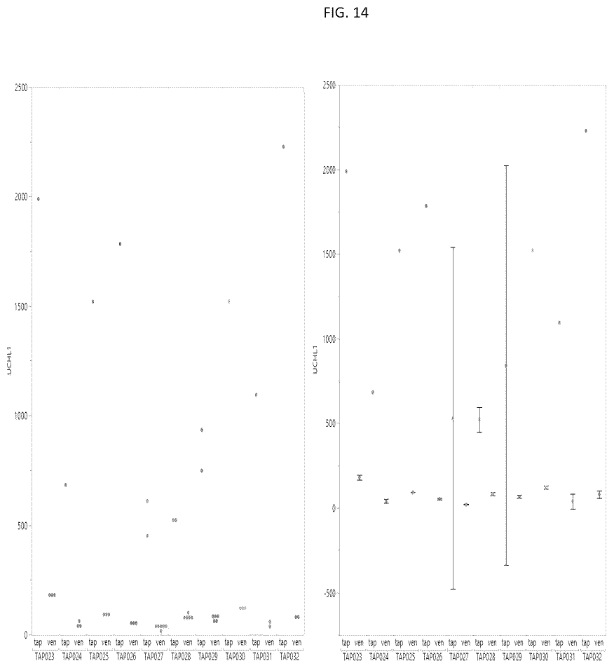

As shown in FIG. 13, the levels of GFAP in the venous plasma samples read similar to the levels of GFAP in the capillary plasma samples. As shown in FIG. 14, the levels of UCH-L1 in the capillary plasma samples were elevated compared to the venous plasma samples in an amount similar to those described in Example 3.

### Example 5

This study was designed to compare the levels of CK-MB in capillary and venous blood samples obtained from normal subjects. Six (6) subjects were enrolled in this study. Each subject experienced two (2) blood draws. The first draw was a venous blood draw performed by a phlebotomist using routine venous blood collection techniques known in the art. The second blood draw was a capillary blood draw and was performed using the TAP II sample collection device which is commercially available from YourBio Health (Medford, MA), pursuant to the manufacturer's instructions. The TAP II sample collection device is described in WO 2020/223710, the contents of which are herein incorporated by reference.

The venous and capillary samples obtained from three (3) of the subjects were used to obtain plasma using the plasma separation device in FIG. 9. For three (3) subjects, venous and capillary whole blood was tested. For the remaining three (3) subjects, venous and capillary plasma was tested.

The venous whole blood and plasma samples and the capillary whole blood and plasma samples for each of the subjects were tested on the commercially available Abbott Laboratories iSTAT® CK-MB test (Abbott Laboratories, Abbott Park, IL). The test was run using investigational/engineering software to allow the test to run on the i-STAT Alinity Instrument.

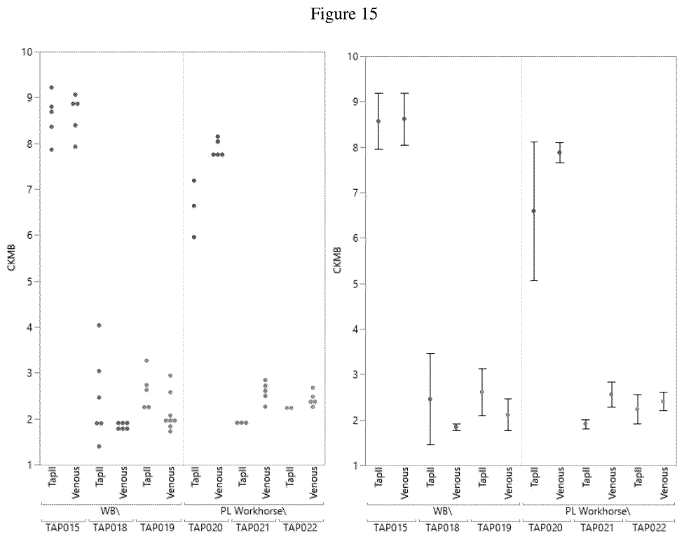

As shown in FIG. 15, the levels of CK-MB in the venous whole blood and plasma samples read similarly to the levels of CK-MB in the capillary whole blood and plasma samples. (e.g., exhibited about a 1.0 to about 0.88 (1:0.88) correlation (e.g., a correlation or conversion factor) between venous whole blood and plasma to capillary whole blood and plasma or exhibited about a 0.88 to about 1.0 (0.88:1:0) correlation (e.g., correlation or conversion factor) between capillary whole blood and plasma to venous whole blood and plasma)).

### Example 6

This study was designed to compare the levels of total β-hCG in capillary and venous blood samples obtained from normal subjects. Six (6) subjects were enrolled in this study. Each subject experienced two (2) blood draws. The first draw was a venous blood draw performed by a phlebotomist using routine venous blood collection techniques known in the art. The second blood draw was a capillary blood draw and was performed using the TAP II sample collection device which is commercially available from YourBio Health (Medford, MA), pursuant to the manufacturer's instructions.

The venous and capillary samples obtained from three (3) of the subjects were used to obtain plasma using the plasma separation device in FIG. 9. For three (3) subjects, venous and capillary whole blood was tested. For the remaining three (3) subjects, venous and capillary plasma was tested.

The venous whole blood and plasma samples and the capillary whole blood and plasma samples for each of the subjects were tested on the commercially available Abbott Laboratories iSTAT® Total β-hCG test (Abbott Laboratories, Abbott Park, IL). The test was run using investigational/engineering software to allow the test to run on the i-STAT Alinity Instrument.

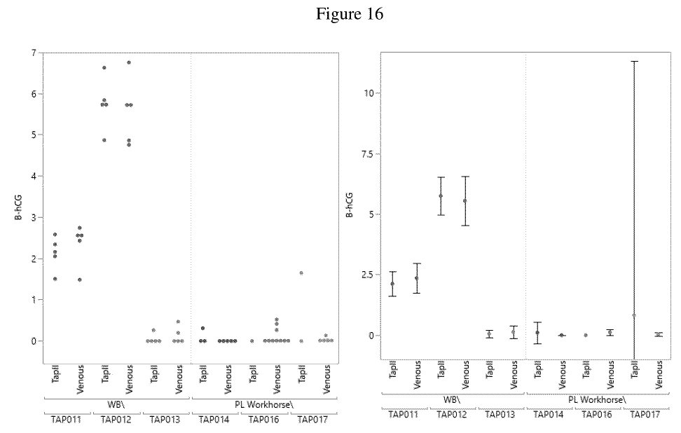

As shown in FIG. 16, the levels of β-hCG in the venous whole blood and plasma samples read similar to the levels of β-hCG in the capillary whole blood and plasma samples (e.g., exhibited about a 1.0 to about 1.0 (1:1) correlation between capillary blood and plasma to venous whole blood and plasma).

### Example 7

This study was designed to compare the levels of TSH, free T4, and homocysteine in capillary and venous blood samples obtained from normal subjects. Six (6) subjects were enrolled in this study. Each subject experienced two (2) blood draws. The first draw was a venous blood draw performed by a phlebotomist using routine venous blood collection techniques known in the art. The second blood draw was a capillary blood draw and was performed using the TAP II sample collection device which is commercially available from YourBio Health (Medford, MA), pursuant to the manufacturer's instructions.

The venous whole blood and plasma samples and the capillary whole blood and plasma samples for each of the subjects were tested on the commercially available Abbott Laboratories ARCHITECT® and/or Alinity (Abbott Laboratories, Abbott Park, IL) instruments using the Abbott ARCHITECT® and/or Alinity TSH, free T4 and homocysteine assays. The tests were run using engineering software to allow the test to run on ARCHITECT® and/or Alinity Instrument. The results are shown in Table A:

TABLE A

| | | | | | % |
|---|---|---|---|---|---|
| | | | | Differ- | Differ- |
| | | | | ence | ence |
| | | | | Between | Between |
| Estimated | ARCHITECT/ | | | Venous | Venous |
| Volume | Alinity | Capillary | Venous | and | and |
| (mL) | Assay | RESULT | RESULT | TAP II | TAP II |
| 0.200 | Free T4 | 1.08 | 1.11 | −0.03 | −2.7% |
| 0.200 | Free T4 | 0.95 | 0.99 | −0.04 | −4.0% |
| 0.200 | Free T4 | 1.06 | 0.99 | 0.07 | 7.1% |
| 0.200 | Free T4 | 1.05 | 1.09 | −0.04 | −3.7% |
| 0.200 | Free T4 | 0.77 | 0.83 | −0.06 | −7.2% |
| 0.200 | Free T4 | 1.14 | 1.1 | 0.04 | 3.6% |
| 0.300 | TSH | 2.2283 | 2.8121 | −0.58 | −20.8% |
| 0.500 | TSH | 0.8118 | 0.7992 | 0.01 | 1.6% |
| 0.400 | TSH | 1.2143 | 1.2487 | −0.03 | −2.8% |
| 0.500 | TSH | 0.879 | 0.9555 | −0.08 | −8.0% |
| 0.400 | TSH | 0.8867 | 0.9151 | −0.03 | −3.1% |
| 0.400 | TSH | 3.9087 | 4.3909 | −0.48 | −11.0% |
| 0.200 | Homocysteine | 10.3 | 9.47 | 0.83 | 8.8% |
| 0.400 | Homocysteine | 10.09 | 9.84 | 0.25 | 2.5% |
| 0.200 | Homocysteine | 9.72 | 9.13 | 0.59 | 6.5% |
| 0.400 | Homocysteine | 11.81 | 11.01 | 0.80 | 7.3% |
| 0.400 | Homocysteine | 19.62 | 19.64 | −0.02 | −0.1% |
| 0.400 | Homocysteine | 12.41 | 11.7 | 0.71 | 6.1% |

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the non-limiting examples described herein.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

For reasons of completeness, various aspects of the disclosure are set out in the following numbered clauses:

Clause 1. A method comprising:
performing at least one assay for ubiquitin carboxy-terminal hydrolase L1 (UCH-L1), glial fibrillary acidic protein (GFAP), or a combination thereof on a capillary blood sample obtained from a subject to determine an amount of UCH-L1, GFAP, or a combination thereof; and
communicating the amount of UCH-L1, GFAP, or combination thereof determined in the sample in about 4 to about 40 minutes from the time the sample is collected,
wherein the sample is collected (1) in a decentralized setting; (2) without the use of a syringe, standard needle, or combination thereof; (3) by a user not trained in collecting blood samples from a subject; (4) by a robot; (5) by a self-or other-administered blood collection device; or (6) any combination of (1)-(5).

Clause 2. The method of clause 1, wherein the assay comprises contacting the sample with (a) an anti-UCH-L1 antibody that binds to UCH-L1 to determine the amount of UCH-L1 in the sample; (b) an anti-GFAP antibody that binds to GFAP to determine the amount of GFAP in the sample; or (c) a combination of (a) and (b).

Clause 3. The method of clause 1 or clause 2, wherein the sample is processed prior to performing the assay.

Clause 4. The method of clauses 1-3, wherein the method is used to aid in a diagnosis and evaluation of a subject that has sustained or may have sustained an injury to the head.

Clause 5. The method of clauses 1-4, wherein at least one assay is performed for UCH-L1 and at least one assay is performed for GFAP.

Clause 6. The method of clause 3, wherein the sample is whole blood and is processed into serum or plasma.

Clause 7. The method of clauses 1-6, wherein the amount of UCH-L1, GFAP, and UCH-L1 and UCH-L1 and GFAP is communicated in: (a) about 40 minutes from the time the sample is collected; (b) about 30 minutes from the time the sample is collected; (c) about 20 minutes from the time the sample is collected; (d) about 18 minutes from the time the sample is collected; (e) about 15 minutes from the time the sample is; (f) about 5 minutes from the time of the injury or suspected injury; or (g) about 4 minutes from the time the sample is collected.

Clause 8. The method of clauses 1-7, wherein the assay is an analog assay, a digital assay, or a combination of an analog assay or a digital assay.

Clause 9. The method of clauses 1-8, wherein the sample is collected using a microsampling device.

Clause 10. The method of clauses 3-9, wherein the sample is processed using a plasma separation device.

Clause 11. The method of clause 10, wherein the plasma separation device is operably linked to the microsampling device.

Clause 12. The method of clause 10, wherein the microsampling device comprises a plasma separation device.

Clause 13. The method of clauses 10-12, wherein the plasma separation device comprises a filter, a membrane, synthetic paper, or any combinations thereof.

Clause 14. The method of clauses 1-13, wherein the instrument is a point-of-care device.

Clause 15. The method of clause 14, wherein the point-of-care device comprises a cartridge.

Clause 16. The method of clause 15, wherein the plasma separation device is operably linked to the cartridge.

Clause 17. The method of clauses 1-13, wherein the instrument is a higher throughput assay analyzer.

Clause 18. The method of clauses 1-17, wherein the amount of UCH-L1, GFAP or UCH-L1 and GFAP is communicated by an instrument.

Clause 19. The method of clause 18, wherein the amount of the UCH-L1, GFAP, or UCH-L1 and GFAP in the sample is communicated in a document and/or spreadsheet, on a mobile device, on a computer, on a website, in an e-mail, or any combination thereof.

Clause 20. The method of clause 18 or clause 19, wherein the amount of UCH-L1, GFAP, or UCH-L1 and GFAP is communicated by displaying on an instrument.

Clause 21. The method of clauses 1-20, wherein the subject is a human.

Clause 22. A system comprising:
a microsampling device to collect a capillary blood sample from a subject;
a reaction vessel that receives the capillary blood sample and comprises an assay for ubiquitin carboxy-terminal hydrolase L1 (UCH-L1), glial fibrillary acidic protein (GFAP), or a combination thereof; and
an instrument to analyze the reaction vessel to provide an amount of UCH-L1, GFAP, or UCH-L1 and GFAP in the sample.

Clause 23. The system of clause 22, wherein the system further comprises a plasma separation device to create a processed capillary blood sample.

Clause 24. The system of clause 22 or clause 23, wherein the reaction vessel comprises an aperture.

Clause 25. The system of clauses 22-24, wherein the microsampling device includes a housing, a microneedle, a lancet, a microlancet, a blade, a microblade, a microscrew, or any combination thereof coupled to the housing, and a receptacle coupled to the housing; wherein the capillary blood sample is collected in the receptacle.

Clause 26. The system of clause 25, wherein the receptacle is removably coupled to the housing.

Clause 27. The system of clauses 25-26, wherein the microsampling device further comprises a cap coupled to the receptacle, wherein the cap seals the capillary blood sample within the receptacle.

Clause 28. The system of clauses 25-27, wherein the microsampling device further comprises an actuator movable relative to the housing.

Clause 29. The system of claim 24, wherein the plasma separation device is in fluid communication with the aperture at any point along the reaction vessel.

Clause 30. The system of claim 29, wherein the plasma separation device is placed in fluid communication with the aperture at one end, on a side, or in the middle of the reaction vessel.

Clause 31. The system of claim 30, wherein the plasma separation device is placed in fluid communication with the aperture at an end or side of the reaction vessel at an angle.

Clause 32. The system of clauses 22-28, further comprising a transfer tube.

Clause 33. The system of clause 32, wherein the transfer tube comprises a cap or a stopper.

Clause 34. The system of clauses 23-33, wherein the plasma separation device includes an inlet to receive the capillary blood sample from the microsampling device and an outlet through which the processed capillary blood sample leaves the plasma separation device.

Clause 35. The system of clause 34, wherein the outlet of the plasma separation device is in fluid communication with the aperture of the reaction vessel.

Clause 36. The system of clause 34, wherein the outlet of the plasma separation device is in fluid communication with the cap or stopper of the transfer tube.

Clause 37. The system of clause 36, wherein the cap or stopper of the transfer tube is in fluid communication with the aperture of the reaction vessel.

Clause 38. The system of clauses 25-37, wherein the receptacle is squeezed to force the capillary blood sample through the plasma separation device and into the reaction vessel or transfer tube.

Clause 39. The system of clauses 25-37, wherein the receptacle includes a plunger to force the capillary blood sample through the plasma separation device and into the reaction vessel or transfer tube.

Clause 40. The system of clauses 23-33, wherein the plasma separation device is integrated within the receptacle.

Clause 41. The system of clause 40, wherein the receptacle is a reaction vessel.

Clause 42. The system of clauses 23-33, wherein the plasma separation device is integrated within the reaction vessel.

Clause 43. The system of clauses 23-33, wherein the plasma separation device is integrated into the transfer tube.

Clause 44. The system of clauses 23-43, wherein the plasma separation device includes a filter, a membrane, a synthetic paper, or any combinations thereof.

Clause 45. The system of clauses 22-44, wherein the amount of UCH-L1, GFAP, or UCH-L1 and GFAP is determined in about 4 to about 40 minutes from the time the sample is collected.

Clause 46. The system of clause 45, wherein the amount of UCH-L1, GFAP or UCH-L1 and GFAP is communicated by the instrument.

Clause 47. The system of clause 45, wherein the amount of the UCH-L1, GFAP, or UCH-L1 and GFAP is communicated in a document and/or spreadsheet, on a mobile device, on a computer, on a website, in an e-mail, or any combination thereof.

Clause 48. The system of clause 45 or clause 47, wherein the amount of UCH-L1, GFAP, or UCH-L1 and GFAP is communicated by displaying on the instrument.

Clause 49. The system of clauses 22-48, wherein at least a portion of the system is usable in a decentralized setting.

Clause 50. The method of clause 3, wherein the sample is processed by centrifugation.

Clause 51. The method of clause 4, wherein the subject is diagnosed as having a traumatic brain injury.

Clause 52. The method of clause 51, wherein the subject is treated for the traumatic brain injury.

Clause 53. The method of clause 10, wherein the plasma separation device is an apparatus comprising:
a hydrophobic layer comprising at least one microchannel having a first and second end and which defines a path for capillary fluid flow; and
a top layer flanking the hydrophobic layer, wherein a surface of the top layer facing the hydrophobic layer is hydrophilic, wherein the sample comprises blood or blood products.

Clause 54. The method of clause 53, wherein the apparatus further comprises a bottom layer flanking the hydrophobic layer, wherein a surface of the bottom layer facing the hydrophobic layer is hydrophilic.

Clause 55. The method of clause 53 or clause 54, wherein the top layer, hydrophobic layer, bottom layer, or any combination thereof are adherent to each other.

Clause 56. The method of clauses 53-55, wherein the at least one microchannel extends longitudinally along a portion of the hydrophobic layer to an opening at a second end.

Clause 57. The method of clauses 53-56 wherein the at least one microchannel is less than about 80 mm in length.

Clause 58. The method of clauses 53-57, wherein the at least one microchannel is less than about 5 mm wide.

Clause 59. The method of clauses 53-58, wherein the top layer, bottom layer or top and bottom layers are entirely hydrophilic.

Clause 60. The method of clauses 53-59, wherein a composition of the entirety of the top layer, bottom layer, or both the top and bottom layers each comprise same or different materials.

Clause 61. The method of clauses 53-60, wherein the hydrophobic layer, top layer, bottom layer or any combination thereof have a combined thickness of about 100 to about 600 microns.

Clause 62. The method of clauses 53-61, wherein the hydrophobic layer has thickness of about 50 to about 200 microns.

Clause 63. The method of clauses 53-62, wherein each of the top layer, bottom layer, or top and bottom layers have a thickness of about 50 to about 200 microns.

Clause 64. The method of clauses 53-63, wherein the top layer comprises a sample inlet.

Clause 65. The method of clause 64, wherein the hydrophobic layer and optionally, the bottom layer, comprise an opening below the sample inlet.

Clause 66. The method of clause 64 or clause 65, wherein the sample inlet comprises a separation membrane.

Clause 67. The method of clause 66, wherein the separation membrane is a plasma separation membrane.

Clause 68. The method of clause 66 or clause 67, further comprising a hydrophilic mesh or hydrophilic film positioned above the separation membrane, below the separation membrane, or both above and below the separation membrane.

Clause 69. The method of clauses 65-68, wherein the opening in the hydrophobic layer is connected to the first end of the at least one microchannel.

Clause 70. The method of clauses 53-69, further comprising an agglutinating agent.

Clause 71. The method of clause 70, wherein the agglutinating agent comprises lectin, Merquat-100, Concanavalin A, DEAE-Dextran, poly-L-lysine, polyvinylpyrrolidone, poly(2-(dimethylamino)ethylmethacrylate), or any combinations thereof.

Clause 72. The method of clauses 53-71, wherein the top layer, hydrophobic layer, bottom layer, hydrophilic mesh or hydrophilic film, separation membrane, or any combination thereof is either ubiquitous for any analyte or specific for an analyte or class of analytes.

Clause 73. The method of clauses 53-55, wherein the at least one microchannel extends along a width of a portion of the hydrophobic layer to an opening at a second end.

Clause 74. The method of clause 10, wherein the plasma separation device is an apparatus comprising:
a container or tube having an inlet and outlet end;
a blood holding chamber at the inlet end of the container or tube;
a detachable serum holding chamber at the outlet end of the container or tube;
a material located within the container between the blood holding chamber and the serum holding chamber for separating components of whole blood based on size.

Clause 75. The method of clause 74, wherein the material is glass or porous beads, membranes, a filter, glass or other fiber materials, or any combination thereof.

Clause 76. The method of clause 74 or 75, wherein the filter permits the passage of particles or molecules smaller than about 0.7 microns, about 0.6 microns, about 0.5 microns, about 0.4 microns, or about 0.3 microns.

Clause 77. The method of clauses 74-76, wherein a pressure differential between the blood holding chamber and the serum holding chamber allows for whole blood to travel from the blood holding chamber through the filter to produce serum and/or plasma which is collected in the serum holding chamber.

Clause 78. The system of clauses 23-33, wherein the plasma separation device comprises an apparatus having:
a) a hydrophobic layer comprising at least one microchannel having a first and second end and which defines a path for capillary fluid flow; and a top layer flanking the hydrophobic layer, wherein a surface of the top layer facing the hydrophobic layer is hydrophilic; or
b) a pre-evacuated container or tube having an inlet and outlet end; a blood holding chamber at the inlet end of the container or tube; a detachable serum holding chamber at the outlet end of the container or tube; a filter located within the container between the blood holding chamber and the serum holding chamber.

Clause 79. The system of clause 78, wherein the apparatus in a) further comprises a bottom layer flanking the hydrophobic layer, wherein a surface of the bottom layer facing the hydrophobic layer is hydrophilic.

Clause 80. The system of clause 78 or clause 79, wherein the top layer, hydrophobic layer, bottom layer, or any combination thereof are adherent to each other.

Clause 81. The system of clauses 78-80, wherein the at least one microchannel extends longitudinally along a portion of the hydrophobic layer to an opening at a second end.

Clause 82. The system of clauses 78-81 wherein the at least one microchannel is less than about 80 mm in length.

Clause 83. The system of clauses 78-82, wherein the at least one microchannel is less than about 5 mm wide.

Clause 84. The system of clauses 78-83, wherein the top layer, bottom layer or top and bottom layers are entirely hydrophilic.

Clause 85. The system of clauses 78-84, wherein a composition of the entirety of the top layer, bottom layer, or both the top and bottom layers each comprise same or different materials.

Clause 86. The system of clauses 78-85, wherein the hydrophobic layer, top layer, bottom layer or any combination thereof have a combined thickness of about 100 to about 600 microns.

Clause 87. The system of clauses 78-86, wherein the hydrophobic layer has thickness of about 50 to about 200 microns.

Clause 88. The system of clauses 78-87, wherein each of the top layer, bottom layer, or top and bottom layers have a thickness of about 50 to about 200 microns.

Clause 89. The system of clauses 78-88, wherein the top layer comprises a sample inlet.

Clause 90. The system of clause 89, wherein the hydrophobic layer and optionally, the bottom layer, comprise an opening below the sample inlet.

Clause 91. The system of clause 89 or clause 90, wherein the sample inlet comprises a separation membrane.

Clause 92. The system of clause 91, wherein the separation membrane is a plasma separation membrane.

Clause 93. The system of clause 91 or clause 92, further comprising a hydrophilic mesh or hydrophilic film positioned above the separation membrane, below the separation membrane, or both above and below the separation membrane.

Clause 94. The system of clauses 90-93, wherein the opening in the hydrophobic layer is connected to the first end of the at least one microchannel.

Clause 95. The system of clauses 78-94, further comprising an agglutinating agent.

Clause 96. The system of clause 95, wherein the agglutinating agent comprises lectin, Merquat-100, Concanavalin A, DEAE-Dextran, poly-L-lysine, polyvinylpyrrolidone, poly(2-(dimethylamino)ethylmethacrylate), or any combinations thereof.

Clause 97. The system of clause 78, wherein the filter permits the passage of particles or molecules smaller than about 0.7 microns, about 0.6 microns, about 0.5 microns, about 0.4 microns, or about 0.3 microns.

Clause 98. The system of clause 78 or clause 97, wherein a pressure differential between the blood holding chamber and the serum holding chamber allows for whole blood to travel from the blood holding chamber through the filter to produce serum and/or plasma which is collected in the serum holding chamber.

Clause 99. A method comprising:
performing at least one assay for ubiquitin carboxy-terminal hydrolase L1 (UCH-L1), glial fibrillary acidic protein (GFAP), or a combination thereof on a blood sample obtained from a subject to determine an amount of UCH-L1, GFAP, or a combination thereof; and
communicating the amount of UCH-L1, GFAP, or combination thereof determined in the sample,
wherein the sample is collected with the use of a syringe, standard needle, or combination thereof; and
further wherein the sample is processed prior to performing the assay with a plasma separation device comprising an apparatus having:
a hydrophobic layer comprising at least one microchannel having a first and second end and which defines a path for capillary fluid flow; and a top layer flanking the hydrophobic layer, wherein a surface of the top layer facing the hydrophobic layer is hydrophilic; or
a pre-evacuated container or tube having an inlet and outlet end;
a blood holding chamber at the inlet end of the container or tube;
a detachable serum holding chamber at the outlet end of the container or tube;
a filter located within the container between the blood holding chamber and the serum holding chamber.

Clause 100. The method of clause 99, wherein the blood sample is a venous blood sample or a capillary blood sample.

Clause 101. The method of clause 99 or clause 100, wherein the sample is collected in a decentralized or a centralized setting.

Clause 102. The method of clauses 99-101, wherein the apparatus in a) further comprises a bottom layer flanking the hydrophobic layer, wherein a surface of the bottom layer facing the hydrophobic layer is hydrophilic.

Clause 103. The method of clause 99 or clause 101, wherein the top layer, hydrophobic layer, bottom layer, or any combination thereof are adherent to each other.

Clause 104. The method of clauses 99-101, wherein the at least one microchannel extends longitudinally along a portion of the hydrophobic layer to an opening at a second end.

Clause 105. The method of clauses 99-104, wherein the at least one microchannel is less than about 80 mm in length.

Clause 106. The method of clauses 99-105, wherein the at least one microchannel is less than about 5 mm wide.

Clause 107. The method of clauses 99-106, wherein the top layer, bottom layer or top and bottom layers are entirely hydrophilic.

Clause 108. The method of clauses 99-107, wherein a composition of the entirety of the top layer, bottom layer, or both the top and bottom layers each comprise same or different materials.

Clause 109. The method of clauses 99-108, wherein the hydrophobic layer, top layer, bottom layer or any combination thereof have a combined thickness of about 100 to about 600 microns.

Clause 110. The method of clauses 99-109, wherein the hydrophobic layer has thickness of about 50 to about 200 microns.

Clause 111. The method of clauses 99-110, wherein each of the top layer, bottom layer, or top and bottom layers have a thickness of about 50 to about 200 microns.

Clause 112. The method of clauses 99-111, wherein the top layer comprises a sample inlet.

Clause 113. The method of clause 112, wherein the hydrophobic layer and optionally, the bottom layer, comprise an opening below the sample inlet.

Clause 114. The method of clause 112 or clause 113, wherein the sample inlet comprises a separation membrane.

Clause 115. The method of clause 114, wherein the separation membrane is a plasma separation membrane.

Clause 116. The method of clause 114 or clause 115, further comprising a hydrophilic mesh or hydrophilic film positioned above the separation membrane, below the separation membrane, or both above and below the separation membrane.

Clause 117. The method of clauses 111-116, wherein the opening in the hydrophobic layer is connected to the first end of the at least one microchannel.

Clause 118. The method of clauses 99-117, further comprising an agglutinating agent.

Clause 119. The method of clause 118, wherein the agglutinating agent comprises lectin, Merquat-100, Concanavalin A, DEAE-Dextran, poly-L-lysine, polyvinylpyrrolidone, poly(2-(dimethylamino)ethylmethacrylate), or any combinations thereof.

Clause 120. The method of clause 99, wherein the filter permits the passage of particles or molecules smaller than about 0.7 microns, about 0.6 microns, about 0.5 microns, about 0.4 microns, or about 0.3 microns.

Clause 121. The method of clause 99 or clause 120, wherein a pressure differential between the blood holding chamber and the serum holding chamber allows for whole blood to travel from the blood holding chamber through the filter to produce serum and/or plasma which is collected in the serum holding chamber.

Clause 122. A system comprising:
a plasma separation device to process a whole blood sample obtained from a subject into serum and/or plasma;
a reaction vessel that receives the serum and/or plasma from the subject and comprises an assay for ubiquitin carboxy-terminal hydrolase L1 (UCH-L1), glial fibrillary acidic protein (GFAP), or a combination thereof; and
an instrument to analyze the reaction vessel to provide an amount of UCH-L1, GFAP, or UCH-L1 and GFAP in the sample,
wherein the plasma separation device comprises an apparatus having:
a hydrophobic layer comprising at least one microchannel having a first and second end and which defines a path for capillary fluid flow; and a top layer flanking the hydrophobic layer, wherein a surface of the top layer facing the hydrophobic layer is hydrophilic; or
a pre-evacuated container or tube having an inlet and outlet end;
a blood holding chamber at the inlet end of the container or tube;
a detachable serum holding chamber at the outlet end of the container or tube;
a filter located within the container between the blood holding chamber and the serum holding chamber.

Clause 123. The system of clause 122, wherein the blood sample is a venous blood sample or a capillary blood sample.

Clause 124. The system of clause 122 or clause 123, wherein the sample is collected in a decentralized or a centralized setting.

Clause 125. The system of clause 124, wherein the apparatus in a) further comprises a bottom layer flanking the hydrophobic layer, wherein a surface of the bottom layer facing the hydrophobic layer is hydrophilic.

Clause 126. The system of clause 122 or clause 124, wherein the top layer, hydrophobic layer, bottom layer, or any combination thereof are adherent to each other.

Clause 127. The system of clauses 122-126, wherein the at least one microchannel extends longitudinally along a portion of the hydrophobic layer to an opening at a second end.

Clause 128. The system of clauses 122-127, wherein the at least one microchannel is less than about 80 mm in length.

Clause 129. The system of clauses 122-127, wherein the at least one microchannel is less than about 5 mm wide.

Clause 130. The system of clauses 122-127, wherein the top layer, bottom layer or top and bottom layers are entirely hydrophilic.

Clause 131. The system of clauses 122-130, wherein a composition of the entirety of the top layer, bottom layer, or both the top and bottom layers each comprise same or different materials.

Clause 132. The system of clauses 122-131, wherein the hydrophobic layer, top layer, bottom layer or any combination thereof have a combined thickness of about 100 to about 600 microns.

Clause 133. The system of clauses 122-132, wherein the hydrophobic layer has thickness of about 50 to about 200 microns.

Clause 134. The system of clauses 122-133, wherein each of the top layer, bottom layer, or top and bottom layers have a thickness of about 50 to about 200 microns.

Clause 135. The system of clauses 122-134, wherein the top layer comprises a sample inlet.

Clause 136. The system of clause 135, wherein the hydrophobic layer and optionally, the bottom layer, comprise an opening below the sample inlet.

Clause 137. The system of clause 135 or clause 136, wherein the sample inlet comprises a separation membrane.

Clause 138. The system of clause 137, wherein the separation membrane is a plasma separation membrane.

Clause 139. The system of clause 137 or clause 138, further comprising a hydrophilic mesh or hydrophilic film positioned above the separation membrane, below the separation membrane, or both above and below the separation membrane.

Clause 140. The system of clauses 136-139, wherein the opening in the hydrophobic layer is connected to the first end of the at least one microchannel.

Clause 141. The system of clauses 122-140, further comprising an agglutinating agent.

Clause 142. The system of clause 141, wherein the agglutinating agent comprises lectin, Merquat-100, Concanavalin A, DEAE-Dextran, poly-L-lysine, polyvinylpyrrolidone, poly(2-(dimethylamino)ethylmethacrylate), or any combinations thereof.

Clause 143. The system of clause 122, wherein the filter permits the passage of particles or molecules smaller than about 0.7 microns, about 0.6 microns, about 0.5 microns, about 0.4 microns, or about 0.3 microns.

Clause 144. The system of clause 122 or clause 143, wherein a pressure differential between the blood holding chamber and the serum holding chamber allows for whole blood to travel from the blood holding chamber through the filter to produce serum and/or plasma which is collected in the serum holding chamber.

Clause 145. The method of clauses 1-3, 5-10, 14-15, 18, 20-21, 50, and 53-69, wherein the level of UCH-L1 in the capillary blood sample obtained from the subject is about 3 times to about 39 times higher than the level of UCH-L1 in a venous blood sample obtained from the subject.

Clause 146. The system of clauses 22-23, 25 and 78-94, wherein the level of UCH-L1 in the capillary blood sample obtained from the subject is about 3 times to about 39 times higher than the level of UCH-L1 in a venous blood sample obtained from the subject.

Clause 147. A method comprising:
(A) performing at least one assay for (i) ubiquitin carboxy-terminal hydrolase L1 (UCH-L1), glial fibrillary acidic protein (GFAP), or a combination thereof; or (ii) CK-MB, β-hCG, thyroid stimulating hormone (TSH), homocysteine, free thyroxine (free T4), or any combination thereof, on a capillary blood sample obtained from a subject to determine an amount of (i) UCH-L1, GFAP, or a combination thereof; (ii) CK-MB, β-hCG, TSH, homocysteine, free T4, or any combination thereof; or (iii) or any combination of (i) and (ii); and
(B) communicating the amount of (i) UCH-L1, GFAP, or combination thereof; and/or (ii) CK-MB, β-hCG, TSH, homocysteine, free T4, or any combination thereof, determined in the sample
using a point-of-care device, a non-point-of-care device, or lateral flow device,
wherein the sample is collected from a location on the subject other than a digit and (1) in a decentralized setting; (2) without the use of a syringe, standard needle, or combination thereof; (3) by a user not trained in collecting blood samples from a subject; (4) by a robot; (5) by a self-or other-administered blood collection device; or (6) any combination of (1)-(5), and
further wherein: (i) the assay is capable of being performed in less than about 30 minutes; (ii) the amount of (a) UCH-L1, GFAP, or combination thereof; or (B) CK-MB, β-hCG, TSH, homocysteine, free T4, or any combination thereof, determined in the sample is capable of being communicated in less than about 30 minutes from the time the sample is collected; or (iii) a combination of (i) and (ii).

Clause 148. The method of clause 147, wherein the sample is processed prior to performing the assay.

Clause 149. The method of clause 148, wherein the sample is whole blood and is processed into serum or plasma.

Clause 150. The method of clause 149, wherein the sample is processed using a plasma separation device.

Clause 151. The method of clauses 147-150, wherein the sample is collected using a microsampling device.

Clause 152. The method of clause 151, wherein the microsampling device comprises a plasma separation device.

Clause 153. The method of clause 152, wherein the plasma separation device is operably linked to the microsampling device.

Clause 154. The method of clauses 147-153, wherein the method comprises performing at least one assay for UCH-L1, at least one assay for GFAP, or at least one assay for UCH-L1 and at least one assay for GFAP.

Clause 155. The method of clause 154, wherein the assay comprises contacting the sample with (a) an anti-UCH-L1 antibody that binds to UCH-L1 to determine the amount of UCH-L1 in the sample; (b) an anti-GFAP antibody that binds to GFAP to determine the amount of GFAP in the sample; or (c) a combination of (a) and (b).

Clause 156. The method of clauses 154 or 155, wherein when the assay is for: (a) GFAP, the GFAP assay comprises a conversion factor for GFAP in a capillary sample compared to GFAP in a venous sample of about 1.0:1.0; (b) UCH-L1, the UCH-L1 assay comprises a conversion factor for UCH-L1 in a capillary sample compared to UCH-L1 in a venous sample of about 2.5:1.0 to about 1.5:1.0; or (c) a combination of (a) and (b).

Clause 157. The method of clauses 154-156, wherein the method is used to aid in a diagnosis and evaluation of a subject that has sustained or may have sustained an injury to the head.

Clause 158. The method of clauses 154-157, wherein the amount of UCH-L1, GFAP, and UCH-L1 and UCH-L1 and GFAP is communicated in: (a) about 25 minutes from the time the sample is collected; (b) less than about 20 minutes from the time the sample is collected; (c) about 4 to about 20 minutes from the time the sample is collected; (d) about 15 to about 18 minutes from the time the sample is collected; or (e) less than about 18 minutes from the time the sample is collected.

Clause 159. The method of clauses 147-153, wherein the method comprises performing at least one assay for CK-MB, at least one assay for β-hCG, at least one assay for TSH, at least one assay for homocysteine, at least one assay for free T4, or any combination thereof.

Clause 160. The method of clause 159, wherein the assay comprises contacting the sample with (a) an anti-CK-MB antibody that binds to CK-MB to determine the amount of CK-MB in the sample; (b) an anti-β-hCG antibody that binds to β-hCG to determine the amount of β-hCG in the sample; (c) an anti-TSH antibody that binds to TSH to determine the amount of TSH in the sample; (d) an anti-homocysteine antibody that binds to homocysteine to determine the amount of homocysteine in the sample; (e) an anti-free T4 antibody that binds to free T4 to determine the amount of free T4 in the sample; or (f) any combination of (a) to (e).

Clause 161. The method of clauses 159 or 160, wherein when the assay is for: (a) CK-MB, the CK-MB assay comprises a conversion factor for CK-MB in a capillary sample compared to CK-MB in a venous sample of about 0.5:1.0 to about 1:0:1.2; (b) β-hCG, the β-hCG assay comprises a conversion factor for β-hCG in a capillary sample compared to β-hCG in a venous sample of about 0.8:1.0 to about 1.0:1.4; (c) TSH, the TSH assay comprises a conversion factor for TSH in a capillary sample compared to TSH in a venous sample of about 0.75:1.0 to about 1.2:1.0; (d) homocysteine, the homocysteine assay comprises a conversion factor for homocysteine in a capillary sample compared to homocysteine in a venous sample of about 1.2:1.0 to about 0.9:1.0; (e) free T4, the free T4 assay comprises a conversion factor for free T4 in a capillary sample compared to free T4 in a venous sample of about 0.8:1.0 to about 1.2:1.0; (f) any combination of (a) to (e).

Clause 162. The method of clauses 159-161, wherein when the assay is for: (1) CK-MB, the method is used to diagnose myocardial infarction in a subject; (2) β-hCG, the method is used to determine if a subject is pregnant; (3) TSH, the method is used to assess thyroid function in a subject, diagnose thyroid disease in a subject, treat thyroid disease in a subject, or any combinations thereof; (4) homocysteine, the method is used to diagnose hyperhomocysteinemia, homocystinuria, or hyperhomocysteinemia and homocystinuria in a subject, or treat subjects having hyperhomocysteinemia, homocystinuria, or hyperhomocysteinemia and homocystinuria; or (5) free T4, the method is used to assess thyroid function in a subject, diagnose thyroid disease in a subject, treat thyroid disease in a subject, or any combination thereof.

Clause 163. The method of clauses 159-162, wherein the amount of CK-MB, B-hCG, TSH, homocysteine, free T4, or any combination thereof, is communicated in: (a) about 25 minutes from the time the sample is collected; (b) less than about 20 minutes from the time the sample is collected; (c) about 4 to about 20 minutes from the time the sample is collected; (d) about 15 to about 18 minutes from the time the sample is collected; or (e) less than about 18 minutes from the time the sample is collected.

Clause 164. The method of clauses 147-163, wherein the point-of-care device comprises a cartridge.

Clause 165. The method of clauses 147-163, wherein the non-point-of-care device is a higher throughput assay analyzer.

Clause 166. The method of clauses 147-165, wherein the amount of the UCH-L1, GFAP, CK-MB, β-hCG, TSH, homocysteine, free T4, or any combination thereof, is communicated by the point-of-care device or non-point-of-care device in a document and/or spreadsheet, on a mobile device, on a computer, on a website, in an e-mail, or any combination thereof.

Clause 167. The method of clauses 147-165, wherein the amount UCH-L1, GFAP, CK-MB, β-hCG, TSH, homocysteine, free T4, or any combination thereof, is communicated by displaying on the point-of-care device or non-point-of-care device.

Clause 168. The method of clauses 147-167, wherein the assay is an analog assay, a digital assay, or a combination of an analog assay or a digital assay.

Clause 169. The method of clauses 147-168, wherein the subject is a human.

Clause 170. A system comprising:
a microsampling device to collect a capillary blood sample from a subject;
a reaction vessel that receives the capillary blood sample and comprises an assay for (i) ubiquitin carboxy-terminal hydrolase L1 (UCH-L1), glial fibrillary acidic protein (GFAP), or a combination thereof; (ii) CK-MB, β-hCG, thyroid stimulating hormone (TSH), homocysteine, free thyroxine (free T4), or any combination thereof; or (iii) any combination of (i) and (ii); and
an instrument to analyze the reaction vessel to provide an amount of (i) UCH-L1, GFAP, or UCH-L1 and GFAP; and/or (ii) CK-MB, β-hCG, TSH, homocysteine, free T4, or any combination thereof, in the sample.

Clause 171. The system of clause 170, wherein the system further comprises a plasma separation device to create a processed capillary blood sample.

Clause 172. The system of clause 170 or clause 171, wherein the reaction vessel comprises an aperture.

Clause 173. The system of clauses 170-172, wherein the microsampling device includes a housing, a microneedle, a lancet, a microlancet, a blade, a microblade, a microscrew, or any combination thereof coupled to the housing, and a receptacle coupled to the housing; wherein the capillary blood sample is collected in the receptacle.

Clause 174. The system of clause 173, wherein the receptacle is removably coupled to the housing.

Clause 175. The system of clauses 170-173, wherein the microsampling device further comprises a cap coupled to the receptacle, wherein the cap seals the capillary blood sample within the receptacle.

Clause 176. The system of clauses 170-173, wherein the microsampling device further comprises an actuator movable relative to the housing.

Clause 177. The system of clause 171, wherein the plasma separation device is in fluid communication with the aperture at any point along the reaction vessel.

Clause 178. The system of clause 177, wherein the plasma separation device is placed in fluid communication with the aperture at one end, on a side, or in the middle of the reaction vessel.

Clause 179. The system of clause 177, wherein the plasma separation device is placed in fluid communication with the aperture at an end or side of the reaction vessel at an angle.

Clause 180. The system of clauses 170-179, further comprising a transfer tube.

Clause 181. The system of clause 180, wherein the transfer tube comprises a cap or a stopper.

Clause 182. The system of clauses 171-181, wherein the plasma separation device includes an inlet to receive the capillary blood sample from the microsampling device and an outlet through which the processed capillary blood sample leaves the plasma separation device.

Clause 183. The system of clause 182, wherein the outlet of the plasma separation device is in fluid communication with the aperture of the reaction vessel.

Clause 184. The system of clause 182, wherein the outlet of the plasma separation device is in fluid communication with the cap or stopper of the transfer tube.

Clause 185. The system of clause 184, wherein the cap or stopper of the transfer tube is in fluid communication with the aperture of the reaction vessel.

Clause 186. The system of clauses 173-185, wherein the receptacle is squeezed to force the capillary blood sample through the plasma separation device and into the reaction vessel or transfer tube.

Clause 187. The system of clauses 173-185, wherein the receptacle includes a plunger to force the capillary blood sample through the plasma separation device and into the reaction vessel or transfer tube.

Clause 188. The system of clauses 171-187, wherein the plasma separation device is integrated within the receptacle.

Clause 189. The system of clause 188, wherein the receptacle is a reaction vessel.

Clause 190. The system of clauses 171-189, wherein the plasma separation device is integrated within the reaction vessel.

Clause 191. The system of clauses 171-187, wherein the plasma separation device is integrated into the transfer tube.

Clause 192. The system of clauses 25-45, wherein the plasma separation device includes a filter, a membrane, a synthetic paper, or any combinations thereof.

Clause 193. The system of clauses 170-192, wherein the amount of (i) UCH-L1, GFAP, or UCH-L1 and GFAP; and/or (ii) CK-MB, β-hCG, TSH, homocysteine, free T4 or any combinations thereof, is determined in (a) about 25 minutes from the time the sample is collected; (b) less than about 20 minutes from the time the sample is collected; (c) about 4 to about 20 minutes from the time the sample is collected; (d) about 15 to about 18 minutes from the time the sample is collected; or (e) less than about 18 minutes from the time the sample is collected.

Clause 194. The system of clause 193, wherein the amount of (i) UCH-L1, GFAP, or UCH-L1 and GFAP; or (ii) CK-MB, β-hCG, TSH, homocysteine, free T4 or any combination thereof, is communicated by the instrument.

Clause 195. The system of clause 194, wherein the amount of the (i) UCH-L1, GFAP, or UCH-L1 and GFAP; or (ii) CK-MB, β-hCG, TSH, homocysteine, free T4, TSH, homocysteine, free T4 or any combination thereof, is communicated in a document and/or spreadsheet, on a mobile device, on a computer, on a website, in an e-mail, or any combination thereof.

Clause 196. The system of clause 194 or clause 195, wherein the amount of (i) UCH-L1, GFAP, or UCH-L1 and GFAP; or (ii) CK-MB, β-hCG, TSH, homocysteine, free T4 or any combination thereof is communicated by displaying on the instrument.

Clause 197. The system of clauses 170-196, wherein at least a portion of the system is usable in a decentralized setting.

Clause 198. The method of clause 154, wherein the sample is processed by centrifugation.

Clause 199. The method of clause 150, wherein the subject is diagnosed as having a traumatic brain injury.

Clause 200. The method of clause 199, wherein the subject is treated for the traumatic brain injury.

Clause 201. The method of clause 150, wherein the plasma separation device is an apparatus comprising:
a hydrophobic layer comprising at least one microchannel having a first and second end and which defines a path for capillary fluid flow; and
a top layer flanking the hydrophobic layer, wherein a surface of the top layer facing the hydrophobic layer is hydrophilic, wherein the sample comprises blood or blood products.

Clause 202. The method of clause 201, wherein the apparatus further comprises a bottom layer flanking the hydrophobic layer, wherein a surface of the bottom layer facing the hydrophobic layer is hydrophilic.

Clause 203. The method of clause 201 or clause 202, wherein the top layer, hydrophobic layer, bottom layer, or any combination thereof are adherent to each other.

Clause 204. The method of clauses 201-203, wherein the at least one microchannel extends longitudinally along a portion of the hydrophobic layer to an opening at a second end.

Clause 205. The method of clauses 201-204 wherein the at least one microchannel is less than about 80 mm in length.

Clause 206. The method of clauses 201-205, wherein the at least one microchannel is less than about 5 mm wide.

Clause 207. The method of clauses 201-206, wherein the top layer, bottom layer or top and bottom layers are entirely hydrophilic.

Clause 208. The method of clauses 201-207, wherein a composition of the entirety of the top layer, bottom layer, or both the top and bottom layers each comprise same or different materials.

Clause 209. The method of clauses 201-208, wherein the hydrophobic layer, top layer, bottom layer or any combination thereof have a combined thickness of about 100 to about 600 microns.

Clause 210. The method of clauses 201-209, wherein the hydrophobic layer has thickness of about 50 to about 200 microns.

Clause 211. The method of clauses 201-210, wherein each of the top layer, bottom layer, or top and bottom layers have a thickness of about 50 to about 200 microns.

Clause 212. The method of clauses 201-211, wherein the top layer comprises a sample inlet.

Clause 213. The method of clause 212, wherein the hydrophobic layer and optionally, the bottom layer, comprise an opening below the sample inlet.

Clause 214. The method of clause 212 or clause 213, wherein the sample inlet comprises a separation membrane.

Clause 215. The method of clause 214, wherein the separation membrane is a plasma separation membrane.

Clause 216. The method of clause 214 or clause 215, further comprising a hydrophilic mesh or hydrophilic film positioned above the separation membrane, below the separation membrane, or both above and below the separation membrane.

Clause 217. The method of clauses 213-216, wherein the opening in the hydrophobic layer is connected to the first end of the at least one microchannel.

Clause 218. The method of clauses 201-217, further comprising an agglutinating agent.

Clause 219. The method of clause 218, wherein the agglutinating agent comprises lectin, Merquat-100, Concanavalin A, DEAE-Dextran, poly-L-lysine, polyvinylpyrrolidone, poly(2-(dimethylamino)ethylmethacrylate), or any combinations thereof.

Clause 220. The method of clause 150, wherein the plasma separation device is an apparatus comprising:
a pre-evacuated container or tube having an inlet and outlet end;
a blood holding chamber at the inlet end of the container or tube;
a detachable serum holding chamber at the outlet end of the container or tube;
a filter located within the container between the blood holding chamber and the serum holding chamber.

Clause 221. The method of clause 220, wherein the filter permits the passage of particles or molecules smaller than about 0.7 microns, about 0.6 microns, about 0.5 microns, about 0.4 microns, or about 0.3 microns.

Clause 222. The method of clause 220 or clause 221, wherein a pressure differential between the blood holding chamber and the serum holding chamber allows for whole blood to travel from the blood holding chamber through the filter to produce serum and/or plasma which is collected in the serum holding chamber.

Clause 223. The system of clauses 171-192, wherein the plasma separation device comprises an apparatus having:
a) a hydrophobic layer comprising at least one microchannel having a first and second end and which defines a path for capillary fluid flow; and a top layer flanking the hydrophobic layer, wherein a surface of the top layer facing the hydrophobic layer is hydrophilic; or
b) a pre-evacuated container or tube having an inlet and outlet end; a blood holding chamber at the inlet end of the container or tube; a detachable serum holding chamber at the outlet end of the container or tube; a filter located within the container between the blood holding chamber and the serum holding chamber.

Clause 224. The system of clause 223, wherein the apparatus in a) further comprises a bottom layer flanking the hydrophobic layer, wherein a surface of the bottom layer facing the hydrophobic layer is hydrophilic.

Clause 225. The system of clause 223 or clause 224, wherein the top layer, hydrophobic layer, bottom layer, or any combination thereof are adherent to each other.

Clause 226. The system of clauses 223-225, wherein the at least one microchannel extends longitudinally along a portion of the hydrophobic layer to an opening at a second end.

Clause 227. The system of clauses 223-226 wherein the at least one microchannel is less than about 80 mm in length.

Clause 228. The system of clauses 223-227, wherein the at least one microchannel is less than about 5 mm wide.

Clause 229. The system of clauses 223-227, wherein the top layer, bottom layer or top and bottom layers are entirely hydrophilic.

Clause 230. The system of clauses 223-229, wherein a composition of the entirety of the top layer, bottom layer, or both the top and bottom layers each comprise same or different materials.

Clause 231. The system of clauses 223-230, wherein the hydrophobic layer, top layer, bottom layer or any combination thereof have a combined thickness of about 100 to about 600 microns.

Clause 232. The system of clauses 223-231, wherein the hydrophobic layer has thickness of about 50 to about 200 microns.

Clause 233. The system of clauses 223-232, wherein each of the top layer, bottom layer, or top and bottom layers have a thickness of about 50 to about 200 microns.

Clause 234. The system of clauses 223-233, wherein the top layer comprises a sample inlet.

Clause 235. The system of clause 234, wherein the hydrophobic layer and optionally, the bottom layer, comprise an opening below the sample inlet.

Clause 236. The system of clause 234 or clause 235, wherein the sample inlet comprises a separation membrane.

Clause 237. The system of clause 236, wherein the separation membrane is a plasma separation membrane.

Clause 238. The system of clause 236 or clause 237, further comprising a hydrophilic mesh or hydrophilic film positioned above the separation membrane, below the separation membrane, or both above and below the separation membrane.

Clause 239. The system of clauses 89-92, wherein the opening in the hydrophobic layer is connected to the first end of the at least one microchannel.

Clause 240. The system of clauses 223-239, further comprising an agglutinating agent.

Clause 241. The system of clause 240, wherein the agglutinating agent comprises lectin, Merquat-100, Concanavalin A, DEAE-Dextran, poly-L-lysine, polyvinylpyrrolidone, poly(2-(dimethylamino)ethylmethacrylate), or any combinations thereof.

Clause 242. The system of clause 223, wherein the filter permits the passage of particles or molecules smaller than about 0.7 microns, about 0.6 microns, about 0.5 microns, about 0.4 microns, or about 0.3 microns.

Clause 243. The system of clause 223 or clause 242, wherein a pressure differential between the blood holding chamber and the serum holding chamber allows for whole blood to travel from the blood holding chamber through the filter to produce serum and/or plasma which is collected in the serum holding chamber.

Clause 244. A method comprising:
performing at least one assay for (i) ubiquitin carboxy-terminal hydrolase L1 (UCH-L1), glial fibrillary acidic protein (GFAP), or a combination thereof; and/or (2) CK-MB, β-hCG, thyroid stimulating hormone (TSH), homocysteine, free thyroxine (free T4) or any combination thereof, on a blood sample obtained from a subject to determine an amount of (i) UCH-L1, GFAP, or a combination thereof; and/or (ii) CK-MB, β-hCG, TSH, homocysteine, free T4, or any combination thereof; and
communicating the amount of (i) UCH-L1, GFAP, or combination thereof; and/or (ii) CK-MB, β-hCG, TSH, homocysteine, free T4, or any combination thereof, determined in the sample,
wherein the sample is collected with the use of a syringe, standard needle, or combination thereof; and
further wherein the sample is processed prior to performing the assay with a plasma separation device comprising an apparatus having:
a) a hydrophobic layer comprising at least one microchannel having a first and second end and which defines a path for capillary fluid flow; and a top layer flanking the hydrophobic layer, wherein a surface of the top layer facing the hydrophobic layer is hydrophilic; or
b) a pre-evacuated container or tube having an inlet and outlet end;
a blood holding chamber at the inlet end of the container or tube;
a detachable serum holding chamber at the outlet end of the container or tube;
a filter located within the container between the blood holding chamber and the serum holding chamber.

Clause 245. The method of clause 244, wherein the blood sample is a venous blood sample or a capillary blood sample.

Clause 246. The method of clause 244 or clause 245, wherein the sample is collected in a decentralized or a centralized setting.

Clause 247. The method of clauses 244-246, wherein the apparatus in a) further comprises a bottom layer flanking the hydrophobic layer, wherein a surface of the bottom layer facing the hydrophobic layer is hydrophilic.

Clause 248. The method of clause 244 or clause 247, wherein the top layer, hydrophobic layer, bottom layer, or any combination thereof are adherent to each other.

Clause 249. The method of clauses 244-247, wherein the at least one microchannel extends longitudinally along a portion of the hydrophobic layer to an opening at a second end.

Clause 250. The method of clauses 244-249, wherein the at least one microchannel is less than about 80 mm in length.

Clause 251. The method of clauses 244-250, wherein the at least one microchannel is less than about 5 mm wide.

Clause 252. The method of clauses 244-251, wherein the top layer, bottom layer or top and bottom layers are entirely hydrophilic.

Clause 253. The method of clauses 244-252, wherein a composition of the entirety of the top layer, bottom layer, or both the top and bottom layers each comprise same or different materials.

Clause 254. The method of clauses 244-253, wherein the hydrophobic layer, top layer, bottom layer or any combination thereof have a combined thickness of about 100 to about 600 microns.

Clause 255. The method of clauses 244-254, wherein the hydrophobic layer has thickness of about 50 to about 200 microns.

Clause 256. The method of clauses 244-255, wherein each of the top layer, bottom layer, or top and bottom layers have a thickness of about 50 to about 200 microns.

Clause 257. The method of clauses 244-256, wherein the top layer comprises a sample inlet.

Clause 258. The method of clause 257, wherein the hydrophobic layer and optionally, the bottom layer, comprise an opening below the sample inlet.

Clause 259. The method of clause 257 or clause 258, wherein the sample inlet comprises a separation membrane.

Clause 260. The method of clause 259, wherein the separation membrane is a plasma separation membrane.

Clause 261. The method of clause 259 or clause 260, further comprising a hydrophilic mesh or hydrophilic film positioned above the separation membrane, below the separation membrane, or both above and below the separation membrane.

Clause 262. The method of clauses 256-261, wherein the opening in the hydrophobic layer is connected to the first end of the at least one microchannel.

Clause 263. The method of clauses 244-260, further comprising an agglutinating agent.

Clause 264. The method of clause 263, wherein the agglutinating agent comprises lectin, Merquat-100, Concanavalin A, DEAE-Dextran, poly-L-lysine, polyvinylpyrrolidone, poly(2-(dimethylamino)ethylmethacrylate), or any combinations thereof.

Clause 265. The method of clause 244, wherein the filter permits the passage of particles or molecules smaller than about 0.7 microns, about 0.6 microns, about 0.5 microns, about 0.4 microns, or about 0.3 microns.

Clause 266. The method of clause 244 or clause 265, wherein a pressure differential between the blood holding chamber and the serum holding chamber allows for whole blood to travel from the blood holding chamber through the filter to produce serum and/or plasma which is collected in the serum holding chamber.

Clause 267. A system comprising:
a plasma separation device to process a whole blood sample obtained from a subject into serum and/or plasma;
a reaction vessel that receives the serum and/or plasma from the subject and comprises an assay for (i) ubiquitin carboxy-terminal hydrolase L1 (UCH-L1), glial fibrillary acidic protein (GFAP), or a combination thereof; and/or (ii) CK-MB, β-hCG, thyroid stimulating hormone (TSH), homocysteine, free thyroxine (free T4), or any combination thereof; and
an instrument to analyze the reaction vessel to provide an amount of (i) UCH-L1, GFAP, or UCH-L1 and GFAP; and/or (ii) CK-MB, β-hCG, TSH, homocysteine, free T4, or any combination thereof, in the sample,
wherein the plasma separation device comprises an apparatus having:
a) a hydrophobic layer comprising at least one microchannel having a first and second end and which defines a path for capillary fluid flow; and a top layer flanking the hydrophobic layer, wherein a surface of the top layer facing the hydrophobic layer is hydrophilic; or
b) a pre-evacuated container or tube having an inlet and outlet end;
a blood holding chamber at the inlet end of the container or tube;
a detachable serum holding chamber at the outlet end of the container or tube;
a filter located within the container between the blood holding chamber and the serum holding chamber.

Clause 268. The system of clause 267, wherein the blood sample is a venous blood sample or a capillary blood sample.

Clause 269. The system of clause 267 or clause 268, wherein the sample is collected in a decentralized or a centralized setting.

Clause 270. The system of clause 269, wherein the apparatus in a) further comprises a bottom layer flanking the hydrophobic layer, wherein a surface of the bottom layer facing the hydrophobic layer is hydrophilic.

Clause 271. The system of clause 267 or clause 270, wherein the top layer, hydrophobic layer, bottom layer, or any combination thereof are adherent to each other.

Clause 272. The system of clauses 267-271, wherein the at least one microchannel extends longitudinally along a portion of the hydrophobic layer to an opening at a second end.

Clause 273. The system of clauses 267-272, wherein the at least one microchannel is less than about 80 mm in length.

Clause 274. The system of clauses 267-272, wherein the at least one microchannel is less than about 5 mm wide.

Clause 275. The system of clauses 267-272, wherein the top layer, bottom layer or top and bottom layers are entirely hydrophilic.

Clause 276. The system of clauses 267-275, wherein a composition of the entirety of the top layer, bottom layer, or both the top and bottom layers each comprise same or different materials.

Clause 277. The system of clauses 267-276, wherein the hydrophobic layer, top layer, bottom layer or any combination thereof have a combined thickness of about 100 to about 600 microns.

Clause 278. The system of clauses 267-277, wherein the hydrophobic layer has thickness of about 50 to about 200 microns.

Clause 279. The system of clauses 267-278, wherein each of the top layer, bottom layer, or top and bottom layers have a thickness of about 50 to about 200 microns.

Clause 280. The system of clauses 267-279, wherein the top layer comprises a sample inlet.

Clause 281. The system of clause 280, wherein the hydrophobic layer and optionally, the bottom layer, comprise an opening below the sample inlet.

Clause 282. The system of clause 280 or clause 281, wherein the sample inlet comprises a separation membrane.

Clause 283. The system of clause 282, wherein the separation membrane is a plasma separation membrane.

Clause 284. The system of clause 282 or clause 283, further comprising a hydrophilic mesh or hydrophilic film positioned above the separation membrane, below the separation membrane, or both above and below the separation membrane.

Clause 285. The system of clauses 281-284, wherein the opening in the hydrophobic layer is connected to the first end of the at least one microchannel.

Clause 286. The system of clauses 267-285, further comprising an agglutinating agent.

Clause 287. The system of clause 286, wherein the agglutinating agent comprises lectin, Merquat-100, Concanavalin A, DEAE-Dextran, poly-L-lysine, polyvinylpyrrolidone, poly(2-(dimethylamino)ethylmethacrylate), or any combinations thereof.

Clause 288. The system of clause 267, wherein the filter permits the passage of particles or molecules smaller than about 0.7 microns, about 0.6 microns, about 0.5 microns, about 0.4 microns, or about 0.3 microns.

Clause 289. The system of clause 267 or clause 288, wherein a pressure differential between the blood holding chamber and the serum holding chamber allows for whole blood to travel from the blood holding chamber through the filter to produce serum and/or plasma which is collected in the serum holding chamber.

What is claimed is:

1. A method comprising:
(A) performing at least one assay for (i) ubiquitin carboxy-terminal hydrolase L1 (UCH-L1), glial fibrillary acidic protein (GFAP), or a combination thereof; or (ii) CK-MB, β-hCG, thyroid stimulating hormone (TSH), homocysteine, free thyroxine (free T4), or any combination thereof, on a capillary blood sample obtained from a subject to determine an amount of (i) UCH-L1, GFAP, or a combination thereof; or (ii) CK-MB, β-hCG, TSH, homocysteine, free T4, or any combination thereof; and
(B) communicating the amount of (i) UCH-L1, GFAP, or combination thereof; or (ii) CK-MB, β-hCG, TSH, homocysteine, free T4, or any combination thereof, determined in the sample,
using a point-of-care device or a non-point-of-care device,
wherein the sample is collected from a location on the subject other than a digit and (1) in a decentralized setting; (2) without the use of a syringe, standard needle, or combination thereof; (3) by a user not trained in collecting blood samples from a subject; (4) by a robot; (5) by a self-or other-administered blood collection device; or (6) any combination of (1)-(5), and
further wherein: (i) the assay is capable of being performed in less than about 30 minutes; (ii) the amount of (a) UCH-L1, GFAP, or combination thereof; or (B) CK-MB, β-hCG, TSH, homocysteine, free T4, or any combination thereof, determined in the sample is capable of being communicated in less than about 30 minutes from the time the sample is collected; or (iii) a combination of (i) and (ii).

2. The method of claim 1, wherein the sample is collected using a microsampling device.

3. The method of claim 1, wherein the sample is processed prior to performing the assay and further wherein the sample is whole blood and is processed into serum or plasma.

4. The method of claim 3, wherein the sample is processed using a plasma separation device.

5. The method of claim 2, wherein the microsampling device: (a) comprises a plasma separation device; or (b) is operably linked to the plasma separation device.

6. (canceled)

7. (canceled)

8. (canceled)

9. (canceled)

10. (canceled)

11. (canceled)

12. (canceled)

13. A method comprising:
performing at least one assay for (i) ubiquitin carboxy-terminal hydrolase L1 (UCH-L1), glial fibrillary acidic protein (GFAP), or a combination thereof; or (2) CK-MB, β-hCG, thyroid stimulating hormone (TSH), homocysteine, free thyroxine (free T4) or any combination thereof, on a blood sample obtained from a subject to determine an amount of (i) UCH-L1, GFAP, or a combination thereof; or (ii) CK-MB, β-hCG, TSH, homocysteine, free T4, or any combination thereof; and
communicating the amount of (i) UCH-L1, GFAP, or combination thereof; or (ii) CK-MB, β-hCG, TSH, homocysteine, free T4, or any combination thereof, determined in the sample,
wherein the sample is collected with the use of a syringe, standard needle, or combination thereof; and
further wherein the sample is processed prior to performing the assay with a plasma separation device comprising an apparatus having:
a) a hydrophobic layer comprising at least one microchannel having a first and second end and which defines a path for capillary fluid flow; and a top layer flanking the hydrophobic layer, wherein a surface of the top layer facing the hydrophobic layer is hydrophilic; or
b) a pre-evacuated container or tube having an inlet and outlet end;
a blood holding chamber at the inlet end of the container or tube;
a detachable serum holding chamber at the outlet end of the container or tube;
a filter located within the container between the blood holding chamber and the serum holding chamber.

14. The method of claim 13, wherein the blood sample is a venous blood sample or a capillary blood sample.

15. The method of claim 13, wherein the sample is collected in a decentralized or a centralized setting.

16. (canceled)

17. The method of claim 13, wherein:
(a) the top layer, bottom layer or top and bottom layers are entirely hydrophilic:
(b) a composition of the entirety of the top layer, bottom layer, or both the top and bottom layers each comprise same or different materials:
(c) the hydrophobic layer, top layer, bottom layer or any combination thereof have a combined thickness of about 100 to about 600 microns;
(d) the hydrophobic layer has thickness of about 50 to about 200 microns:
(e) each of the top layer, bottom layer, or top and bottom layers have a thickness of about 50 to about 200 microns:
(f) the top layer comprises a sample inlet:
(g) the hydrophobic layer and optionally, the bottom layer, comprise an opening below the sample inlet:
(h) the sample inlet comprises a separation membrane:
(i) the opening in the hydrophobic layer is connected to the first end of the at least one microchannel:
(j) the plasma separation device further comprises an agglutinating agent:
(k) the filter permits the passage of particles or molecules smaller than about 0.7 microns, about 0.6 microns, about 0.5 microns, about 0.4 microns, or about 0.3 microns:
(l) a pressure differential between the blood holding chamber and the serum holding chamber allows for whole blood to travel from the blood holding chamber through the filter to produce serum and/or plasma which is collected in the serum holding chamber: or
(m) any combinations of (a)-(l).

18. The method of claim 14, wherein the sample is a capillary blood sample.

19. The method of claim 1, wherein the assay comprises contacting the sample with:
(I) (a) an anti-UCH-L1 antibody that binds to UCH-L1 to determine the amount of UCH-L1 in the sample; (b) an anti-GFAP antibody that binds to GFAP to determine the amount of GFAP in the sample; or (c) a combination of (a) and (b); or
(II) (a) an anti-CK-MB antibody that binds to CK-MB to determine the amount of CK-MB in the sample; (b) an anti-β-hCG antibody that binds to β-hCG to determine the amount of β-hCG in the sample; (c) an anti-TSH antibody that binds to TSH to determine the amount of TSH in the sample; (d) an anti-homocysteine antibody that binds to homocysteine to determine the amount of homocysteine in the sample; (e) an anti-free T4 antibody that binds to free T4 to determine the amount of free T4 in the sample; or (f) any combination of (a) to (c).

20. The method of claim 1,
wherein when the assay is for:
(I) (a) GFAP, the GFAP assay comprises a conversion factor for GFAP in a capillary sample compared to GFAP in a venous sample of about 1.0:1.0; (b) UCH-L1, the UCH-L1 assay comprises a conversion factor for UCH-L1 in a capillary sample compared to UCH-L1 in a venous sample of about 2.5:1.0 to about 1.5:1.0; or (c) a combination of (a) and (b); or
(II) (a) CK-MB, the CK-MB assay comprises a conversion factor for CK-MB in a capillary sample compared to CK-MB in a venous sample of about 0.5:1.0 to about 1:0:1.2; (b) β-hCG, the β-hCG assay comprises a conversion factor for β-hCG in a capillary sample compared to β-hCG in a venous sample of about 0.8:1.0 to about 1.0:1.4; (c) TSH, the TSH assay comprises a conversion factor for TSH in a capillary sample compared to TSH in a venous sample of about 0.75:1.0 to about 1.2:1.0; (c) homocysteine, the homocysteine assay comprises a conversion factor for homocysteine in a capillary sample compared to homocysteine in a venous sample of about 1.2:1.0 to about 0.9:1.0; (c) free T4, the free T4 assay comprises a conversion factor for free T4 in a capillary sample compared to free T4 in a venous sample of about 0.8:1.0 to about 1.2:1.0; or (f) any combination of (a) to (c).

21. (canceled)

22. The method of claim 1, wherein the wherein the amount of (a) UCH-L1, GFAP, and UCH-L1 and UCH-L1 and GFAP; or (b) CK-MB, β-hCG, TSH, homocysteine, free T4, or any combination thereof, is communicated in: (a) about 25 minutes from the time the sample is collected; (b) less than about 20 minutes from the time the sample is collected; (c) about 4 to about 20 minutes from the time the sample is collected; (d) about 15 to about 18 minutes from the time the sample is collected; or (e) less than about 18 minutes from the time the sample is collected.

23. The method of claim 1, wherein: (a) the point-of-care device comprises a cartridge; or (b) the non-point-of-care device is a higher throughput assay analyzer.

24. (canceled)

25. (canceled)

26. (canceled)

27. A system comprising:
a microsampling device to collect a capillary blood sample from a subject;
a reaction vessel that receives the capillary blood sample and comprises an assay for (i) ubiquitin carboxy-terminal hydrolase L1 (UCH-L1), glial fibrillary acidic protein (GFAP), or a combination thereof; or (ii) CK-MB, β-hCG, thyroid stimulating hormone (TSH), homocysteine, free thyroxine (free T4), or any combination thereof; and
an instrument to analyze the reaction vessel to provide an amount of (i) UCH-L1, GFAP, or UCH-L1 and GFAP; or (ii) CK-MB, β-hCG, TSH, homocysteine, free T4, or any combination thereof, in the sample.

28. The system of claim 27, wherein the system further comprises a plasma separation device to create a processed capillary blood sample.

29. (canceled)

30. (canceled)

31. (canceled)

32. (canceled)

33. (canceled)

34. (canceled)

35. (canceled)

36. (canceled)

37. (canceled)

38. A system comprising:
a plasma separation device to process a whole blood sample obtained from a subject into serum and/or plasma;
a reaction vessel that receives the serum and/or plasma from the subject and comprises an assay for (i) ubiquitin carboxy-terminal hydrolase L1 (UCH-L1), glial fibrillary acidic protein (GFAP), or a combination thereof: or (ii) CK-MB, β-hCG, thyroid stimulating hormone (TSH), homocysteine, free thyroxine (free T4), or any combination thereof; and
an instrument to analyze the reaction vessel to provide an amount of (i) UCH-L1, GFAP, or UCH-L1 and GFAP; or (ii) CK-MB, β-hCG, TSH, homocysteine, free T4, or any combination thereof, in the sample,
wherein the plasma separation device comprises an apparatus having:
a) a hydrophobic layer comprising at least one microchannel having a first and second end and which defines a path for capillary fluid flow; and a top layer flanking the hydrophobic layer, wherein a surface of the top layer facing the hydrophobic layer is hydrophilic; or
b) a pre-evacuated container or tube having an inlet and outlet end;
a blood holding chamber at the inlet end of the container or tube;
a detachable serum holding chamber at the outlet end of the container or tube;
a filter located within the container between the blood holding chamber and the serum holding chamber.

39. The system of claim 38, wherein the blood sample is a venous blood sample or a capillary blood sample.

40. The system of claim 38, wherein the sample is collected in a decentralized or a centralized setting.

41. (canceled)

42. (canceled)

43. (canceled)

44. (canceled)

45. (canceled)

46. (canceled)

47. (canceled)

48. (canceled)

49. An apparatus comprising:
machine readable instructions; and
processor circuitry to at execute the machine readable instructions to:
at least one of determine, obtain, or interpret results of at least one assay for:
(i) ubiquitin carboxy-terminal hydrolase L1 (UCH-L1), glial fibrillary acidic protein (GFAP), or a combination thereof:
(ii) CK-MB, β-hCG, thyroid stimulating hormone (TSH), homocysteine, free thyroxine (free T4), or any combination thereof, on a blood sample obtained from a subject to determine an amount of (i) UCH-L1, GFAP, or a combination thereof in the blood sample: or
(iii) CK-MB, β-hCG, TSH, homocysteine, free T4, or any combination thereof in the blood sample; and
cause communication, in less than about 30 minutes from the time the sample is collected, of the amount of (i) UCH-L1, GFAP, or combination thereof; or (ii) CK-MB, BB-hCG, TSH, homocysteine, free T4, or any combination thereof, determined in the sample, wherein the sample is to be collected:
(1) from a location on the subject other than a digit;
(2) in a decentralized setting;
(3) without the use of a syringe, standard needle, or combination thereof;
(4) by a user not trained in collecting blood samples from a subject;
(5) by a robot;
(6) by a self-administered blood collection device, or
(7) any combination of (1)-(6).

50. The apparatus of claim 49, wherein the processor circuitry is to enable performance of the at least one assay.

51. (canceled)

52. The apparatus of claim 49, wherein when the assay is for:
(a) GFAP, the processor circuitry is to apply a conversion factor for GFAP in a capillary sample compared to GFAP in a venous sample of about 1.0:1.0;
(b) UCH-L1, the processor circuitry is to apply a conversion factor for UCH-L1 in a capillary sample compared to UCH-L1 in a venous sample of about 2.5:1.0 to about 1.5:1.0;
(c) CK-MB, the processor circuitry is to apply a conversion factor for CK-MB in a capillary sample compared to CK-MI in a venous sample of about 0.5:1.0 to about 1:0:1.2;
(d) BB-hCG, the processor circuitry is to apply a conversion factor for β-hCG in a capillary sample compared to β-hCG in a venous sample of about 0.8:1.0 to about 1.0:1.4;
(e) TSH, the processor circuitry is to apply a conversion factor for TSH in a capillary sample compared to TSH in a venous sample of about 0.75:1.0 to about 1.2:1.0;
(f) homocysteine, the processor circuitry is to apply a conversion factor for homocysteine in a capillary sample compared to homocysteine in a venous sample of about 1.2:1.0 to about 0.9:1.0; or
(g) free T4, the processor circuitry is to apply a conversion factor for free T4 in a capillary sample compared to free T4 in a venous sample of about 0.8:1.0 to about 1.2:1.0.

53. The apparatus of claim 49, wherein the processor circuitry is to apply a conversion factor for:
(a) GFAP in a capillary sample compared to GFAP in a venous sample of about 1.0:1.0, when the assay is for GFAP:
(b) UCH-L1 in a capillary sample compared to UCH-L1 in a venous sample of about 2.5:1.0 to about 1.5:1.0, when the assay is for UCH-L1:
(c) CK-MB in a capillary sample compared to CK-MB in a venous sample of about 0.5:1.0 to about 1:0:1.2, when the assay is for CK-MO:
(d) β-hCG in a capillary sample compared to β-hCG in a venous sample of about 0.8:1.0 to about 1.0:1.4, when the assay is for β-hCG;
(e) TSH in a capillary sample compared to TSH in a venous sample of about 0.75:1.0 to about 1.2:1.0, when the assay is for TSH;
(f) homocysteine in a capillary sample compared to homocysteine in a venous sample of about 1.2:1.0 to about 0.9:1.0, when the assay is for homocysteine; or
(g) free T4 in a capillary sample compared to free T4 in a venous sample of about 0.8:1.0 to about 1.2:1.0, when the assay is for free T4.

54. (canceled)

55. (canceled)

56. (canceled)

57. (canceled)

58. The apparatus of claim 49, wherein the processor circuitry is to cause communication of the amount of (a) UCH-L1, GFAP, and UCH-L1 and UCH-L1 and GFAP; or (b) CK-MB, β-hCG, TSH, homocysteine, free T4, or any combination thereof, in about 4 to about 20 minutes from the time the sample is collected.

59. (canceled)

60. (canceled)

61. (canceled)

62. A non-transitory machine readable storage medium comprising instructions to cause one or more processors to at least:
access results of at least one assay for:
(i) ubiquitin carboxy-terminal hydrolase L1 (UCH-L1), glial fibrillary acidic protein (GFAP), or a combination thereof;
(ii) CK-MB, β-hCG, thyroid stimulating hormone (TSH), homocysteine, free thyroxine (free T4), or any combination thereof, on a blood sample obtained from a subject to determine an amount of (i) UCH-L1, GFAP, or a combination thereof in the blood sample; or
(iii) CK-MB, β-hCG, TSH, homocysteine, free T4, or any combination thereof in the blood sample; and
cause communication, in less than about 30 minutes from the time the sample is collected, of the amount of (i) UCH-L1, GFAP, or combination thereof; or (ii) CK-MB, β-hCG, TSH, homocysteine, free T4, or any combination thereof, determined in the sample,
wherein the sample is to be collected:
(1) from a location on the subject other than a digit;
(2) in a decentralized setting:
(3) without the use of a syringe, standard needle, or combination thereof;
(4) by a user not trained in collecting blood samples from a subject;
(5) by a robot;
(6) by a self-administered blood collection device, or
(7) any combination of (1)-(6).

63. The storage medium of claim 62, wherein the instructions cause the one or more processors to enable performance of the at least one assay.

64. (canceled)

65. The storage medium of claim 62, wherein the instructions cause the one or more processors to apply a conversion factor for:
(a) GFAP in a capillary sample compared to GFAP in a venous sample of about 1.0:1.0, when the assay is for GFAP;
(b) UCH-L1 in a capillary sample compared to UCH-L1 in a venous sample of about 2.5:1.0 to about 1.5:1.0, when the assay is for UCH-L1;
(c) CK-MB in a capillary sample compared to CK-MB in a venous sample of about 0.5:1.0 to about 1:0:1.2, when the assay is for CK-MO;
(d) β-hCG in a capillary sample compared to β-hCG in a venous sample of about 0.8:1.0 to about 1.0:1.4, when the assay is for β-hCG;
(e) TSH in a capillary sample compared to TSH in a venous sample of about 0.75:1.0 to about 1.2:1.0, when the assay is for TSH;
(f) homocysteine in a capillary sample compared to homocysteine in a venous sample of about 1.2:1.0 to about 0.9:1.0, when the assay is for homocysteine; or
(g) free T4 in a capillary sample compared to free T4 in a venous sample of about 0.8:1.0 to about 1.2:1.0, when the assay is for free T4.

66. (canceled)

67. The storage medium of claim 62, wherein the instructions cause the one or more processors to cause communication of the amount of (a) UCH-L1, GFAP, and UCH-L1 and UCH-L1 and GFAP; or (b) CK-MB, β-hCG, TSH, homocysteine, free T4, or any combination thereof, in about 25 in less than about 18 minutes from the time the sample is collected.

68. (canceled)


SEQUENCE LISTING (5 sequences)

SEQ ID NO: 1 - protein, 223 aa, synthetic construct [sequence omitted]

SEQ ID NO: 2 - protein, 432 aa, synthetic construct
  REGION 1..432: note = Synthetic
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 3 - protein, 6 aa, synthetic construct
  REGION 1..6: note = Synthetic
HHHHHH

SEQ ID NO: 4 - protein, 5 aa, synthetic construct
  REGION 1..5: note = Synthetic
DDDDK

SEQ ID NO: 5 - protein, 6 aa, synthetic construct
  REGION 1..6: note = Synthetic
ADDDDK